(19) United States
(12) Patent Application Publication
(10) Pub. No.: US 2026/0132136 A1
(43) Pub. Date: May 14, 2026

(54) Pyrazolo[5,1-f][1,2,4]triazin-4-ones as inhibitors of NOX4

(71) Applicant: Boehringer Ingelheim International GmbH, Ingelheim am Rhein (DE)

(72) Inventors: Christian GNAMM, Biberach an der Riss (DE); Elmar BAUSCHATZ, Schemmerhofen (DE); Frank H. BUETTNER, Attenweiler (DE); Cédrickx GODBOUT, Schemmerhofen (DE); Annekatrin Charlotte HEIMANN, Biberach an der Riss (DE); Christoph HOENKE, Biberach an der Riss (DE); Ferenc KONTES, Biberach (DE); Christian Andreas KUTTRUFF, Donaueschingen (DE); Dieter WIEDENMAYER, Biberach an der Riss (DE); Raphael WILDERMUTH, Irsee (DE)

(21) Appl. No.: 19/308,750

(22) Filed: Aug. 25, 2025

(30) Foreign Application Priority Data
Aug. 26, 2024 (EP) 24196344.6

(51) Int. Cl. C07D 487/04; A61K 31/4985; A61K 31/53; A61K 45/06

(52) U.S. Cl. CPC C07D 487/04; A61K 31/4985; A61K 31/53; A61K 45/06

(57) ABSTRACT

The present disclosure provides pyrazolo[5,1-f][1,2,4]triazin-4-ones that are inhibitors of NOX4, and are therefore useful for the treatment of diseases treatable by inhibition of NOX4. Also provided are pharmaceutical compositions containing the same, and processes for preparing said compounds.

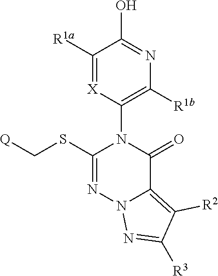

### TECHNICAL FIELD

The present disclosure provides certain pyrazolo-triazinone derivatives that are inhibitors of NOX4, and are therefore useful for the treatment of diseases treatable by inhibition of NOX4. Furthermore, the invention relates to pharmaceutical compositions and combinations comprising these compounds, as well as their use in methods for the treatment of diseases associated with or modulated by NOX4. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

### BACKGROUND INFORMATION

Redox signaling is a critical part of a variety of cell signaling pathways involved in the regulation of cell growth, differentiation, metabolism, immune regulation, and other physiological functions. It is characterized by an oxidation-reduction reaction or covalent adduct formation between the sensor and the second messenger. Thereby oxidation of the sensor protein can modulate its activity, conformation, or susceptibility towards degradation. In case of redox signaling the second messengers consist of a group of reactive oxygen and nitrogen species (ROS, RNS). These are generated as side products during metabolic reactions in the mitochondria but also by specialized enzymes like NADPH-oxidases (NOXes). In parallel, a complex antioxidant system evolved to protect the cells from injury by ROS and RNS, ensuring a tight balance between ROS/RNS formation and degradation. In case this balance is disturbed, oxidative stress can occur leading to increased protein oxidation and aberrant redox signaling. Therefore, oxidative stress is associated with various pathophysiological conditions like interstitial lung diseases, cancer and inflammatory diseases.

Several studies showed over the last decade that in interstitial lung diseases, like idiopathic pulmonary fibrosis (IPF), the redox balance is disturbed¹ and that NOX4 is specifically upregulated in the lungs of IPF patients¹. IPF patients experience a decline in their lung function and blood oxygenation, which is caused by the remodeling of the lung parenchyma in combination with the stiffening of the lung tissue due to increased matrix deposition. Upon epithelial injury due to e.g. infection, air pollutants etc., fibroblasts are activated to enable migration of immune cells as well as epithelial precursor cells to the site of injury. Once the injury is resolved fibroblasts as well as immune cells undergo apoptosis to allow complete restoration of the tissue. In IPF an aberrant repair is occurring leading to a progressive remodeling of the lung parenchyma². Recent studies using single cell sequencing allowed the identification and comparison of different cell types of lung tissue derived from IPF patients or healthy controls. These studies showed increased numbers of fibroblasts as well as a strong dedifferentiation of the epithelial cell population³.

NOX4 is part of the NADPH oxidase enzyme family. NOXes are membrane bound multisubunit protein complexes, which transfer electrons across the plasma membrane to generate ROS. While the activity of all other family members (NOX1, NOX2, NOX3 and NOX5) is tightly controlled and inducible, NOX4 is the only family member that is constitutively active¹. It is localized in the plasma membrane, perinuclear vesicles, the endoplasmatic reticulum (ER), the mitochondria and the nuclear membrane. The generation of hydrogen peroxide (H₂O₂) by NOX4 leads to reversible oxidation of cysteine thiol groups and/or S-glutathionylation¹. Thereby, NOX4 is modulating TGFß signaling and other key pathways known to be involved in disease progression¹.

IPF fibroblasts show higher expression of NOX4 in comparison to fibroblasts isolated from healthy tissue, as well as hyperplastic alveolar cells. The role of NOX4 in fibroblast activation as well as proliferation was investigated in lung fibroblasts¹. Fibroblast proliferation, as well as fibroblast activation are known processes driving disease progression. Immunohistochemistry on control and IPF lung samples confirmed increased NOX4 staining in fibroblasts and showed stronger staining in bronchial and alveolar epithelial cells¹.

Acute respiratory distress syndrome (ARDS) and acute lung injury (ALI) are characterized by pulmonary infiltration and edema. Literature shows that NOX4 is increased in patients and plays a crucial role in cell recruitment in animal models⁴.

Metabolic Dysfunction-associated Steatohepatitis (MASH) is characterized by an increase in lipid content and inflammation in the liver. The chronic feedback loop of lipotoxicity, increased inflammation and cell death leads to liver fibrosis. Throughout that process liver cells, namely stellate cells, dedifferentiate to an activated phenotype, of which one hallmark is the induction of pro-fibrotic and pro-inflammatory expression pattern. As on main redox-mediating signalling pathway, the NOX-family—especially NOX4—plays a crucial role in that feedback loop. NOX4-dependent signalling directly leads to an induction of NFkB and MAPk transcription leading to induction of gene products, such as smooth muscle actin (SMA), collagen or tumor necrosis factor (TNF).

Published literature shows that NOX4 is expressed in stellate cells and is expressed through the process of stellate cell activation. Vice versa, the inhibition of NOX4 on the other hand leads to attenuated expression of stellate cell-activation markers.

In addition to in vitro analyses, studies in NOX4 knock-out mice show reduced fibrosis and inflammation in MASH-relevant models.

Aside from the prevention of fibrosis onset and progression, NOX4 plays a crucial role of endothelial integrity. Thereby, NOX4 activity fosters the nitric oxide (NO) production, leading to vascular relaxation and reduced endothelial inflammation. Following, NOX4 inhibition has a beneficial impact on portal hypertension.

In cancer, ROS deregulation contributes to tumor development, progression, and metastasis. In some instances, this enhanced ROS, driven by various oncogenic perturbations, is required for tumorigenicity leading to the acquisition of further DNA damage and genome instability in cancer cells⁵. Increased ROS occurs through a variety of mechanisms such as increased expression of the NOX proteins or NOX activators, or the downregulation of ROS-regulating systems. NOX4 itself has been reported to be increased in many types of tumors leading to increased proliferation, migration, and apoptosis⁵. Tumor associated macrophages are influenced by ROS and can adopt an immunosuppressive phenotype within tumors in response to ROS⁵. Furthermore, when fibroblasts are educated to become cancer associated fibroblasts (CAFs), NOX4 is upregulated and leads to CD8+ T cell exclusion and immune suppression in tumors. Deletion or inhibition of NOX4 in several mouse models of cancer, has reversed this immune suppression and restored immunotherapy response to anti-PD-1 therapy (PD1 being programmed cell death protein 1)⁵. Therefore, specific inhibition of NOX proteins in tumor cells themselves or stromal cells, is a tractable target for anti-tumor therapies.

The list of the references cited above can be found here:
1. Veith, C., Boots, A. W., Idris, M., Schooten, F.-J. van & Vliet, A. van der. Redox Imbalance in Idiopathic Pulmonary Fibrosis: A Role for Oxidant Cross-Talk Between NADPH Oxidase Enzymes and Mitochondria. Antioxid Redox Sign 31, 1092-1115 (2019).
2. Hinz, B. & Lagares, D. Evasion of apoptosis by myofibroblasts: a hallmark of fibrotic diseases. Nat Rev Rheumatol 16, 11-31 (2020).
3. Habermann, A. C. et al. Single-cell RNA sequencing reveals profibrotic roles of distinct epithelial and mesenchymal lineages in pulmonary fibrosis. Sci Adv 6, eaba1972 (2020).
4. Lee, S. H. et al. NADPH oxidase 4 signaling in a ventilator-induced lung injury mouse model. Respir Res 23, 73 (2022).
5. Cheung, E. C. & Vousden, K. H. The role of ROS in tumour development and progression. Nat Rev Cancer 22, 280-297 (2022).

### DETAILED DESCRIPTION OF THE INVENTION

The present invention discloses novel pyrazolo-triazinone derivatives that are inhibitors of NOX4, possessing appropriate pharmacological and pharmacokinetic properties enabling their use as medicaments for the treatment of conditions and/or diseases treatable by inhibition of NOX4.

PCT/EP2024/054867 discloses thiazolo-pyrimidinone derivatives as NOX4-inhibitors.

WO2016207785 discloses benzoxazole and benzthiazole based NOX4 inhibitors with the following generalized structural formula

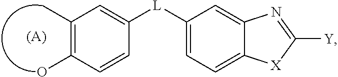

of which example 1, example 15 and example 80 are reported with the following IC₅₀ values in an assay on modified human embryonic kidney (HEK) cells using Amplex Red as detecting reagent:

| Example as dis- | | NOX4 IC₅₀ [μM] as |
|---|---|---|
| closed in | | disclosed in |
| WO2016207785 | Structure | WO2016207785 |
| 1 | 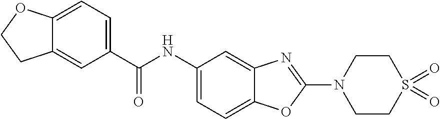 [SMILES: O=C(Nc1ccc2oc(N3CCS(=O)(=O)CC3)nc2c1)c1ccc2c(c1)CCO2] | 0.34 |
| 15 | 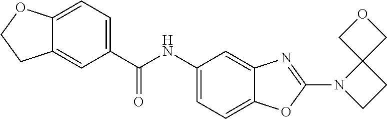 [SMILES: O=C(Nc1ccc2oc(N3CCC34COC4)nc2c1)c1ccc2c(c1)CCO2] | 0.40 |
| 80 | 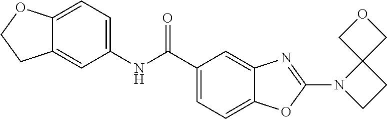 [SMILES: O=C(Nc1ccc2c(c1)CCO2)c1ccc2oc(N3CCC34COC4)nc2c1] | 0.31 |

When tested in an assay for metabolic stability as described herein below, examples 1, 15 and 80 show a clearance in human hepatocytes of 52% QH, 29% QH and 46% QH, respectively.

Another patent application, WO2005049613 discloses bicyclic pyrimidin-4-(3H)-one based modulators of the vanilloid-1 receptor (VR1) of the following generalized structural formula

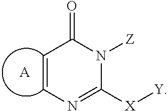

Examples 19, 20, 30, 59 and 63 from WO2005049613 have been tested for their activity on NOX4 inhibition with the assay described herein below, resulting in the following IC₅₀ values:

| Example as | | NOX4 |
|---|---|---|
| disclosed in | | IC₅₀ |
| WO2005049613 | Structure | [μM] |
| 19 | 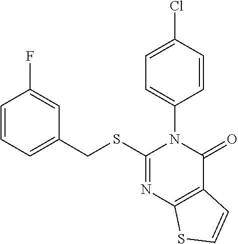 [SMILES: O=c1c2ccsc2nc(SCc2cccc(F)c2)n1-c1ccc(Cl)cc1] | >99 |
| 20 | 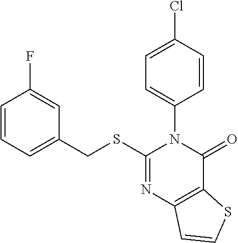 [SMILES: O=c1c2sccc2nc(SCc2cccc(F)c2)n1-c1ccc(Cl)cc1] | >99 |
| 30 | 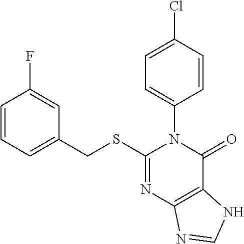 [SMILES: O=c1c2[nH]cnc2nc(SCc2cccc(F)c2)n1-c1ccc(Cl)cc1] | >99 |
| 59 | 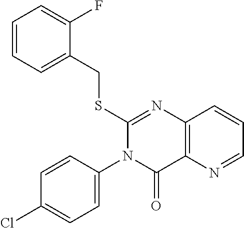 [SMILES: O=c1c2ncccc2nc(SCc2ccccc2F)n1-c1ccc(Cl)cc1] | 79 |
| 63 | 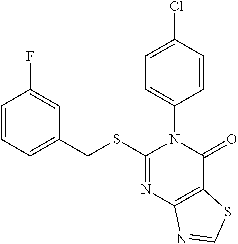 [SMILES: O=c1c2scnc2nc(SCc2cccc(F)c2)n1-c1ccc(Cl)cc1] | 55 |

As visible from the above data, the tested compounds of WO2005049613 show low activity as inhibitors of NOX4.

Surprisingly it was found that the exchange of the para-chloro substituent in example 63 from WO2005049613 with a para-hydroxy substituent increases the inhibition of NOX4 from 55 μM to 0.66 μM with respect to IC₅₀ as tested in the NOX4-inhibition assay described herein below, as shown by the following reference compounds in the following table:

| | | NOX4 |
|---|---|---|
| Example | Structure | IC₅₀ [μM] |
| Reference | 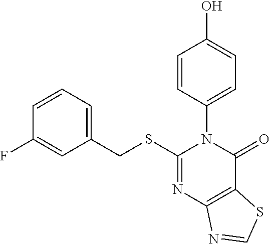 [SMILES: O=c1c2scnc2nc(SCc2cccc(F)c2)n1-c1ccc(O)cc1] | 0.56 |
| Reference | 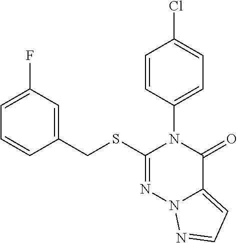 [SMILES: O=c1c2ccnn2nc(SCc2cccc(F)c2)n1-c1ccc(Cl)cc1] | >10 |
| Reference | 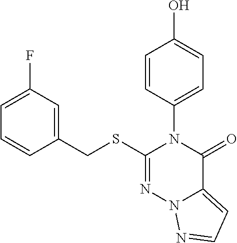 [SMILES: O=c1c2ccnn2nc(SCc2cccc(F)c2)n1-c1ccc(O)cc1] | 1.26 |

The effect of the hydroxy group in this position on the NOX4 inhibitory activity of compounds of the present invention is also shown in the following table:

| | | NOX4 |
|---|---|---|
| Example | Structure | IC₅₀ [μM] |
| Reference Example | 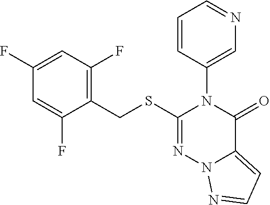 [SMILES: O=c1c2ccnn2nc(SCc2c(F)cc(F)cc2F)n1-c1cccnc1] | 6.32 |
| Reference Example | 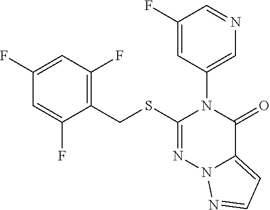 [SMILES: O=c1c2ccnn2nc(SCc2c(F)cc(F)cc2F)n1-c1cncc(F)c1] | 4.75 |
| Example 167 of the present invention | 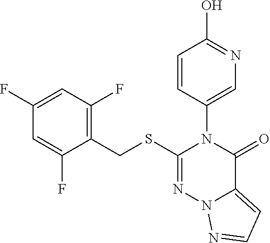 [SMILES: O=c1c2ccnn2nc(SCc2c(F)cc(F)cc2F)n1-c1ccc(O)nc1] | 0.28 |
| Example 127 of the present invention | 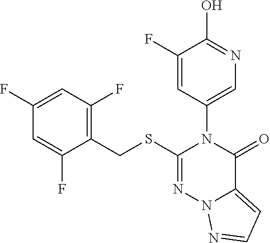 [SMILES: O=c1c2ccnn2nc(SCc2c(F)cc(F)cc2F)n1-c1cnc(O)c(F)c1] | 0.11 |
| Reference | 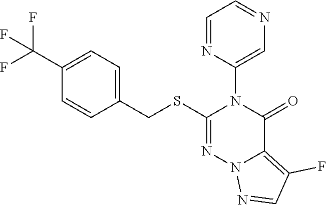 [SMILES: O=c1c2c(F)cnn2nc(SCc2ccc(C(F)(F)F)cc2)n1-c1cnccn1] | 2.66 |
| Example 47 of the present invention | 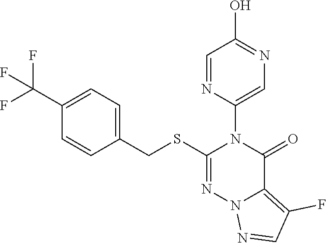 [SMILES: O=c1c2c(F)cnn2nc(SCc2ccc(C(F)(F)F)cc2)n1-c1cnc(O)cn1] | 0.020 |
| Example 25 of the present invention | 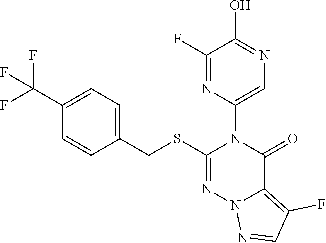 [SMILES: O=c1c2c(F)cnn2nc(SCc2ccc(C(F)(F)F)cc2)n1-c1cnc(O)c(F)n1] | 0.073 |
| Reference | 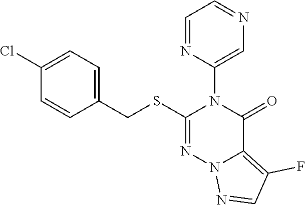 [SMILES: O=c1c2c(F)cnn2nc(SCc2ccc(Cl)cc2)n1-c1cnccn1] | 1.67 |
| Example 45 of the present invention | 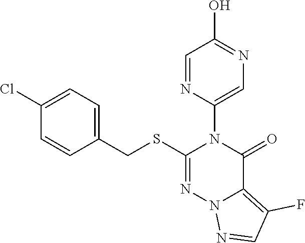 [SMILES: O=c1c2c(F)cnn2nc(SCc2ccc(Cl)cc2)n1-c1cnc(O)cn1] | 0.014 |

It is therefore the aim of the present invention to provide pyrazolo-triazinones which are potent, metabolically stable and selective NOX4 inhibitors.

The compounds of the present invention according to general formula (I)

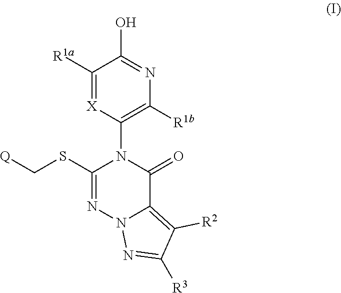

wherein
Q is

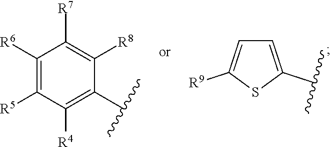

X is N or C—R^{1c}; R^{1a}, R^{1b} and R^{1c} is H or F; R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; R³ is selected from among a group consisting of —H, —C₁₋₄-alkyl, —CN, and halogen; R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; —R⁹ is halogen or —C₁₋₄-haloalkyl; or a salt thereof; exhibit several advantageous properties such as high potency, high metabolic stability and high selectivity for NOX4 and against NOX1, NOX2, NOX3 and NOX5.

The compounds according to the invention typically show inhibition of NOX4 with IC₅₀-values below 600 nM, preferably below 400 nM, more preferably below 200 nM, most preferably below 100 nM (see assay description herein below and table 1). High potency can enable lower doses for pharmacological efficacy. Lower doses have the advantages of lower “drug load” or “drug burden” (parent drug and metabolites thereof) for the patient causing potentially less side effects, and lower production costs for the drug product.

In a further aspect of the invention, the compounds according to the invention are selective NOX4-inhibitors, and are selective against NOX1, NOX2, NOX3 and NOX5. Preferred are compounds in which the IC₅₀ on NOX4 is 10× lower than the IC₅₀ on either of NOX1, NOX2, NOX3 or NOX5. More preferred are compounds in which the IC₅₀ on NOX4 is 30× lower than the IC₅₀ on either of NOX1, NOX2, NOX3 or NOX5. Most preferred are compounds in which the IC₅₀ on NOX4 is 100× lower than the IC₅₀ on either of NOX1, NOX2, NOX3 or NOX5 (see assay description herein below and table 2).

Furthermore, the compounds according to the invention are metabolically stable as shown in human hepatocytes. Metabolic stability in human hepatocytes in this respect is defined as below or equal to 45% QH, preferably below or equal to 30% QH, more preferably below or equal to 20% QH (see assay description herein below and table 3 and the definition of how to calculate the % QH=hepatic blood flow herein below). Therefore, the compounds of the present invention are expected to have a favorable in vivo clearance and thus the favourably long duration of action in humans. Stability in human hepatocytes refers to the susceptibility of compounds to biotransformation in the context of selecting and/or designing drugs with favorable pharmacokinetic properties, as the primary site of metabolism for many drugs is the liver. Human hepatocytes contain the cytochrome P450 (CYPs) and additional enzymes for phase II metabolism (e.g. phosphatases and sulfatases), and thus represent a model system for studying in vitro how a drug is metabolised. Stability in hepatocytes is associated with several advantages, including improved bioavailability and half-life, which can allow lower and less frequent dosing in patients. Thus, stability in hepatocytes is a favorable characteristic for compounds that are to be used as drugs in the treatment of a disease.

### USED TERMS AND DEFINITIONS

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached. In case a chemical bond at the end of moiety is indicated by using a “-”, this “-” signals the attachment point of the group to the molecule.

In case a compound of the present invention is depicted in the form of a chemical name and as a formula, in case of any discrepancy the formula shall prevail. A wavy line may be used in sub-formulas to indicate the bond which is connected to the core molecule as defined.

For example, the term “3-carboxypropyl-group” represents the following substituent:

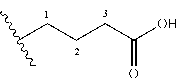 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

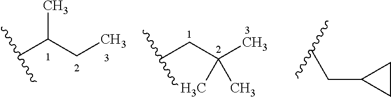

The wavy line may be used in sub-formulas to indicate the bond which is connected to the core molecule as defined.

### Term Substituted

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like. Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

### Salts

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof. Salts of other acids than those mentioned above which for example are useful for purifying or isolating the compounds of the present invention (e.g. trifluoro acetate salts,) also comprise a part of the invention.

### Halogen

The term halogen denotes fluorine, chlorine, bromine and iodine.

### Alkyl

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

### Alkylene

The term “C₁₋ₙ-alkylene” wherein n is an integer selected from 2, 3, 4, 5 or 6, preferably 4, 5 or 6, either alone or in combination with another radical, denotes an acyclic, saturated, branched or linear chain divalent alkyl radical containing from 1 to n carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(CH₂CH₃)—, —CH(CH₂CH₃)—CH₂—, —CH(CH₂CH₂CH₃)—, —CH(CH(CH₃))₂— and —C(CH₃)(CH₂CH₃)—.

### Alkenyl

The term “C₂₋ₘ-alkenyl” is used for a group “C₂₋ₘ-alkyl” wherein m is an integer selected from 3, 4, 5 or 6, preferably 4, 5 or 6, if at least two carbon atoms of said group are bonded to each other by a double bond.

### Alkenylene

The term “C₂₋ₘ-alkenylene” is used for a group “C₂₋ₘ-alkylene”, wherein m is an integer selected from 3, 4, 5 or 6, preferably 4, 5 or 6, if at least two carbon atoms of said group are bonded to each other by a double bond.

### Alkynyl

The term “C₂₋ₘ-alkynyl” is used for a group “C₂₋ₘ-alkyl” wherein m is an integer selected from 3, 4, 5 or 6, preferably 4, 5 or 6, if at least two carbon atoms of said group are bonded to each other by a triple bond.

### Alkynylene

The term “C₂₋ₘ-alkynylene” is used for a group “C₂₋ₘ-alkylene” wherein m is an integer selected from 3, 4, 5 or 6, preferably 4, 5 or 6, if at least two of those carbon atoms of said group are bonded to each other by a triple bond.

### Cycloalkyl

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

### Halo-(Alkyl, Alkylene or Cycloalkyl)

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: F₃C—, H₂FC—, HF₂C.

### Heteroaryl

The term “heteroaryl” means a mono- or polycyclic ring system, comprising at least one aromatic ring, containing one or more heteroatoms selected from N, O, S, SO or SO₂, consisting of 5 to 14 ring atoms wherein at least one of the heteroatoms is part of an aromatic ring. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

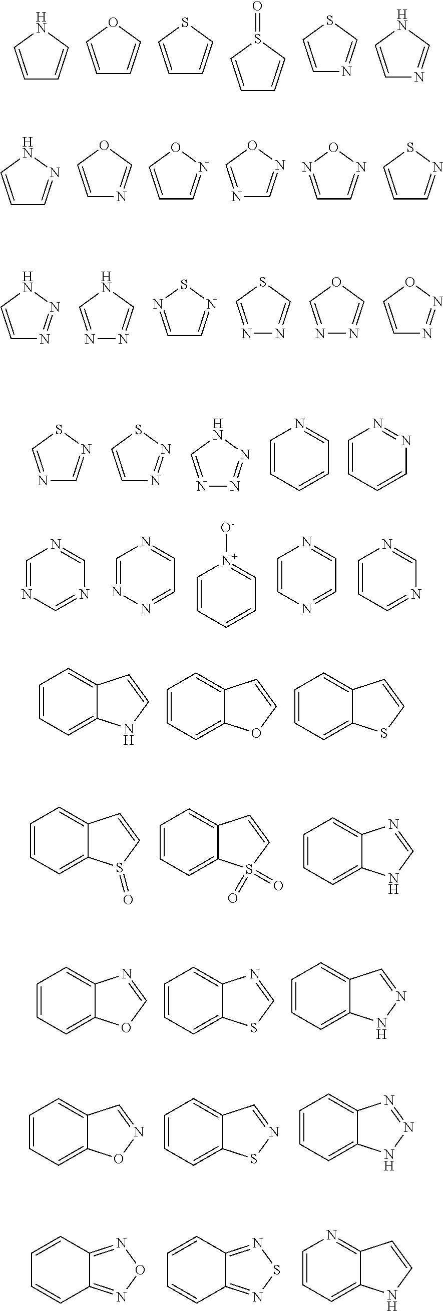

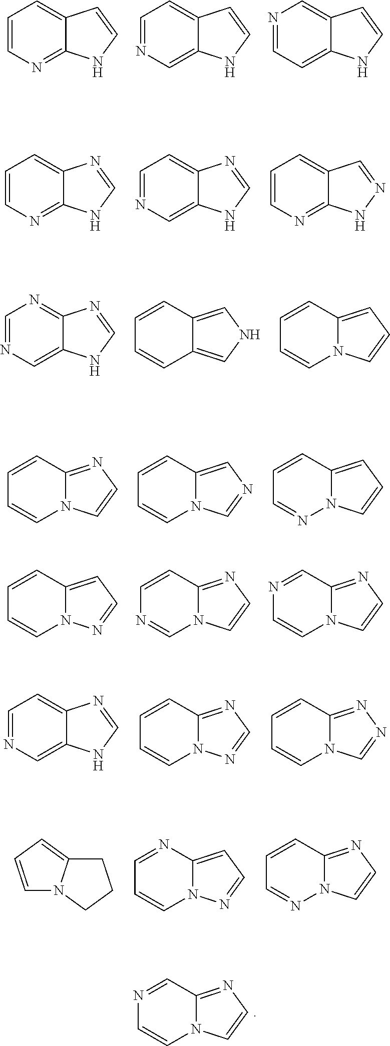

Many of the terms given above may be used repeatedly in the definition of a formula or group and in each case have one of the meanings given above, independently of one another.

The term “bicyclic ring systems” means groups consisting of 2 joined cyclic substructures including spirocyclic, fused, and bridged ring systems.

### PREFERRED EMBODIMENTS

In another embodiment the invention relates to compounds of general formula (Ia)

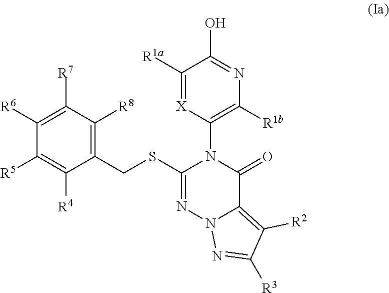

wherein X is N or C—R^{1c}; and wherein R^{1c} is —H; and wherein R^{1a} and R^{1b} are —H or —F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C2-4-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is —H; and wherein R^{1a} and R^{1b} are —H or —F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ia)
wherein X is N or C—R^{1c}; and wherein R^{1c} is —H; and wherein R^{1a} and R^{1b} are H or F but provided that not both R^{1a} and R^{1b} are F simultaneously; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least 2 R⁴, R⁵, R⁶, R⁷, R⁸ are hydrogen; or a salt thereof.

In another embodiment the invention relates to compounds of general formula (Ia)
wherein X is N or C—R^{1c}; and wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ia)
wherein X is N or C—R^{1c}; and wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Ib)

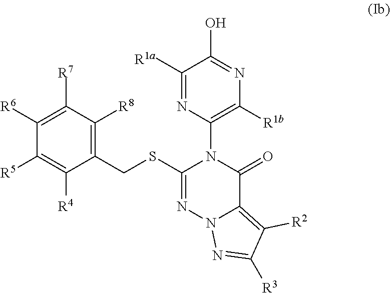

wherein R^{1a} and R^{1b} are H or F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H or F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1c} is H; and wherein R^{1a} and R^{1b} are H or F but provided that not both R^{1a} and R^{1b} are F simultaneously; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R5 is H; and wherein R7 is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R5 is H; and wherein R7 is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Ic)

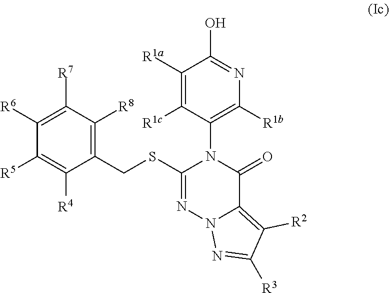

wherein R^{1c} is H; and wherein R^{1a} and R^{1b} are H or F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} are H or F; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1c} is H; and wherein R^{1a} and R^{1b} are H or F but provided that not both R^{1a} and R^{1b} are F simultaneously; and wherein R² is selected from among a group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic) wherein R^{1c} is H; and wherein R^{1a} and R^{1b} are H or F but provided that not both R^{1a} and R^{1b} are F simultaneously; and wherein R² is selected from among a group consisting of —H, —F, —Cl, Br, I, —CH₃, —CH₂CH₃, -cyclopropyl, —CF₃, —CF₂H, —CH₂F, —CN, —CH═CH₂, —CH═CHF, —CH═CF₂, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is selected from among a group consisting of —H, —C₁₋₄-alkyl, —CN, and halogen; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Id)

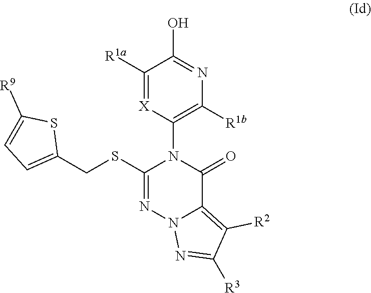

wherein X is N or C—R^{1c}; and wherein R^{1c} is H; and wherein R⁹ is —Cl or —CF₃; or a salt thereof.

In another embodiment the invention relates to compounds of general formula (Id)
wherein X is N or C—R^{1c}; and wherein R^{1c} is H; and wherein R² and R³ is H; and wherein R⁹ is —Cl or —CF₃; or a salt thereof.

In another embodiment the invention relates to compounds of general formula (Ie)

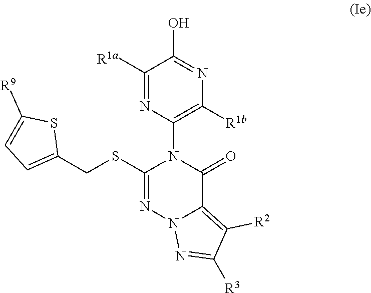

wherein R² and R³ is H; and wherein R⁹ is —Cl or —CF₃; or a salt thereof.

In another embodiment the invention relates to compounds of general formula (Ia)

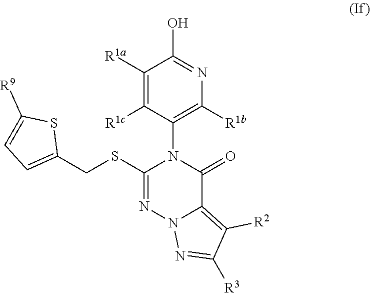

wherein R^{1c} is H; and wherein R² and R³ is H; and wherein R⁹ is —Cl or —CF₃; or a salt thereof.

In another embodiment the invention relates to compounds of general formula (Ia)
wherein X is N or CR^{1c}; and wherein R^{1a}, R^{1b} and R^{1c} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or CR^{1c}; and wherein R^{1a}, R^{1b} and R^{1c} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ia)
wherein X is N or CR^{1c}; and wherein R^{1a}, R^{1b} and R^{1c} is H or F and wherein R^{1a} and R^{1b} are H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ia)
wherein X is N or CR^{1c}; and wherein R^{1a}, R^{1b} and R^{1c} is H or F and wherein R^{1a} and R^{1b} are H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or CR^{1c}; and wherein R^{1a}, R^{1b} and R^{1c} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Ib)

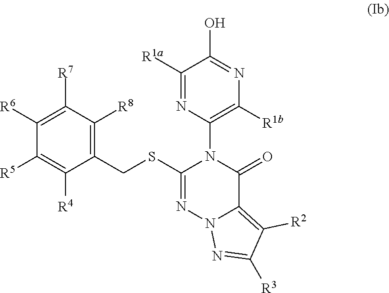

wherein R^{1a} and R^{1b} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ib)
wherein R^{1a} and R^{1b} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1a} and R^{1b} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂C, —OCH₃, —CN and —SF₅; or a salt thereof.

In another embodiment the invention relates to compounds of general formula (Ic)

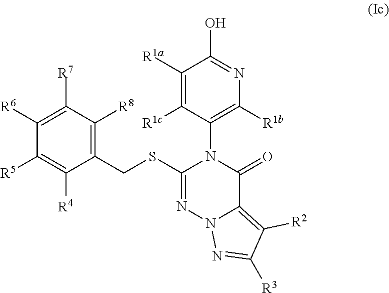

wherein R^{1c} is H; and wherein R^{1a} and R^{1b} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} is H or F; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} is H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of —H, -halogen, —C₁₋₄-alkyl, —C₁₋₄-haloalkyl; and wherein R³ is —H; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂C, —OCH₃, —CN and —SF₅; or a salt thereof.

In another embodiment the invention relates to compounds of general formula (Ia)

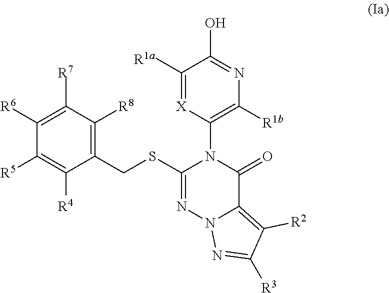

wherein X is N or C—R^{1c}; and wherein R^{1c} is H; and wherein R^{1a} and R^{1b} are H or F; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is H; and wherein R^{1a} and R^{1b} are H or F; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ia)
wherein X is N or C—R^{1c}; and wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ia)
wherein X is N or C-Ric; and wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂C, —OCH₃, —CN and —SF₅; or a salt thereof.

In another embodiment the invention relates to compounds of general formula (Ib)

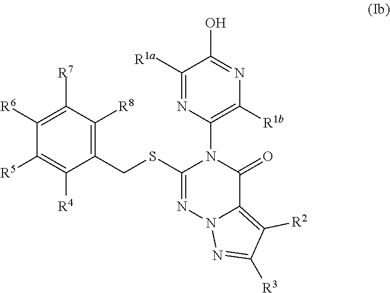

wherein R^{1a} and R^{1b} are H or F; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H or F; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ib)
wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂C, —OCH₃, —CN and —SF₅; or a salt thereof.

In another embodiment the invention relates to compounds of general formula (Ic)

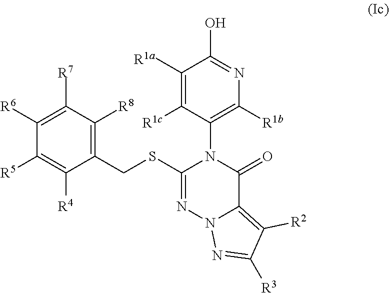

wherein R^{1c} is H; and wherein R^{1a} and R^{1b} are H or F; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} are H or F but provided that not both R^{1a} and R^{1b} are F simultaneously; and wherein R² is selected from among a group consisting of -cyclopropyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl; and wherein R³ is —H; and wherein R5 is H; and wherein R7 is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Ia)

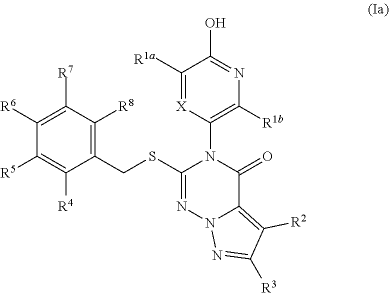

wherein X is N or C—R^{1c}; and wherein R^{1c} is H; and wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is H; and wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ia)
wherein X is N or C—R^{1c}; and wherein R^{1c} is H; and wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ia)
wherein X is N or C—R^{1c}; and wherein R^{1c} is H; and wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂C, —OCH₃, —CN and —SF₅; or a salt thereof.

In another embodiment the invention relates to compounds of general formula (Ib)

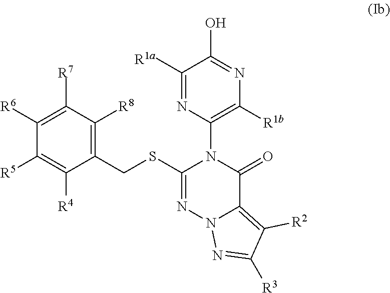

wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅. provided that at least two of R⁴, R⁵, R⁶, R⁷, R⁸ are hydrogen; or a salt thereof.

In another embodiment the invention relates to compounds of general formula (Ib)
wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ib)
wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to compounds of general formula (Ic)

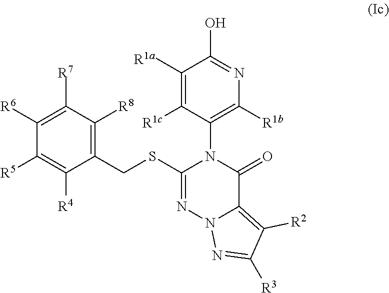

wherein R^{1c} is H; and wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} are H; and wherein R² is —H; wherein R³ is selected from among a group consisting of —F, —CH₃ and —CN; wherein R⁴, R⁵, R⁶, R⁷, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅, provided that at least two of R⁴, R⁵, R⁶, R⁷, R⁸ are hydrogen; or a salt thereof.

In another embodiment the invention relates to compounds of general formula (Ic)
wherein R^{1c} is H; and wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅; or a salt thereof.

In another embodiment the invention relates to compounds of general formula (Ic)
wherein R^{1c} is H; and wherein R^{1a} and R^{1b} are H; and wherein R² is —H; wherein R³ is selected from among a group consisting of —F, —CH₃ and —CN; and wherein R⁵ is H; and wherein R⁷ is H; and wherein R⁴, R⁶, R⁸ are, independently of each other, selected from among a group consisting of —H, —F, —Cl, —C₁₋₂-alkyl, -cyclopropyl, —CH═CH₂, —CH═CHF, —CH═CF₂, —C≡CH, —C≡C—CH₃, —CF₃, —CF₂H, —CF₂Cl, —OCH₃, —CN and —SF₅; or a salt thereof.

In another embodiment the invention relates to a compound selected from among a group consisting of

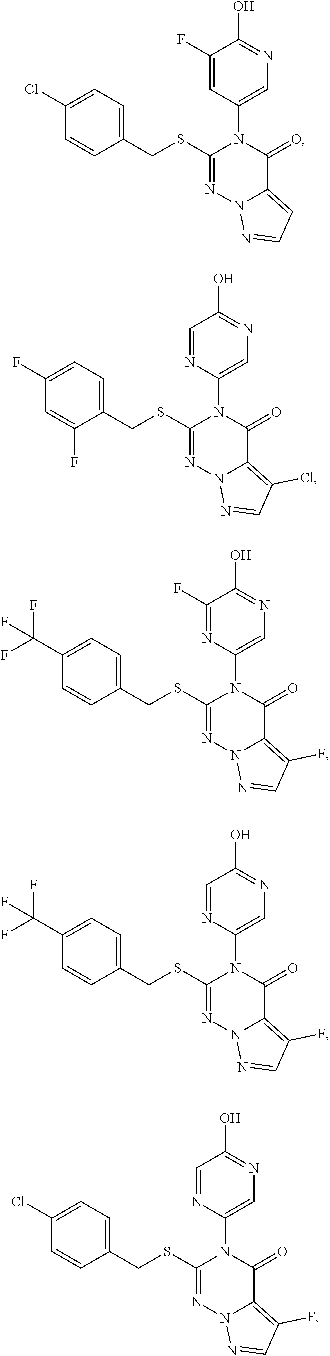

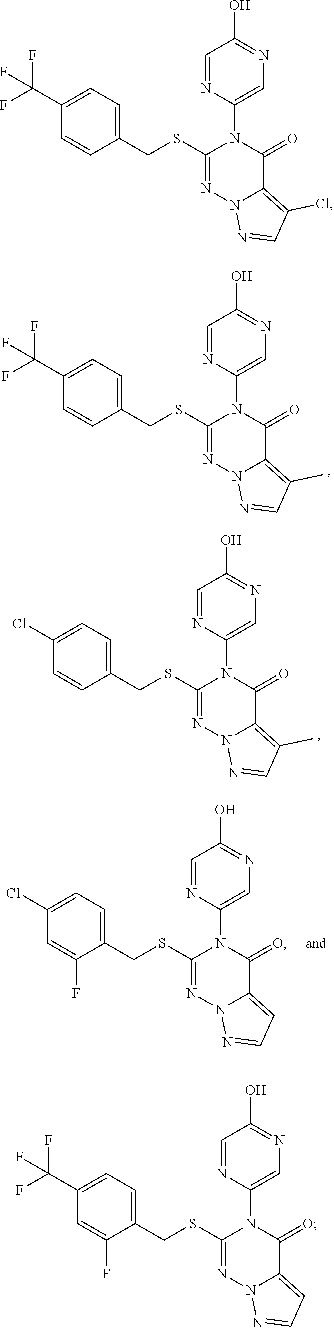

in its salt free form.

The following items are part of the invention:

1. A compound according to general formula (Ia)

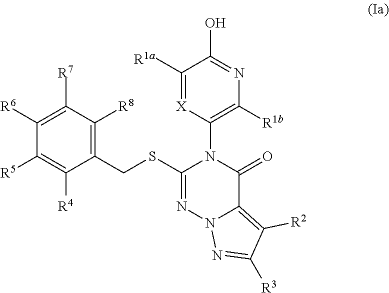

wherein
X is N or C—R^{1c};
R^{1c} is H;
R^{1a}, and R^{1b} are, independently of each other, H or F;
R² is selected from the group consisting of —H, -halogen, —C₁₋₄-alkyl, -cyclopropyl, —C₁₋₄haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl;
R³ is selected from the group consisting of —H, —C₁₋₄-alkyl, —CN, and -halogen;
R⁴, R⁵, R⁶, R⁷ and R⁸ are, independently of each other, selected from the group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅;
or a salt thereof.

2. The compound according to item 1, wherein X is N, or a salt thereof.

3. The compound according to item 1, wherein X is C—R^{1c}, or a salt thereof.

4. The compound according to any one of items 1 to 3, wherein
R⁴, R⁵, R⁶, R⁷, and R⁸ are, independently of each other, selected from among a group consisting of H, —F, Cl, —C₁₋₂-alkyl, -cyclopropyl, —CH═CH₂, —CH═CHF, —CH═CF₂, —C≡CH, —C≡CCH₃, —CF₃, —CF₂H, —CF₂Cl, —OCH₃, —CN and —SF₅; or a salt thereof.

5. The compound according to any one of items 1 to 4, wherein
R² is selected from the group consisting of —H, -halogen, —C₁₋₄-alkyl and —C₁₋₄-haloalkyl; and R³ is H; or a salt thereof.

6. The compound according to any one of items 1 to 4, wherein
R² is selected from among a group consisting of -cyclopropyl, —CN, —C₂₋₄-alkyn-1-yl, —C₂₋₄-alken-1-yl, —C₂₋₄-haloalken-1-yl, —O—C₁₋₄-alkyl, —CO₂C₁₋₄-alkyl, —C(═O)NH₂, —C(═O)NHC₁₋₄-alkyl, —NH₂, —NO₂, —C₁₋₄-alkyl-OH, —C₁₋₄-alkyl-O—C₁₋₄-alkyl, —C(═O)C₁₋₄-alkyl, —C(═O)H, —CH═NOH, —CH═NOCH₃, phenyl, 2-pyridyl, 3-pyridyl, and 1-methyl-pyrazol-3-yl; and
R³ is H; or a salt thereof.

7. The compound according to any one of items 1 to 4, wherein
R² is H; and
R³ is selected from the group consisting of —F, —CH₃ and —CN; or a salt thereof.

8. The compound according to any one of items 1 to 4, wherein
R^{1a} and R^{1b} is H;
R² is H; and
R³ is selected from the group consisting of —F, —CH₃ and —CN; or a salt thereof.

### Biological Assays

Assay to determine inhibition of NOX4

### Cell Line Generation for NOX4 Inhibition Assay

The synthetic gene phNOX4_DNA3_1_Zeo (Accession: AAF68973) is assembled from synthetic oligonucleotides and/or PCR products. The fragment is cloned into pcDNA3.1_Zeo_A011 using NheI and XhoI cloning sites. The plasmid DNA is purified from transformed bacteria and its concentration is determined by UV spectroscopy. The final construct is verified by sequencing. The plasmid is transfected via electroporation (Amaxa electroporation device) in combination with Nucleofector Kit V. Selection of NOX4 overexpressing genes is achieved using selection antibiotic Zeocin.

### NOX4 Inhibition Assay (HyPerBlu™)

The inhibitory activity of the example compounds of the invention is determined using the following procedure:

NOX4 inhibition is assessed utilizing HEK293 cells stably overexpressing human NOX4 (hNOX4), generating constitutively high levels of hydrogen peroxide (H₂O₂). Cells are cultured in the Dulbecco's Modified Eagle Medium (DMEM) media containing 4.5 g/L glucose supplemented with 10% fetal calf serum and 250 μg/ml Zeocin in an incubator at 37° C. with 5% CO₂. For the assay, cells are seeded in 384 well plates. After 24 h cells are washed and treated with several concentrations (10 nM-30 μM) of the test compounds (diluted in DMSO) or 100 μM diphenyleneiodonium chloride (DPI, used as positive control) in assay buffer consisting of phosphate-buffered saline (PBS) with 4-(2-hydroxyethyl)-1-piperazineethanesulfonic acid (HEPES), and incubated for 2 hours at 24° C. in a humidified incubator.

After incubation Lumigen HyPerBlu™ is added and cells are incubated for additional 30 min at 24° C. in a humidified incubator. Next, luminescence is measured using an Envision Multimode Plate reader to determine levels of produced H₂O₂ in the wells. Results are visible in Table 1.

TABLE 1

| Example | IC₅₀ (NOX4) |
|---|---|
| Number | [nM] |
| 1 | 78 |
| 2 | 339 |
| 3 | 86 |
| 4 | 299 |
| 5 | 135 |
| 6 | 786 |
| 7 | 240 |
| 8 | 97 |
| 9 | 57 |
| 10 | 79 |
| 11 | 71 |
| 12 | 67 |
| 13 | 207 |
| 14 | 272 |
| 15 | 66 |
| 16 | 32 |
| 17 | 303 |
| 18 | 170 |
| 19 | 60 |
| 20 | 46 |
| 21 | 83 |
| 22 | 350 |
| 23 | 48 |
| 24 | 135 |
| 25 | 73 |
| 26 | 122 |
| 27 | 97 |
| 28 | 42 |
| 29 | 80 |
| 30 | 83 |
| 31 | 67 |
| 32 | 93 |
| 33 | 45 |
| 34 | 28 |
| 35 | 28 |
| 36 | 91 |
| 37 | 112 |
| 38 | 46 |
| 39 | 41 |
| 40 | 24 |
| 41 | 107 |
| 42 | 26 |
| 43 | 119 |
| 44 | 45 |
| 45 | 14 |
| 46 | 45 |
| 47 | 20 |
| 48 | 44 |
| 49 | 35 |
| 50 | 49 |
| 51 | 58 |
| 52 | 70 |
| 53 | 36 |
| 54 | 33 |
| 55 | 37 |
| 56 | 29 |
| 57 | 30 |
| 58 | 43 |
| 59 | 32 |
| 60 | 35 |
| 61 | 28 |
| 62 | 38 |
| 63 | 40 |
| 64 | 220 |
| 65 | 161 |
| 66 | 68 |
| 67 | 33 |
| 68 | 46 |
| 69 | 59 |
| 70 | 36 |
| 71 | 35 |
| 72 | 72 |
| 73 | 122 |
| 74 | 69 |
| 75 | 64 |
| 76 | 31 |
| 77 | 32 |
| 78 | 43 |
| 79 | 123 |
| 80 | 45 |
| 81 | 36 |
| 82 | 134 |
| 83 | 70 |
| 84 | 63 |
| 85 | 66 |
| 86 | 65 |
| 87 | 65 |
| 88 | 48 |
| 89 | 56 |
| 90 | 36 |
| 91 | 16 |
| 92 | 26 |
| 93 | 25 |
| 94 | 28 |
| 95 | 18 |
| 96 | 11 |
| 97 | 13 |
| 98 | 36 |
| 99 | 38 |
| 100 | 124 |
| 101 | 20 |
| 102 | 47 |
| 103 | 43 |
| 104 | 44 |
| 105 | 15 |
| 106 | 87 |
| 107 | 96 |
| 108 | 64 |
| 109 | 80 |
| 110 | 59 |
| 111 | 37 |
| 112 | 52 |
| 113 | 56 |
| 114 | 48 |
| 115 | 120 |
| 116 | 166 |
| 117 | 88 |
| 118 | 60 |
| 119 | 77 |
| 120 | 267 |
| 121 | 210 |
| 122 | 182 |
| 123 | 237 |
| 124 | 311 |
| 125 | 100 |
| 126 | 133 |
| 127 | 109 |
| 128 | 91 |
| 129 | 78 |
| 130 | 115 |
| 131 | 75 |
| 132 | 228 |
| 133 | 36 |
| 134 | 105 |
| 135 | 55 |
| 136 | 141 |
| 137 | 77 |
| 138 | 101 |
| 139 | 48 |
| 140 | 132 |
| 141 | 96 |
| 142 | 59 |
| 143 | 45 |
| 144 | 32 |
| 145 | 39 |
| 146 | 57 |
| 147 | 85 |
| 148 | 59 |
| 149 | 32 |
| 150 | 55 |
| 151 | 73 |
| 152 | 102 |
| 153 | 86 |
| 154 | 86 |
| 155 | 243 |
| 156 | 247 |
| 157 | 360 |
| 158 | 31 |
| 159 | 52 |
| 160 | 55 |
| 161 | 119 |
| 162 | 134 |
| 163 | 55 |
| 164 | 150 |
| 165 | 244 |
| 166 | 175 |
| 167 | 280 |

### Assays to Determine the Selectivity Against NOX1, NOX2, NOX3, NOX5

### Cell Line Generation

Plasmids are purchased from Vectorbuilder.
for HEK-hNOX1 all necessary subunits are cloned accordingly:
   pLV-CMV-hNOX1-puro (NM_007052.5)
   pLV-CMV-hNOXO1-blas (NM_172168.3)
   pLV-CMV-hp22phox-hygro (NM_000101.4)
   pLV-CMV-hNOXA1-neo (NM_006647.2)
for HEK-hNOX3
   pLV-CMV-hNOX3-puro (NM_015718.3)

3rd generation lentiviral particles are produced in HEK293 suspension cells by transient transfection of three helper and expression plasmids. Crucial lentivirus supernatants are harvested, clarified by filtration and concentrated by precipitation. Lentivirus titers are determined in transducing units in (TU/mL) by transduction of HT1080 cells and colony forming assay. Parental HEK293 cells, purchased from CLS GmbH, are transduced with replication-incompetent 3rd generation lentiviral particles (MOI 2) and expanded for at least 2 weeks before cryopreservation of tested lots. During cultivation, medium is changed three times per week and cells are sub-cultured at least once per week. Selection antibiotics are added according to the plasmids (1 μg/ml puromycin, 1.5 μg/ml blasticidin, 200 μg/ml G418 and 100 μg/ml hygromycin).

### For HEK-hNOX5

The synthetic gene hNOX5 (Accession: Q96PH1) is cloned into pcDNA3.1_Zeo. The plasmid DNA is purified from transformed bacteria and its concentration is determined by UV spectroscopy. The final construct is verified by sequencing. The plasmid is transfected via electroporation (Amaxa electroporation device) in combination with Nucleofector Kit V. Selection of NOX5 overexpressing genes is achieved using selection antibiotic Zeocin

### NOX1 Inhibition Assay (L-012)

Evaluation of the NOX4 inhibitors on NOX1 activity is assessed in HEK293 cells stably overexpressing human NOX1 (hNOX1).

Cells are cultured in the DMEM media containing 4.5 g/L glucose supplemented with 10% fetal calf serum (FCS), Puromycin 1 μg/mL, Blasticidin 1.5 μg/mL, Geneticin (G418) 200 μg/mL and Hygromycin 100 μg/mL in an incubator at 37° C. with 5% CO₂. For the assay cells are seeded in 384 well plates in DMEM media with 10% FCS. After 24 h, cells are washed, treated for 30 mins with several concentrations (30 nM-100 μM) of test compounds and then stimulated with phorbol myristate acetate (PMA) at 1 μM to induce NOX1-dependent ROS production (except wells used as a positive control). Lastly, 8-amino-5-chloro-2,3-dihydro-7-phenyl-pyrido[3,4-d]pyridazine-1,4-dione sodium salt (L-012, CAS 143556-24-5) is added at 400 μM to all wells, and cells are incubated in an incubator for an additional 3 h. Afterwards luminescence is measured with a SpectraMax Paradigm Microplate Reader. Data can be found in table 2.

### NOX2 Inhibition Assay (L-012)

Evaluation of the NOX4 inhibitors on NOX2 activity is assessed in human blood-derived granulocytes.

Different concentrations of the test compounds (30 nM-100 μM), DMSO (as a negative control) or diphenyleneiodonium chloride (DPI; 30 μM, as a positive control), prepared in assay buffer (PBS containing CaCl2 and MgCl2+0.1% bovine serum albumin (BSA)), are placed in 384 well plates. Afterwards, freshly isolated granulocytes are added to the wells containing the compounds in assay buffer and incubated for 1 h at 37° C. Next, a solution of N-formylmethionine-leucyl-phenylalanine (fMLP; 730 nM), phorbol myristate acetate (PMA, 100 nM) (stimuli of ROS production) and L-012 (200 uM, ROS indicator) is added to the wells and the cells are incubated for additional 30 mins at 37° C. Afterwards luminescence is measured in a SpectraMax M5 microplate reader. Data can be found in table 2.

### NOX3 Inhibition Assay (L-012)

Evaluation of the NOX4 inhibitors on NOX3 activity is assessed in HEK293 cells stably overexpressing human NOX3 (hNOX3).

Cells are cultured in the DMEM media containing 4.5 g/L glucose supplemented with 10% fetal calf serum (FCS), Puromycin 1 μg/mL and Blasticidin 1.5 μg/mL in an incubator at 37° C. with 5% CO₂. Cells are seeded in 384 well plates for 2 days. For the assay cells are washed and treated with several concentrations (10 nM-30 μM) of test compound (diluted in DMSO) or 100 μM DPI (as a positive control in assay buffer consisting of PBS), and incubated for 2 hours at 24° C. in a humidified incubator. Cells are then stimulated with PMA at 0.1 μM to induce NOX3-dependent ROS production. Finally, L-012 is added at 100 μM and incubated for 2 hours at 24° C. in a humidified incubator. Luminescence is measured with an PHERAstar multimode reader. Data can be found in table 2.

### NOX5 Inhibition Assay (L-012)

Evaluation of the NOX4 inhibitors on NOX5 activity is assessed in HEK293 cells stably overexpressing human NOX5 (hNOX5).

Cells are cultured in the DMEM media containing 4.5 g/L glucose supplemented with 10% fetal calf serum and 250 μg/ml Zeocin in an incubator at 37° C. with 5% CO₂. Cells are seeded in 384 well plates over-night. For the assay cells are washed and treated with several concentrations (10 nM-30 μM) of test compounds (diluted in DMSO and added to assay buffer consisting of PBS) or 100 μM DPI as a positive control and incubated for 2 hours at 24° C. in a humidified incubator. Cells are then stimulated with PMA at 0.1 μM to induce NOX5-dependent ROS production. Finally, L-012 is added at 100 μM and incubated for 2 hours at 24° C. in a humidified incubator. Luminescence is measured with an PHERAstar multimode reader. Data can be found in table 2.

TABLE 2

| Example | IC₅₀ (NOX1) | IC₅₀ (NOX2) | IC₅₀ (NOX3) | IC₅₀ (NOX5) |
|---|---|---|---|---|
| Number | [μM] | [μM] | [μM] | [μM] |
| 1 | 84 | >100 | >100 | >100 |
| 2 | N/A | N/A | >100 | >100 |
| 5 | 22 | >100 | 47 | 17 |
| 6 | 77 | >100 | 97 | >100 |
| 7 | >100 | >100 | >100 | >100 |
| 8 | 55 | >100 | >100 | >100 |
| 9 | >100 | >100 | >100 | >100 |
| 10 | 79 | >100 | 54 | 78 |
| 11 | >100 | >100 | 70 | >100 |
| 12 | >100 | >100 | >100 | >100 |
| 13 | 73 | >100 | 55 | 89 |
| 14 | >100 | >100 | >100 | 28 |
| 15 | 38 | >100 | 18 | 17 |
| 16 | 24 | >100 | 42 | 32 |
| 17 | 51 | >100 | >100 | >100 |
| 18 | 46 | >100 | 80 | >100 |
| 19 | 29 | >100 | 44 | 21 |
| 20 | >100 | >100 | 58 | 65 |
| 21 | 34 | >100 | 25 | 12 |
| 22 | 3.5 | 25.1 | 22 | 14 |
| 23 | >100 | >100 | 61 | 29 |
| 24 | 68 | >100 | >100 | 94 |
| 25 | 3.6 | >100 | 23 | 14 |
| 26 | N/A | N/A | 61 | 83 |
| 27 | 69 | >100 | >100 | >100 |
| 28 | 68 | >100 | 53 | 89 |
| 29 | >100 | >100 | 19 | >100 |
| 30 | >100 | >100 | >100 | >100 |
| 31 | >100 | >100 | 28 | >100 |
| 32 | >100 | >100 | 13 | >100 |
| 33 | >100 | >100 | 13 | 90 |
| 34 | >100 | >100 | >100 | >100 |
| 35 | 28 | 77 | 35 | 66 |
| 36 | >100 | N/A | N/A | N/A |
| 37 | >100 | >100 | >100 | >100 |
| 38 | 54 | >100 | 44 | >100 |
| 39 | 29 | 86 | 14 | 33 |
| 40 | >100 | >100 | 13 | 52 |
| 41 | N/A | >100 | >100 | >100 |
| 42 | >100 | >100 | >100 | >100 |
| 43 | >100 | >100 | >100 | >100 |
| 44 | >100 | N/A | >100 | >100 |
| 45 | >100 | >100 | >100 | >100 |
| 46 | >100 | >100 | 20 | >100 |
| 47 | 68 | >100 | 56 | 70 |
| 48 | >100 | >100 | >100 | >100 |
| 49 | >100 | >100 | >100 | >100 |
| 50 | >100 | >100 | >100 | >100 |
| 51 | 28 | >100 | 23 | 35 |
| 52 | 14 | >100 | 32 | 33 |
| 53 | >100 | >100 | >100 | >100 |
| 54 | >100 | >100 | 18 | 25 |
| 55 | >100 | >100 | 72 | 110 |
| 56 | 29 | >100 | 29 | 49 |
| 57 | 38 | >100 | 34 | >100 |
| 58 | >100 | 49 | 23 | 33 |
| 59 | >100 | >100 | 54 | 52 |
| 60 | 41 | >100 | 84 | >100 |
| 61 | 26 | >100 | 23 | 20 |
| 62 | 23 | N/A | 13 | 15 |
| 63 | 25 | >100 | 28 | 65 |
| 64 | >100 | >100 | 22 | 24 |
| 65 | >100 | >100 | >100 | >100 |
| 66 | >100 | >100 | 24 | 19 |
| 67 | 39 | >100 | >100 | >100 |
| 68 | >100 | >100 | 35 | 84 |
| 69 | 37 | >100 | >100 | >100 |
| 70 | 93 | >100 | >100 | >100 |
| 71 | 66 | >100 | 53 | 35 |
| 72 | 29 | >100 | >100 | >100 |
| 73 | 73 | >100 | 26 | 71 |
| 74 | 73 | >100 | >100 | >100 |
| 75 | >100 | >100 | >100 | >100 |
| 76 | >100 | >100 | >100 | >100 |
| 77 | 27 | >100 | 38 | 40 |
| 78 | >100 | >100 | >100 | 68 |
| 79 | 49 | >100 | 25 | 54 |
| 80 | >100 | >100 | >100 | >100 |
| 81 | >100 | >100 | 33 | 34 |
| 82 | 30 | >100 | >100 | >100 |
| 83 | 88 | >100 | >100 | >100 |
| 84 | 29 | 88 | 20 | 17 |
| 85 | >100 | >100 | >100 | >100 |
| 86 | >100 | >100 | 29 | 37 |
| 87 | 26 | >100 | >100 | >100 |
| 88 | >100 | >100 | >100 | >100 |
| 89 | >100 | >100 | >100 | >100 |
| 90 | 80 | N/A | N/A | N/A |
| 91 | N/A | N/A | 88 | 84 |
| 92 | 73 | >100 | 48 | 48 |
| 93 | >100 | >100 | >100 | 89 |
| 94 | >100 | >100 | 20 | 88 |
| 95 | 69 | >100 | >100 | 72 |
| 96 | >100 | >100 | >100 | >100 |
| 97 | 19 | >100 | 31 | 26 |
| 98 | 82 | >100 | 4 | >100 |
| 99 | 67 | >100 | 10 | 91 |
| 100 | >100 | >100 | 53 | 39 |
| 101 | 67 | >100 | 73 | 44 |
| 102 | >100 | >100 | 15 | >100 |
| 103 | >100 | >100 | 8 | >100 |
| 104 | >100 | >100 | >100 | >100 |
| 105 | >100 | >100 | >100 | >100 |
| 106 | 51 | >100 | 100 | 54 |
| 107 | 68 | >100 | 52 | 23 |
| 108 | 42 | >100 | 70 | 18 |
| 109 | 71 | >100 | >100 | 96 |
| 110 | 80 | >100 | 55 | 46 |
| 111 | 30 | >100 | 68 | 83 |
| 112 | 70 | >100 | >100 | >100 |
| 113 | 74 | >100 | >100 | >100 |
| 114 | >100 | >100 | >100 | 61 |
| 115 | N/A | N/A | 54 | 27 |
| 116 | N/A | N/A | >100 | 81 |
| 117 | 60 | >100 | >100 | 72 |
| 118 | 22 | >100 | 60 | 42 |
| 119 | N/A | N/A | >100 | 70 |
| 120 | >100 | N/A | 4 | >100 |
| 121 | 31 | 33 | 47 | 7 |
| 122 | 67 | >100 | 43 | 36 |
| 123 | >100 | N/A | N/A | N/A |
| 125 | >100 | >100 | >100 | >100 |
| 126 | >100 | >100 | >100 | >100 |
| 127 | >100 | >100 | >100 | >100 |
| 128 | 34 | >100 | >100 | >100 |
| 129 | 74 | >100 | >100 | 39 |
| 130 | N/A | >100 | 76 | 45 |
| 131 | >100 | >100 | >100 | >100 |
| 132 | >100 | >100 | >100 | >100 |
| 133 | 69 | >100 | 5 | 70 |
| 135 | 68 | >100 | >100 | 110 |
| 137 | 61 | >100 | 46 | 44 |
| 139 | >100 | >100 | 29 | >100 |
| 141 | 70 | >100 | >100 | >100 |
| 142 | >100 | >100 | >100 | >100 |
| 143 | >100 | >100 | >100 | 68 |
| 144 | >100 | >100 | >100 | >100 |
| 145 | >100 | >100 | >100 | 87 |
| 146 | >100 | >100 | >100 | 84 |
| 147 | 19 | N/A | >100 | 56 |
| 148 | 6 | >100 | 11 | 8 |
| 149 | 55 | >100 | 93 | 62 |
| 150 | >100 | >100 | >100 | >100 |
| 151 | 63 | >100 | >100 | 28 |
| 152 | 16 | >100 | 46 | 26 |
| 153 | >100 | >100 | >100 | 100 |
| 154 | >100 | >100 | >100 | >100 |
| 155 | N/A | N/A | >100 | >100 |
| 158 | 61 | >100 | >100 | >100 |
| 159 | >100 | >100 | >100 | >100 |
| 160 | >100 | >100 | >100 | >100 |
| 161 | >100 | >100 | >100 | >100 |
| 162 | >100 | >100 | >100 | >100 |
| 163 | N/A | N/A | >100 | >100 |
| 164 | 81 | >100 | >100 | >100 |
| 165 | 74 | >100 | >100 | 61 |
| 166 | >100 | >100 | >100 | >100 |
| 167 | >100 | >100 | >100 | >100 |

N/A = not available

### Evaluation of Hepatocyte Clearance

The metabolic degradation of the test compound is assayed in a hepatocyte suspension. Hepatocytes (cryopreserved) are incubated in Dulbecco's modified eagle medium (supplemented with 3.5 μg glucagon/500 mL, 2.5 mg insulin/500 mL and 3.75 mg/500 mL hydrocortisone) containing 5% human serum.

Following a 30 min preincubation in an incubator (37° C., 10% CO₂) 5 μl of test compound solution (80 μM; from 2 mM in DMSO stock solution diluted 1:25 with medium) are added into 395 μl hepatocyte suspension (cell density in the range 0.25-5 Mio cells/mL, typically 1 Mio cells/mL; final concentration of test compound 1 μM, final DMSO concentration 0.05%).

The cells are incubated for six hours (incubator, orbital shaker) and samples (25 μl) are taken at 0, 0.5, 1, 2, 4 and 6 hours. Samples are transferred into acetonitrile and pelleted by centrifugation (5 min). The supernatant is transferred to a new 96-deepwell plate, evaporated under nitrogen and resuspended.

Decline of parent compound is analyzed by HPLC-MS/MS.

CLᵢₙₜ is calculated as follows:

$$CL\_INTRINSIC = Dose / AUC = ( C 0 / CD ) / ( AUD + clast / k ) \times 1000 / 60.$$

C0: initial concentration in the incubation [μM], CD: cell density of vital cells [10⁶ cells/mL], AUD: area under the data [μM×h], clast: concentration of last data point [μM], k: slope of the regression line for parent decline [h−1].

The calculated in vitro hepatic intrinsic clearance can be scaled up to the intrinsic in vivo hepatic clearance and used to predict hepatic in vivo blood clearance (CL) by the use of a liver model (well stirred model).

$$CL\_INTRINSIC \_INVIVO [ ml / min / kg ] = ( CL\_INTRINSIC [ μL / min / {10}^{6} cells ] \times hepatocellularity [ {10}^{6} cells / g liver ] \times liver factor [ g / kg bodyweight ] ) / 1000$$

$$CL [ ml / min / kg ] = CL\_INTRINSIC \_INVIVO [ ml / min / kg ] \times hepatic blood flow [ ml / min / kg ] / ( CL\_INTRINSIC \_INVIVO [ ml / min / kg ] + hepatic blood flow [ ml / min / kg ] )$$

$$Qh [ \% ] = CL [ ml / min / kg ] / hepatic blood flow [ ml / min / kg ] )$$

Hepatocellularity, human: 120×10⁶ cells/g liver
Liver factor, human: 25.7 g/kg bodyweight
Blood flow, human: 21 ml/(min×kg)

The resulting data are visible in table 3.

| Example | Qh |
|---|---|
| Number | [%] |
| 8 | 11 |
| 9 | 24 |
| 10 | 27 |
| 11 | 25 |
| 12 | 23 |
| 15 | 48 |
| 16 | 21 |
| 18 | 46 |
| 19 | 49 |
| 20 | 32 |
| 21 | 15 |
| 23 | 32 |
| 25 | 65 |
| 26 | 21 |
| 27 | 15 |
| 28 | 15 |
| 29 | 25 |
| 30 | 15 |
| 31 | 22 |
| 32 | 15 |
| 33 | 18 |
| 34 | 12 |
| 35 | 20 |
| 37 | 24 |
| 38 | 20 |
| 39 | 24 |
| 40 | 20 |
| 41 | 20 |
| 42 | 22 |
| 43 | 24 |
| 44 | 9 |
| 45 | 33 |
| 46 | 32 |
| 47 | 29 |
| 48 | 34 |
| 49 | 38 |
| 50 | 21 |
| 51 | 20 |
| 52 | 27 |
| 53 | 27 |
| 54 | 21 |
| 55 | 33 |
| 56 | 40 |
| 57 | 34 |
| 58 | 37 |
| 59 | 33 |
| 60 | 38 |
| 61 | 34 |
| 62 | 36 |
| 63 | 47 |
| 64 | 14 |
| 67 | 46 |
| 68 | 32 |
| 69 | 38 |
| 70 | 15 |
| 71 | 34 |
| 72 | 27 |
| 74 | 37 |
| 76 | 32 |
| 77 | 36 |
| 78 | 31 |
| 80 | 38 |
| 81 | 43 |
| 83 | 31 |
| 84 | 40 |
| 85 | 31 |
| 86 | 17 |
| 88 | 57 |
| 89 | 38 |
| 90 | 52 |
| 91 | 65 |
| 92 | 25 |
| 93 | 28 |
| 94 | 12 |
| 95 | 12 |
| 96 | 11 |
| 97 | 11 |
| 98 | 20 |
| 99 | 24 |
| 101 | 18 |
| 102 | 21 |
| 103 | 26 |
| 104 | 16 |
| 105 | 21 |
| 106 | 20 |
| 107 | 20 |
| 108 | <4 |
| 109 | 45 |
| 110 | <4 |
| 111 | 10 |
| 112 | 29 |
| 113 | 25 |
| 114 | 23 |
| 115 | 23 |
| 116 | 26 |
| 117 | 24 |
| 118 | 24 |
| 119 | 25 |
| 125 | 4 |
| 126 | 28 |
| 127 | 8 |
| 128 | 34 |
| 129 | 36 |
| 130 | 6 |
| 131 | 9 |
| 132 | 22 |
| 133 | 31 |
| 135 | 5 |
| 137 | 9 |
| 139 | <4 |
| 141 | 17 |
| 142 | 10 |
| 143 | <4 |
| 144 | 6 |
| 145 | 6 |
| 146 | <4 |
| 147 | 14 |
| 148 | <4 |
| 149 | 4 |
| 150 | <4 |
| 151 | 19 |
| 152 | 11 |
| 153 | 17 |
| 154 | 31 |
| 158 | <4 |
| 159 | <4 |
| 160 | 38 |
| 161 | 16 |
| 162 | <4 |
| 163 | 7 |
| 166 | 10 |

### Method of Treatment

The present invention is directed to compounds of general formula (I) which are useful in the prevention and/or treatment of a disease and/or condition associated with or modulated by NOX4 activity, including but not limited to the treatment and/or prevention of chronic liver diseases, portal hypertension, viral infections, cancer, interstitial lung diseases, retinopathies, acute and chronic inflammation as well as fibrotic diseases. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

The compounds of general formula (I) are useful for the prevention and/or treatment of: vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza, ischemic and hemorrhagic stroke.

Accordingly, the present invention relates to a compound of general formula (I) for use as a medicament.

Furthermore, the present invention relates to the use of a compound of general formula (I) for the treatment and/or prevention of a disease and/or condition associated with or modulated by NOX4 activity.

Furthermore, the present invention relates to the use of a compound of general formula (I) for the treatment and/or prevention of chronic liver diseases, viral infections, cancer, interstitial lung diseases, retinopathies, acute and chronic inflammation as well as fibrotic diseases.

Particularly, the pharmaceutical compositions of the invention are suitable for the therapy of interstitial lung diseases, e.g. idiopathic pulmonary disease, and may also be suitable for the therapy of fibrotic, allergic and inflammatory diseases.

Furthermore, the present invention relates to the use of a compound of general formula (I) for the treatment and/or prevention of: vascular inflammation, atherosclerosis, interstitial lung diseases, e.g. idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, and head and neck tumors), systemic sclerosis, inflammatory bowel disease, Duchenne muscular dystrophy, COVID-19, acute respiratory distress syndrome, influenza, pulmonary hypertension, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.

In a further aspect the present invention relates to a compound of general formula (I) for use in the treatment and/or prevention of above mentioned diseases and conditions.

In a further aspect the present invention relates to the use of a compound of general formula (I) for the preparation of a medicament for the treatment and/or prevention of above mentioned diseases and conditions.

In a further aspect of the present invention the present invention relates to methods for the treatment or prevention of above mentioned diseases and conditions, which method comprises the administration of an effective amount of a compound of general formula (I) to a human being.

The dose range of the compounds of general formula (I) applicable per day is usually from 0.00001 to 100 mg per kg body weight, for example from 0.00001 to 10 mg per kg body weight of the patient. Each dosage unit may conveniently contain from 0.001 to 1000 mg, for example from 0.001 to 100 mg.

The actual pharmaceutically effective amount or therapeutic dosage will usually depend on factors known by those skilled in the art such as age and weight of the patient, route of administration and severity of disease. In any case the compounds will be administered at dosages and in a manner which allows a pharmaceutically effective amount to be delivered based upon patient's unique condition.

### Pharmaceutical Composition

Suitable preparations for administering the compounds of formula (I) will be apparent to those with ordinary skill in the art and include for example tablets, pills, capsules, suppositories, lozenges, troches, solutions, syrups, elixirs, sachets, injectables, inhalables and powders etc.

Suitable tablets may be obtained, for example, by mixing one or more compounds according to formula (I) with known excipients, for example inert diluents, carriers, disintegrants, adjuvants, surfactants, binders and/or lubricants.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with chronic liver diseases, viral infections, cancer, interstitial lung diseases, retinopathies, acute and chronic inflammation as well as fibrotic diseases.

According to another embodiment, the additional therapeutic agent is selected from the group of therapeutic agents useful in the treatment of diseases or conditions described hereinbefore, in particular associated with vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza, ischemic and hemorrhagic stroke.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of antifibrotics (e.g. Ofev, PDE4i); of immunotherapeutics (e.g. PD-1, aCTLA-4); of T cell engagers; of sGC activators; of ATX-inhibitors; of SGLT2 inhibitors (e.g. dapagliflozin, empagliflozin); of THRb inhibitors; of GLP1 agonists and GLP1 agonist combinations; of FGF-analogs, such as FGF21 or FGF19; of KRAS-G12C-inhibitors (e.g. sotorasib); of KRAS-G12D-inhibitors; of MDM2-p53-antagonists; of Her2-inhibitors; of platinum derivatives (e.g. cisplatin, oxaliplatin, carboplatin); of alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); of antimitotic agents (e.g. Vinca alkaloids e.g. vinblastine, vindesin, vinorelbin and vincristine); of taxanes such as paclitaxel, docetaxel, nab-paclitaxel (Abraxane); of angiogenesis inhibitors (e.g. tasquinimod, bevacizumab); of tubuline inhibitors; of DNA synthesis inhibitors; of PARP inhibitors; of topoisomerase inhibitors (e.g. epipodophyllotoxins such as for example etoposide and etopophos, teniposide, amsacrin, topotecan, irinotecan, mitoxantrone); of antimetabolites (e.g. methotrexate, raltitrexed, 5-fluorouracil (5-FU), capecitabine, floxuridine, gemcitabine, mercaptopurine, thioguanine, cladribine, pentostatin, cytarabine (ara C), fludarabine, combination of trifluridine and tipiracil (=TAS102)); of antitumor antibiotics (e.g. anthracyclins such as doxorubicin, doxil (pegylated liposomal doxorubicin hydrochloride), myocet (non-pegylated liposomal doxorubicin), daunorubicin, epirubicin and idarubicin, mitomycin-C, bleomycin, dactinomycin, plicamycin, streptozocin); of inhibitors of vascular endothelial growth factor. Furthermore, the compounds according to the present invention can be combined with a radiotherapy regime.

Therefore, in another aspect, this invention relates to the use of a compound according to the invention in combination with one or more additional therapeutic agents described hereinbefore and hereinafter for the treatment of diseases or conditions which may be affected or which are mediated by NOX4, in particular diseases or conditions as described hereinbefore and hereinafter.

In a further aspect this invention relates to a method for treating a disease or condition which can be influenced by the inhibition of NOX4 in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of NOX4 in a patient in need thereof.

In yet another aspect the present invention relates to a method for the treatment of a disease or condition mediated by NOX4 activity in a patient that includes the step of administering to the human patient, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the invention and their intermediates may be obtained using methods of synthesis which are known to the one skilled in the art and described in the literature of organic synthesis. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the. The terms “ambient temperature” and “room temperature” are used interchangeably and designate a temperature of about 20° C., e.g. between 19 and 24° C.

Intermediates II (Step 1, intermediates I→intermediates II) can be prepared by treating intermediates I with a suitable amination reagent, for example O-diphenylphosphinylhydroxylamine, O-(4-nitrobenzoyl)hydroxylamine, O-(2,4-dinitrophenyl)hydroxylamine, O-(mesitylsulfonyl)hydroxylamine (MSH), hydroxylamine-O-sulfonic acid, in combination with a suitable base for example lithium-bis-(trimethylsilyl)-amide or sodium hydride, in a suitable solvent, for example N,N-dimethylformamide or tetrahydrofuran (Scheme 1). Preferred reaction temperatures are between −10° C. and room temperature. Formation of the thiourea (Step 2, intermediates II→intermediates III) can be achieved by reacting intermediates H with a thioisocyanate either in the presence of a suitable base, such as triethylamine or N,N-diisopropylethylamine, or without base, in a suitable solvent, for example acetonitrile. Preferred reaction temperatures are between room temperature and 80° C. The cyclization (Step 3, intermediates III→intermediates IV) can be achieved by treating intermediates III with a suitable base, for example potassium tert-butoxide, in an appropriate solvent, for example acetonitrile or N,N-dimethylacetamide. Compounds according to the present invention V (step 4, intermediates IV→compounds of the invention V) can be prepared by reaction of intermediates IV with a benzyl halide (i.e. chloride or bromide) and a suitable base, such as N,N-diisopropylethylamine or triethylamine, or without base, in a suitable solvent, for example acetonitrile, N,N-dimethylacetamide, N,N-dimethylformamide, methanol or tetrahydrofuran.

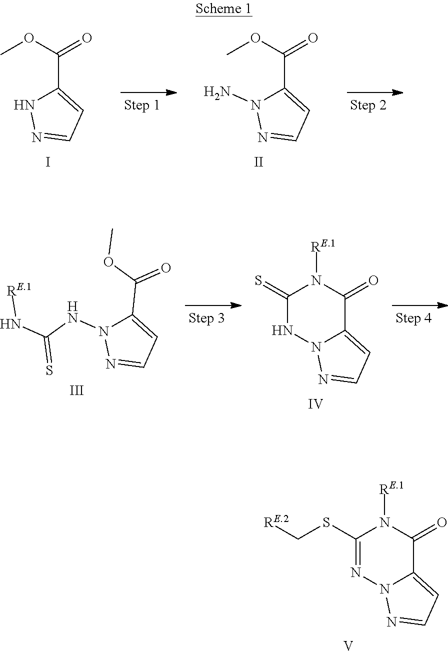

Alternatively, compounds of the present invention V can directly be obtained by combining step 2-4 in a one pot procedure. Intermediates III can be obtained by reacting intermediates II with a thioisocyanate-R^{E,1}—NH₂ in acetonitrile (Step 2, intermediates II→intermediates III) (Scheme 1). Preferred reaction temperatures are between room temperature and 80° C. The cyclization can be achieved by direct addition of potassium tert-butoxide to the reaction mixture (step 3, intermediates III→intermediates IV). Compounds according to the present invention V (step 4, intermediates IV→compounds of the invention V) can be prepared by addition of a benzyl halide (i.e. chloride or bromide) —R^{E,2}—CH₂—Z (Z is halide) and a suitable base, such as triethylamine, or without base to the reaction mixture.

### EXAMPLES

### Abbreviations

| | |
|---|---|
| ACN | acetonitrile |
| Aq. | aqueous |
| Boc | tert-butyloxycarbonyl |
| ° C. | degree celsius |
| CDI | carbonyl diimidazole |
| CyH/CH | cyclohexane |
| conc. | concentrated |
| DCM | dichloromethane |
| DIPEA | N,N-diisopropylethylamine |
| DMA | N,N-dimethylacetamide |
| DMF | N,N-dimethylformamide |
| DMP | Dess-Martin periodinane |
| DMSO | dimethyl sulfoxide |
| ESI-MS | electrospray ionisation mass spectrometry |
| EtOAc | ethyl acetate |
| EtOH | ethanol |
| ex | example |
| equiv | equivalent |
| FA | formic acid |
| h | hour |
| HATU | 1-[bis(dimethylamino)methylene]-1H-1,2,3-triazolo[4,5-b] |
| | pyridinium 3-oxid hexafluorophosphate |
| HCl | hydrochloric acid |
| HPLC | high performance liquid chromatography |
| Int. | intermediate |
| K₂CO₃ | potassium carbonate |
| K(OtBu) | potassium tert-butoxide |
| L | liter |
| LiOH*H₂O | lithium hydroxide monohydrate |
| M | molar (mol/L) |
| MeOH | methanol |
| MgSO₄ | magnesium sulphate |
| min | minute |
| mL | milliliter |
| MTBE | tert-butylmethylether |
| MW | molecular weight |
| NaOEt | sodium ethanolate |
| NH₃ | ammonia |
| PMB | para-methoxybenzyl |
| Prep. | preparative |
| RP | reversed phase |
| R_{f} | retardation factor |
| Rₜ | retention time |
| sat. | saturated |
| scCO₂ | supercritical CO₂ |
| SFC | supercritical fluid chromatography |
| TBTU | 2-(1H-benzotriazole-1-yl)-1,1,3,3-tetramethylaminium |
| | tetrafluoroborate |
| TCDI | 1,1-thiocarbonyldiimidazole |
| TEA | triethylamine |
| TFA | trifluoroacetic acid |
| TFAA | trifluoroacetic anhydride |
| THF | tetrahydrofuran |
| TBS | tert-butyldimethylsilyl |
| TMS | trimethylsilyl |
| TMS-Cl | trimethylsilyl chloride |
| XphosPdG3 | methanesulfonato(2-dicyclohexylphosphino-2′,4′,6′- |
| | tri-i-propyl-1,1′-bi-phenyl)(2′-amino-1,1′- |
| | biphenyl-2-yl)palladium(II) |
| XPhosPdG4 | methanesulfonato(2-dicyclohexylphosphino-2′,4′,6′- |
| | tri-i-propyl-1,1′-bi-phenyl)(2′-methylamino-1,1′- |
| | biphenyl-2-yl)palladium(II) |

### Preparation of Starting Materials

Unless stated otherwise, the starting materials are commercially available and used without further purification.

### Preparation of Intermediates

### Intermediate 1

### Methyl 1-amino-4-fluoro-1H-pyrazole-5-carboxylate

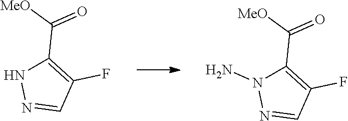 [SMILES: COC(=O)c1[nH]ncc1F ; COC(=O)c1c(F)cnn1N]

A solution of lithium-bis-(trimethylsilyl)-amide in tetrahydrofuran (1 M, 68.7 mL, 68.7 mmol, 1.10 equiv) is added dropwise to a mixture of methyl 4-fluoro-1H-pyrazole-5-carboxylate (9.00 g, 62.5 mmol, 1 equiv) in N,N-dimethylformamide (150 mL) at −10° C. After 15 min, a mixture of tetrahydrofuran (100 mL) and N,N-dimethylformamide (25 mL) is added followed by the dropwise addition of a solution of O-diphenylphosphinylhydroxylamine (17.5 g, 74.9 mmol, 1.20 equiv) in N,N-dimethylformamide (50 mL). After complete addition, more NN-dimethylformamide (25 mL) and tetrahydrofuran (100 mL) is added, and the mixture is allowed to warm to room temperature. After 18 h, the volatiles are removed under reduced pressure. The residue is dissolved in ethyl acetate and the organic phase is washed with saturated aqueous sodium chloride solution. The washed solution is dried over sodium sulfate and all volatiles are removed under reduced pressure, and the residue is purified by flash column chromatography (silica gel, gradient cyclohexane to cyclohexane/EtOAc 70:30) to provide the product.

| | |
|---|---|
| C₅H₆FN₃O₂ | (MW = 159.1 g/mol) |
| ESI | 160 [M + H]⁺ |
| Rₜ (HPLC) | 0.48 min (Z018_S04) |

The following compounds are prepared using procedures analogous to those described for intermediate 1, using appropriate starting materials. As is appreciated by those skilled in the art, the analogous examples may involve variations in general reaction conditions.

| Inter- | | | HPLC retention time |
|---|---|---|---|
| mediate | Structure | ESI-MS | [min] (method) |
| 2 | 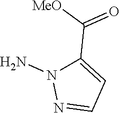 | 142 [M + H]⁺ | 0.45 (Z018_S04) |
| 4 | 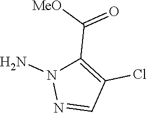 [SMILES: COC(=O)c1c(Cl)cnn1N] | 176/178 [M + H]⁺ | 0.61 (Z018_S04) |
| 5 | 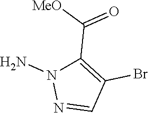 [SMILES: COC(=O)c1c(Br)cnn1N] | 220/222 [M + H]⁺ | 0.32 (X018_S03) |
| 6 | 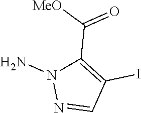 [SMILES: COC(=O)c1c(I)cnn1N] | 268 [M + H]⁺ | 0.52 (X018_S03) |
| 7 | 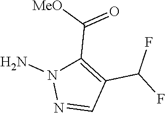 [SMILES: COC(=O)c1c(C(F)F)cnn1N] | 192 [M + H]⁺ | 0.59 (Z011_S03) |
| 8 | 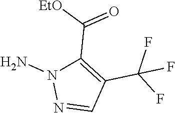 [SMILES: CCOC(=O)c1c(C(F)(F)F)cnn1N] | 224 [M + H]⁺ | 0.84 (Z018_S04) |
| 9 | 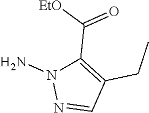 | 184 [M + H]⁺ | 0.45 (X012_S01) |
| 10 | 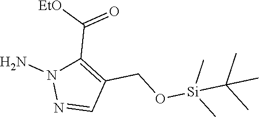 | 300 [M + H]⁺ | 0.71 (X012_S01) |
| 11 | 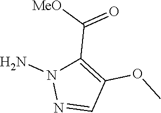 | 172 [M + H]⁺ | 0.53 (Z018_S04) |
| 12 | 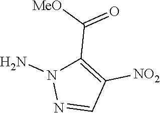 [SMILES: COC(=O)c1c([N+](=O)[O-])cnn1N] | 187 [M + H]⁺ | 0.60 (Z018_S04) |
| 13 | 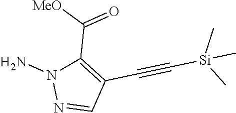 [SMILES: COC(=O)c1c(C#C[Si](C)(C)C)cnn1N] | 238 [M + H]⁺ | 0.89 (X018_S03) |
| 14 | 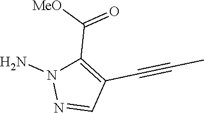 | 180 [M + H]⁺ | 0.50 (X018_S03) |
| 15 | 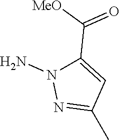 | 156 [M + H]⁺ | 0.28 (X012_S01) |
| 16 | 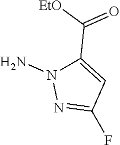 [SMILES: CCOC(=O)c1cc(F)nn1N] | 174 [M + H]⁺ | 0.35 (X012_S01) |
| 17 | 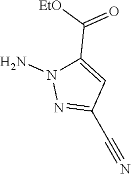 [SMILES: CCOC(=O)c1cc(C#N)nn1N] | 181 [M + H]⁺ | 0.35 (X012_S01) |
| 18 | 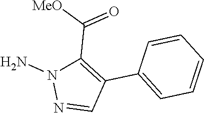 [SMILES: COC(=O)c1c(-c2ccccc2)cnn1N] | 218 [M + H]⁺ | 0.80 (Z018_S04) |
| 19 | 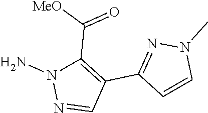 [SMILES: COC(=O)c1c(-c2ccn(C)n2)cnn1N] | 222 [M + H]⁺ | 0.59 (Z018_S04) |
| 20 | 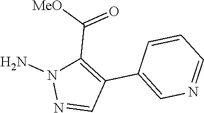 [SMILES: COC(=O)c1c(-c2cccnc2)cnn1N] | 219 [M + H]⁺ | 0.23 (Z018_S04) |

### Intermediate 21

### Ethyl 1-amino-4-cyano-1H-pyrazole-5-carboxylate

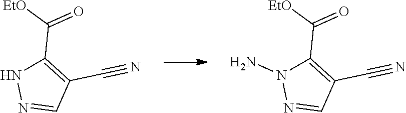 [SMILES: CCOC(=O)c1[nH]ncc1C#N ; CCOC(=O)c1c(C#N)cnn1N]

Sodium hydride (60% mineral oil dispersion, 1.62 g, 37.0 mmol, 1.30 equiv) is added to a solution of ethyl 4-cyano-1H-pyrazole-5-carboxylate (5.00 g, 28.8 mmol, 1 equiv) in N,N-dimethylformamide (25 mL) at −10° C. After 20 min, a solution of O-(4-nitrobenzoyl)hydroxylamine (7.86 g, 43.1 mmol, 1.50 equiv) N,N-dimethylformamide (25 mL) is added. After 1 h, a saturated, aqueous sodium chloride solution and ethyl acetate are added. The layers are separated, and the aqueous phase is extracted with ethyl acetate. The combined organic layers are washed with saturated aqueous sodium bicarbonate solution. The washed solution is dried over sodium sulfate, the dried solution is filtered, and the filtrate is concentrated under reduced pressure. The residue is purified by flash column chromatography (silica gel, gradient cyclohexane to cyclohexane/EtOAc 50:50) to provide the product.
C₇H₈N₄O₂ (MW=180.1 g/mol)
ESI 181[M+H]⁺
R: (HPLC) 0.63 min (Z018_S04)

### Intermediate 22

### Methyl 1-amino-4-methyl-1H-pyrazole-5-carboxylate

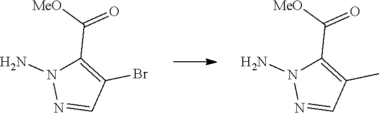

A mixture of methyl 1-amino-4-bromo-1H-pyrazole-5-carboxylate (intermediate 5, 1.00 g, 4.55 mmol, 1 equiv), trimethylboroxine (50% in THF, 2.00 mL, 7.09 mmol, 1.60 equiv), XPhosPdG4 (200 mg, 232 μmol, 0.100 equiv), cesium carbonate (3.66 g, 11.2 mmol, 2.50 equiv) in 1,4-dioxane (30 mL) is heated at 100° C. After 1 h water is added, and the mixture is extracted with dichloromethane. The combined organic layers are concentrated under reduced pressure, and the residue is purified by flash column chromatography (silica gel, gradient cyclohexane/EtOAc 93:7 to cyclohexane/EtOAc 40:60) to yield the desired product.

| | |
|---|---|
| C₆H₉N₃O₂ | (MW = 155.1 g/mol) |
| ESI | 156 [M + H]⁺ |
| Rₜ (HPLC) | 0.30 min (X012_S01) |

### Intermediate 23

### 2-Bromo-5-((4-methoxybenzyl)oxy)pyrazine

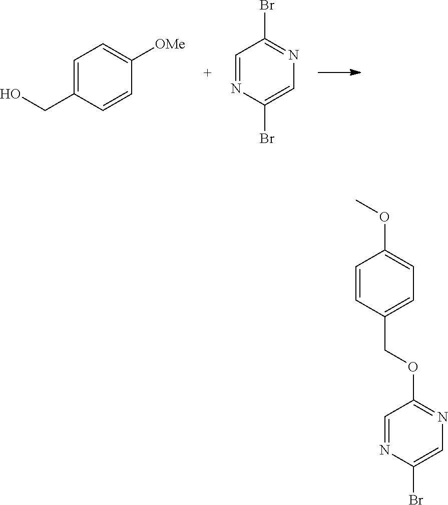 [SMILES: COc1ccc(CO)cc1 ; Brc1cnc(Br)cn1 ; COc1ccc(COc2cnc(Br)cn2)cc1]

Sodium hydride (60% mineral oil dispersion, 9.90 g, 248 mmol, 1.10 equiv) is added to a solution of 4-methoxybenzylalkohol (31.5 mL, 248 mmol, 1.10 equiv) in tetrahydrofuran (550 mL). After 30 min, 2,5-dibromopyrazine (54.6 g, 225 mmol, 1 equiv) is added. After 1.5 h water is added, and the precipitate is filtered and dried to yield the desired product.

| | |
|---|---|
| C₁₂H₁₁BrN₂O₂ | (MW = 294.0 g/mol) |
| ESI | 295 [M + H]⁺ |
| Rₜ (HPLC) | 0.67 min (X012_S01) |

### Intermediate 24

### 5-((4-Methoxybenzyl)oxy)pyrazin-2-amine

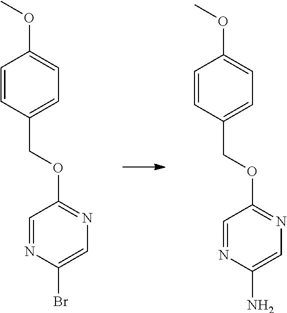 [SMILES: COc1ccc(COc2cnc(Br)cn2)cc1 ; COc1ccc(COc2cnc(N)cn2)cc1]

A solution of lithium-bis-(trimethylsilyl)-amide in tetrahydrofuran (1 M, 163 mL, 163 mmol, 1.20 equiv) is added dropwise to a mixture of 2-bromo-5-((4-methoxybenzyl)oxy)pyrazine (intermediate 23, 40.0 g, 136 mmol, 1 equiv) and XphosPdG3 (3.00 g, 3.50 mmol, 0.03 equiv) in toluene (450 mL) under argon atmosphere. After complete addition, the reaction mixture is heated at 110° C. After 3 h, the reaction mixture is allowed to cool to room temperature, water is added, and the mixture is filtered through a pad of Celite®. The layers are separated, and the organic layer is washed with water and concentrated under reduced pressure. The residue is dissolved in a mixture of methanol (200 mL) and acetic acid (0.5 mL) and stirred at room temperature. After 4 h, the mixture is filtered through a pad of Celite® and the filtrate is concentrated under reduced pressure. The residue is purified by flash column chromatography (silica gel, gradient cyclohexane/EtOAc 80:20 to cyclohexane/EtOAc 60:40) to yield the desired product.

| | |
|---|---|
| C₁₂H₁₃N₃O₂ | (MW = 231.1 g/mol) |
| ESI | 232 [M + H]⁺ |
| Rₜ (HPLC) | 0.49 min (X011_S05) |

### Intermediate 25

### 2-Isothiocyanato-5-((4-methoxybenzyl)oxy)pyrazine

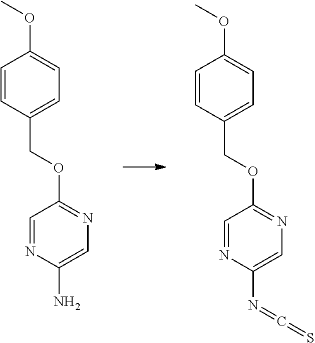 [SMILES: COc1ccc(COc2cnc(N)cn2)cc1 ; COc1ccc(COc2cnc(N=C=S)cn2)cc1]

1,1′-Thiocarbonylbis(pyridin-2(1H)-one) (14.8 g, 63.6 mmol, 1.10 equiv) is added to a solution of 5-((4-methoxybenzyl)oxy)pyrazin-2-amine (intermediate 24, 14.0 g, 60.5 mmol, 1 equiv) in acetonitrile (150 mL), and the reaction mixture is heated at 75° C. After 1 h, the reaction mixture is poured on water, and the mixture is extracted with ethyl acetate. The phases are separated, and the organic phase is dried over sodium sulfate. All volatiles are removed under reduced pressure, and the residue is purified by flash column chromatography (silica gel, gradient cyclohexane/EtOAc 95:5 to cyclohexane/EtOAc 70:30) to provide the product.

| | |
|---|---|
| C₁₃H₁₁N₃O₂S | (MW = 273.1 g/mol) |
| ESI | 274 [M + H]⁺ |
| Rₜ (HPLC) | 1.11 min (Z018_S04) |

### Intermediate 26

### Methyl 1-(3-(5-((4-methoxybenzyl)oxy)pyrazin-2-yl)thioureido)-4-nitro-1H-pyrazole-5-carboxylate

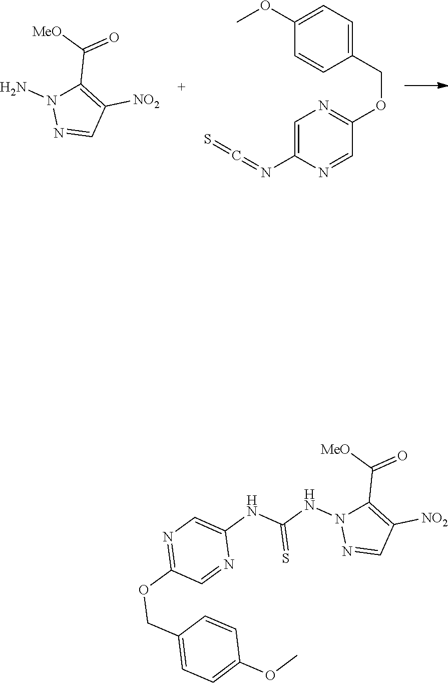 [SMILES: COC(=O)c1c([N+](=O)[O-])cnn1N ; COc1ccc(COc2cnc(N=C=S)cn2)cc1 ; COC(=O)c1c([N+](=O)[O-])cnn1NC(=S)Nc1cnc(OCc2ccc(OC)cc2)cn1]

2-Isothiocyanato-5-((4-methoxybenzyl)oxy)pyrazine (intermediate 25, 344 mg, 1.24 mmol, 1 equiv) is added to a solution of methyl 1-amino-4-nitro-1H-pyrazole-5-carboxylate (intermediate 12, 230 mg, 1.24 mmol, 1 equiv) in acetonitrile (2 mL) and the mixture is heated at 50° C. After 24 h, the reaction mixture is concentrated under reduced pressure to provide the product.

| | |
|---|---|
| C₁₈H₁₇N₇O₆S | (MW = 459.1 g/mol) |
| ESI | 460 [M + H]⁺ |
| Rₜ (HPLC) | 0.99 min (Z018_S04) |

### Intermediate 27

### 3-(5-((4-Methoxybenzyl)oxy)pyrazin-2-yl)-5-nitro-2-((2,4,6-trifluorobenzyl)thio)pyrazolo[5,1-f][1,2,4]triazin-4(3H)-one

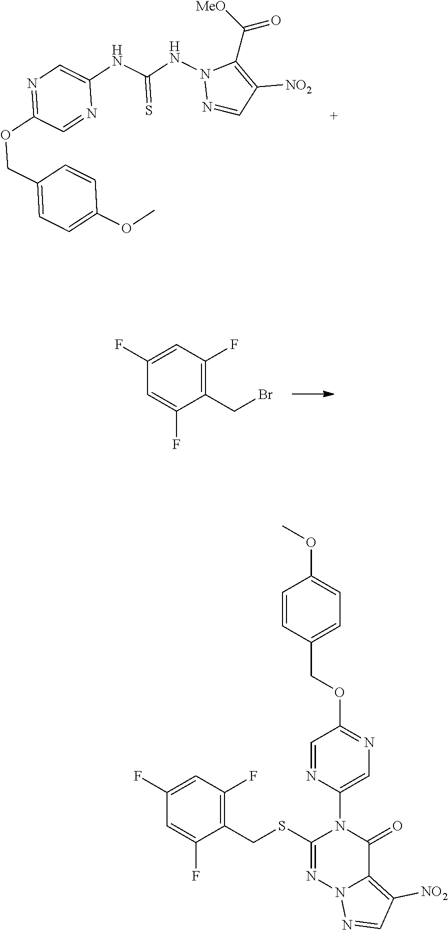 [SMILES: COC(=O)c1c([N+](=O)[O-])cnn1NC(=S)Nc1cnc(OCc2ccc(OC)cc2)cn1 ; Fc1cc(F)c(CBr)c(F)c1 ; COc1ccc(COc2cnc(-n3c(SCc4c(F)cc(F)cc4F)nn4ncc([N+](=O)[O-])c4c3=O)cn2)cc1]

Potassium tert-butoxide (147 mg, 1.31 mmol, 1 equiv) is added to a solution of methyl 1-(3-(5-((4-methoxybenzyl)oxy)pyrazin-2-yl)thioureido)-4-nitro-1H-pyrazole-5-carboxylate (intermediate 26, 600 mg, 1.31 mmol, 1 equiv) in acetonitrile (2 mL). After 16 h, 2-(bromomethyl)-1,3,5-trifluorobenzene (303 mg, 1.31 mmol, 1 equiv) is added. After 2 h, the reaction mixture is filtered and the filtrate is purified by reversed phase HPLC (Gilson Sunfire™-C18, gradient of acetonitrile in water, 0.1% TFA) to yield the desired product.

| | |
|---|---|
| C₂₄H₁₆F₃N₇O₅S | (MW = 571.1 g/mol) |
| ESI | 572 [M + H]⁺ |
| Rₜ (HPLC) | 1.14 min (Z018_S04) |

### Intermediate 28

### 5-(((Tert-butyldimethylsilyl)oxy)methyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

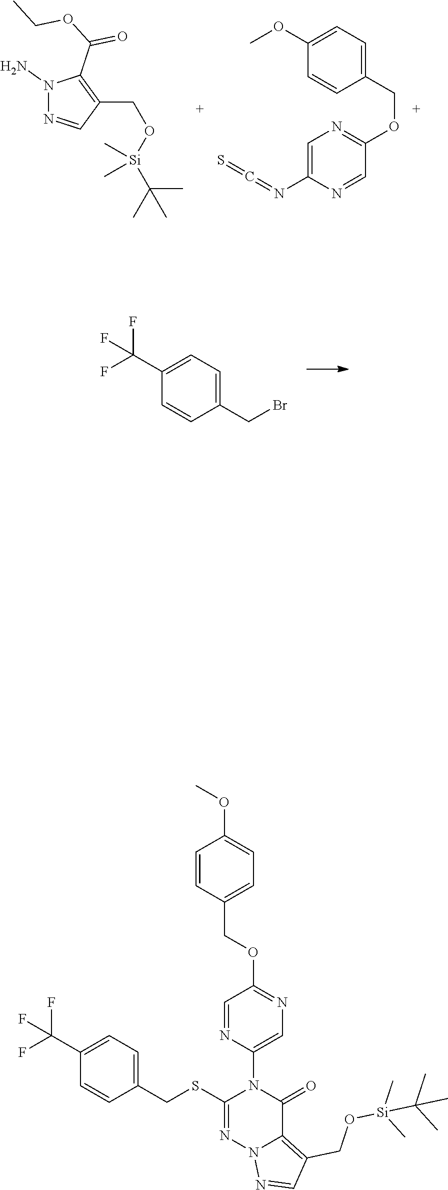 [SMILES: CCOC(=O)c1c(CO[Si](C)(C)C(C)(C)C)cnn1N ; COc1ccc(COc2cnc(N=C=S)cn2)cc1 ; FC(F)(F)c1ccc(CBr)cc1 ; COc1ccc(COc2cnc(-n3c(SCc4ccc(C(F)(F)F)cc4)nn4ncc(CO[Si](C)(C)C(C)(C)C)c4c3=O)cn2)cc1]

2-Isothiocyanato-5-((4-methoxybenzyl)oxy)pyrazine (intermediate 25, 2.90 g, 10.6 mmol, 1.10 equiv) is added to a solution of ethyl 1-amino-4-(((tert-butyldimethylsilyl)oxy)methyl)-1H-pyrazole-5-carboxylate (intermediate 10, 2.83 mg, 9.45 mmol, 1 equiv) and triethylamine (3.56 mL, 25.5 mmol, 2.70 equiv) in acetonitrile (40 mL) and the mixture is heated at 50° C. After 18 h, 1-(bromomethyl)-4-(trifluoromethyl)benzene (2.08 g, 8.51 mmol, 1 equiv) and triethylamine (2.63 mL, 18.9 mmol, 2.00 equiv) are added. After 2 h, water and dichloromethane are added, the layers are separated, and the organic layer is filtered through a phase transfer filter. All volatiles are removed under reduced pressure, and the residue is purified by flash column chromatography (silica gel, gradient cyclohexane to cyclohexane/EtOAc 70:30) to provide the product.

| | |
|---|---|
| C₃₂H₃₅F₃N₆O₄SSi | (MW = 684.2 g/mol) |
| ESI | 685 [M + H]⁺ |
| Rₜ (HPLC) | 0.97 min (X012_S01) |

The following compounds are prepared using procedures analogous to those described for intermediate 28, using appropriate starting materials. As is appreciated by those skilled in the art, the analogous examples may involve variations in general reaction conditions.

| | | | HPLC retention |
|---|---|---|---|
| | | | time [min] |
| Intermediate | Structure | ESI-MS | (method) |
| 29 | 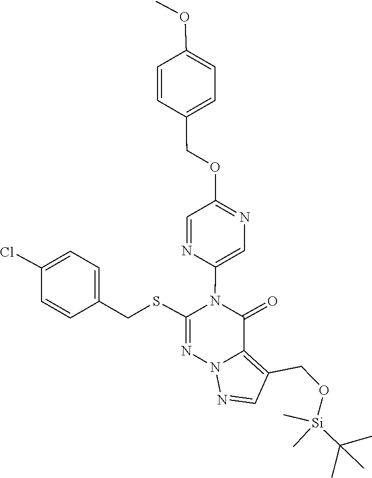 [SMILES: COc1ccc(COc2cnc(-n3c(SCc4ccc(Cl)cc4)nn4ncc(CO[Si](C)(C)C(C)(C)C)c4c3=O)cn2)cc1] | 651 [M + H]⁺ | 0.94 (X011_U04) |
| 30 | 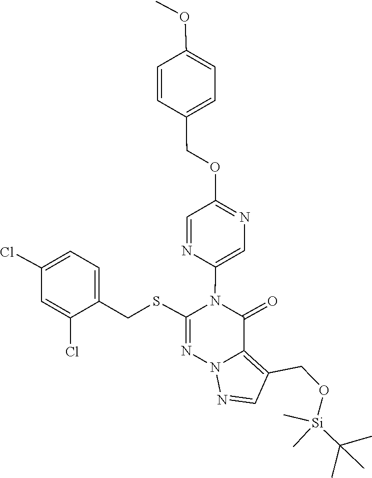 [SMILES: COc1ccc(COc2cnc(-n3c(SCc4ccc(Cl)cc4Cl)nn4ncc(CO[Si](C)(C)C(C)(C)C)c4c3=O)cn2)cc1] | 685 [M + H]⁺ | 0.98 (X011_U04) |
| 31 | 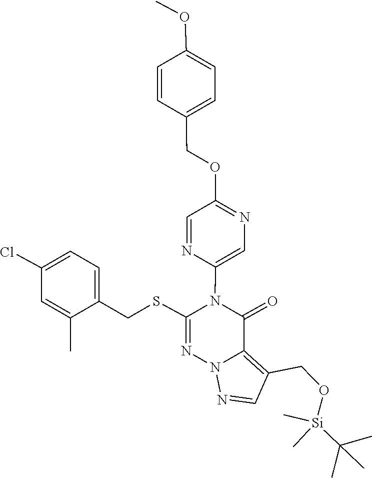 [SMILES: COc1ccc(COc2cnc(-n3c(SCc4ccc(Cl)cc4C)nn4ncc(CO[Si](C)(C)C(C)(C)C)c4c3=O)cn2)cc1] | 665 [M + H]⁺ | 0.96 (X011_U04) |
| 32 | 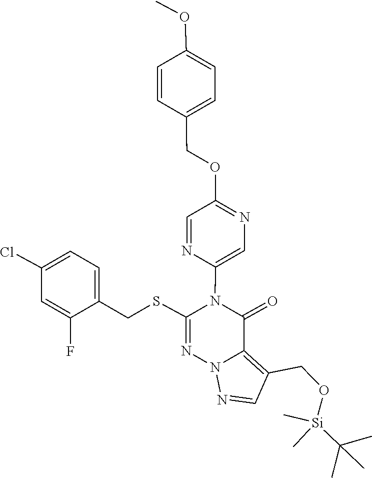 [SMILES: COc1ccc(COc2cnc(-n3c(SCc4ccc(Cl)cc4F)nn4ncc(CO[Si](C)(C)C(C)(C)C)c4c3=O)cn2)cc1] | 669 [M + H]⁺ | 0.94 (X011_U04) |

### Intermediate 3

### 5-(Hydroxymethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

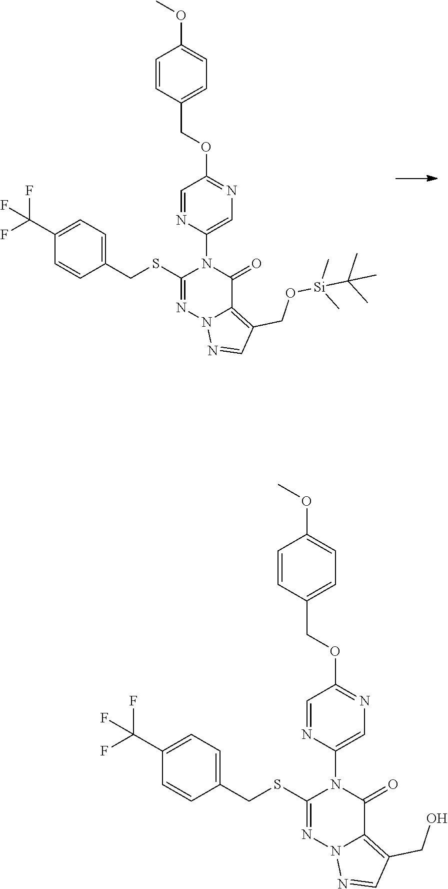 [SMILES: COc1ccc(COc2cnc(-n3c(SCc4ccc(C(F)(F)F)cc4)nn4ncc(CO[Si](C)(C)C(C)(C)C)c4c3=O)cn2)cc1 ; COc1ccc(COc2cnc(-n3c(SCc4ccc(C(F)(F)F)cc4)nn4ncc(CO)c4c3=O)cn2)cc1]

A solution of tetrabutylammonium fluoride (1 M in THF, 5.86 mL, 5.86 mmol, 1.22 equiv) is added to a solution of 5-(((tert-butyldimethylsilyl)oxy)methyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 28, 3.30 g, 4.82 mmol, 1 equiv) in tetrahydrofuran (41 mL). After 1 h, water and ethyl acetate are added, the layers are separated, and the aqueous layer is extracted with ethyl acetate. The combined organic layers are dried over magnesium sulfate, the dried solution is filtered, and the filtrate is concentrated under reduced pressure. The residue is purified by flash column chromatography (silica gel, gradient cyclohexane to cyclohexane/EtOAc 60:40) to provide the product.

| | |
|---|---|
| C₂₆H₂₁F₃N₆O₄S | (MW = 570.1 g/mol) |
| ESI | 571 [M + H]⁺ |
| Rₜ (HPLC) | 0.73 min (X012_S01) |

The following intermediates are prepared using procedures analogous to those described for intermediate 33, using appropriate starting materials. As is appreciated by those skilled in the art, the analogous examples may involve variations in general reaction conditions.

| | | | HPLC retention |
|---|---|---|---|
| | | | time [min] |
| Intermediate | Structure | ESI-MS | (method) |
| 34 | 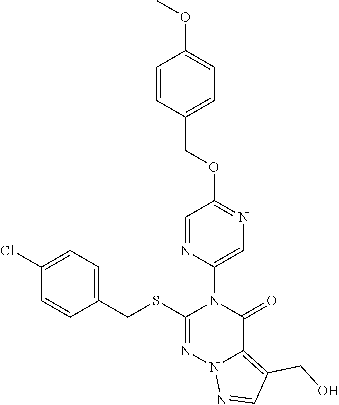 [SMILES: COc1ccc(COc2cnc(-n3c(SCc4ccc(Cl)cc4)nn4ncc(CO)c4c3=O)cn2)cc1] | 537 [M + H]⁺ | 0.73 (X012_S01) |
| 35 | 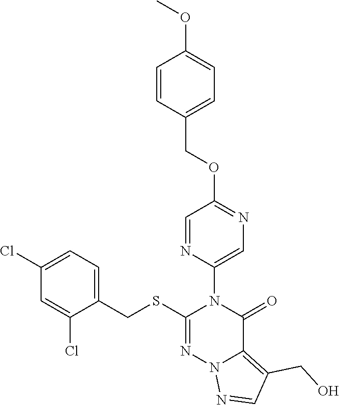 [SMILES: COc1ccc(COc2cnc(-n3c(SCc4ccc(Cl)cc4Cl)nn4ncc(CO)c4c3=O)cn2)cc1] | 571 [M + H]⁺ | 0.77 (X012_S01) |
| 36 | 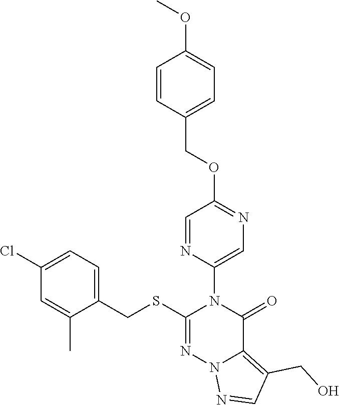 [SMILES: COc1ccc(COc2cnc(-n3c(SCc4ccc(Cl)cc4C)nn4ncc(CO)c4c3=O)cn2)cc1] | 551 [M + H]⁺ | 0.76 (X012_S01) |
| 37 | 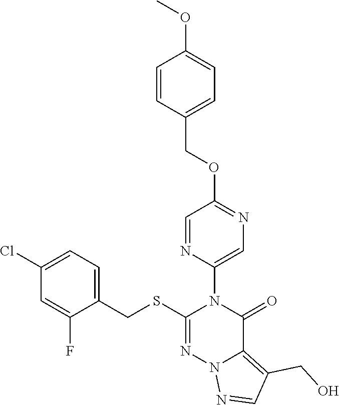 [SMILES: COc1ccc(COc2cnc(-n3c(SCc4ccc(Cl)cc4F)nn4ncc(CO)c4c3=O)cn2)cc1] | 555 [M + H]⁺ | 0.73 (X012_S01) |

### Intermediate 38

### 5-(Fluoromethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

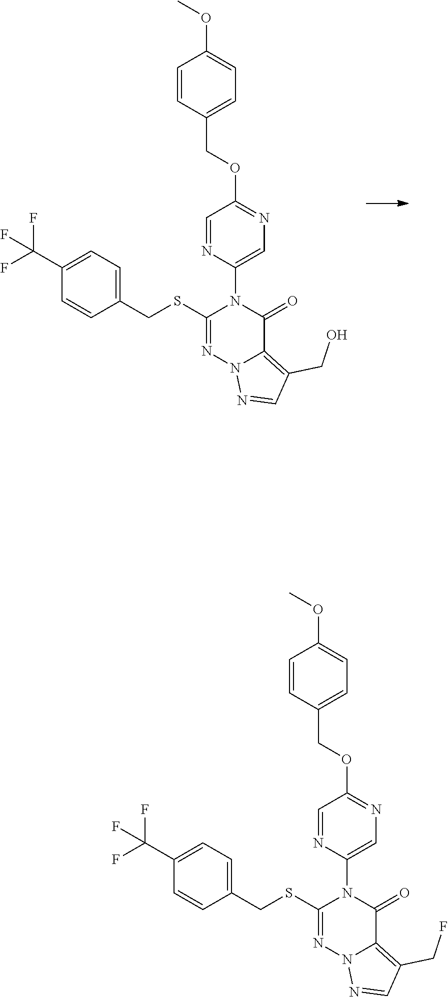 [SMILES: COc1ccc(COc2cnc(-n3c(SCc4ccc(C(F)(F)F)cc4)nn4ncc(CO)c4c3=O)cn2)cc1 ; COc1ccc(COc2cnc(-n3c(SCc4ccc(C(F)(F)F)cc4)nn4ncc(CF)c4c3=O)cn2)cc1]

A solution of bis(2-methoxyethyl)aminosulfur trifluoride (50% in THF, 39 μL, 0.11 mmol, 1.50 equiv) is added to solution of 5-(hydroxymethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 33, 50.0 mg, 70.0 μmol, 1 equiv) in dichloromethane (0.5 mL) at −78° C. After 1.5 h, additional bis(2-methoxyethyl)aminosulfur trifluoride (50% in THF, 13 μL, 0.35 mmol, 0.50 equiv) is added. After 2 h, the reaction mixture is poured into ice water and the mixture is extracted with dichloromethane and the organic layer is filtered through a phase transfer filter. All volatiles are removed under reduced pressure, and the residue is purified by flash column chromatography (silica gel, gradient cyclohexane/EtOAc 80:20 to cyclohexane/EtOAc 40:60) to provide the product.

| | |
|---|---|
| C₂₆H₂₀F₄N₆O₃S | (MW = 572.1 g/mol) |
| ESI | 573 [M + H]⁺ |
| Rₜ (HPLC) | 0.79 min (X012_S01) |

### Intermediate 39

### 3-(5-((4-Methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde

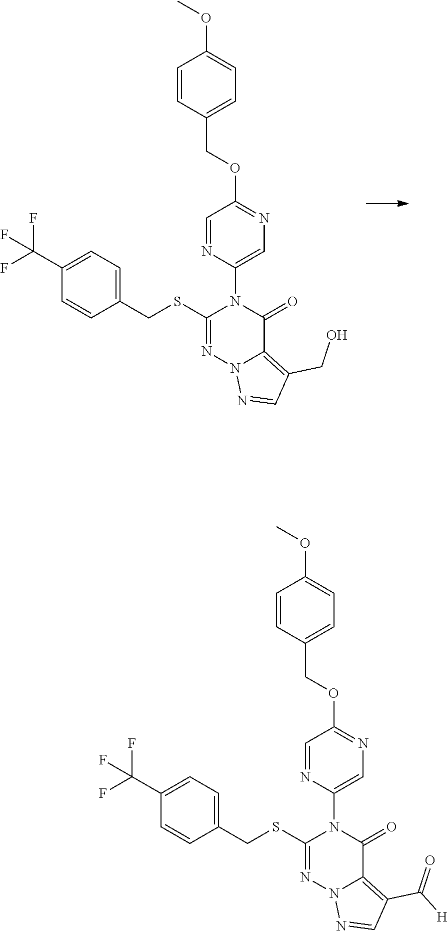 [SMILES: COc1ccc(COc2cnc(-n3c(SCc4ccc(C(F)(F)F)cc4)nn4ncc(CO)c4c3=O)cn2)cc1 ; COc1ccc(COc2cnc(-n3c(SCc4ccc(C(F)(F)F)cc4)nn4ncc(C=O)c4c3=O)cn2)cc1]

Dess-Martin periodinane (58.5 mg, 131 μmol, 1.20 equiv) is added to a solution of 5-(hydroxymethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 33, 65.0 mg, 114 μmol, 1 equiv) in dichloromethane (3 mL) at 0° C. and the reaction mixture is allowed to warm at room temperature. After 1 h, water is added, and the mixture is extracted with dichloromethane. The combined organic layers are filtered through a phase transfer filter, and the filtrate is concentrated under reduced pressure to yield the desired product.

| | |
|---|---|
| C₂₆H₁₉F₃N₆O₄S | (MW = 568.1 g/mol) |
| ESI | 569 [M + H]⁺ |
| Rₜ (HPLC) | 0.78 min (X012_S01) |

### Intermediate 40

### 3-(5-((4-Methoxybenzyl)oxy)pyrazin-2-yl)-4-oxo-2-((4-(trifluoromethyl)benzyl)thio)-3,4-dihydropyrazolo[5,1-f][1,2,4]triazine-5-carboxylic Acid

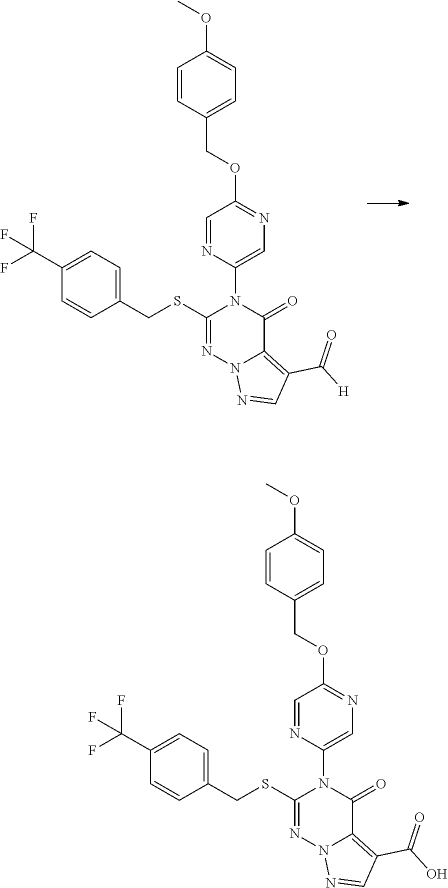 [SMILES: COc1ccc(COc2cnc(-n3c(SCc4ccc(C(F)(F)F)cc4)nn4ncc(C=O)c4c3=O)cn2)cc1 ; COc1ccc(COc2cnc(-n3c(SCc4ccc(C(F)(F)F)cc4)nn4ncc(C(=O)O)c4c3=O)cn2)cc1]

Sodium chlorite (40.0 mg, 354 μmol, 10.1 equiv) is added to a suspension of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 20.0 mg, 35.0 μmol, 1 equiv), phosphate buffer (pH=6.4, 290 μL) and tert-butanol (400 μL, 4.32 mmol, 123 equiv). After 16 h, THF (1 mL) is added. After 16 h, the precipitate is filtered and dried to yield the desired product.

| | |
|---|---|
| C₂₆H₁₉F₃N₆O₅S | (MW = 584.1 g/mol) |
| ESI | 585 [M + H]⁺ |
| Rₜ (HPLC) | 0.76 min (X012_S01) |

### Intermediate 41

### 5-(1-Hydroxyethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

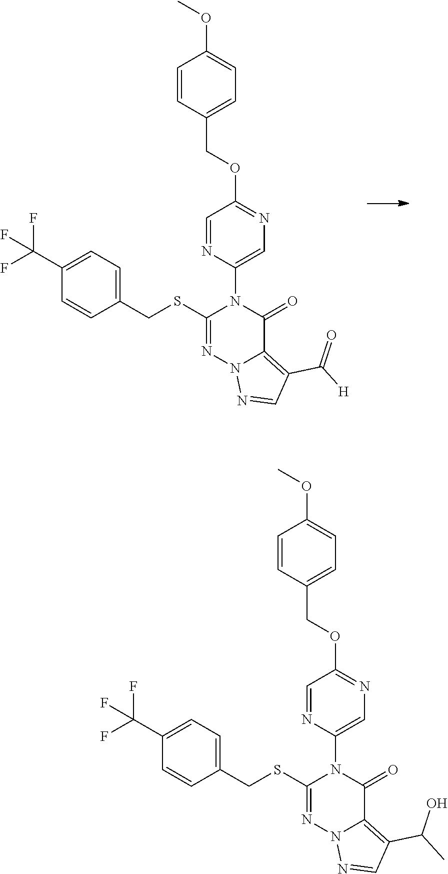 [SMILES: COc1ccc(COc2cnc(-n3c(SCc4ccc(C(F)(F)F)cc4)nn4ncc(C=O)c4c3=O)cn2)cc1 ; COc1ccc(COc2cnc(-n3c(SCc4ccc(C(F)(F)F)cc4)nn4ncc(C(C)O)c4c3=O)cn2)cc1]

A solution of methyl magnesium bromide (3 M in THF, 30.0 μL, 90.0 μmol, 0.500 equiv) is added to a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 100 mg, 172 μmol, 1 equiv) in tetrahydrofuran (2 mL) at −78° C. After 30 min, water is added, and the mixture is extracted with ethylacetate. The combined organic layers are dried over magnesium sulfate, the dried solution is filtered, and the filtrate is concentrated. The residue is purified by reversed phase HPLC (XBridge™-C18, gradient of acetonitrile in water, 0.1% TFA) to yield the desired product.

| | |
|---|---|
| C₂₇H₂₁F₃N₆O₄S | (MW = 584.1 g/mol) |
| ESI | 585 [M + H]⁺ |
| Rₜ (HPLC) | 0.76 min (X012_S01) |

### Intermediate 42

### 3-(5-((4-Methoxybenzyl)oxy)pyrazin-2-yl)-4-oxo-2-((4-(trifluoromethyl)benzyl)thio)-3,4-dihydropyrazolo[5,1-f][1,2,4]triazine-5-carboxamide

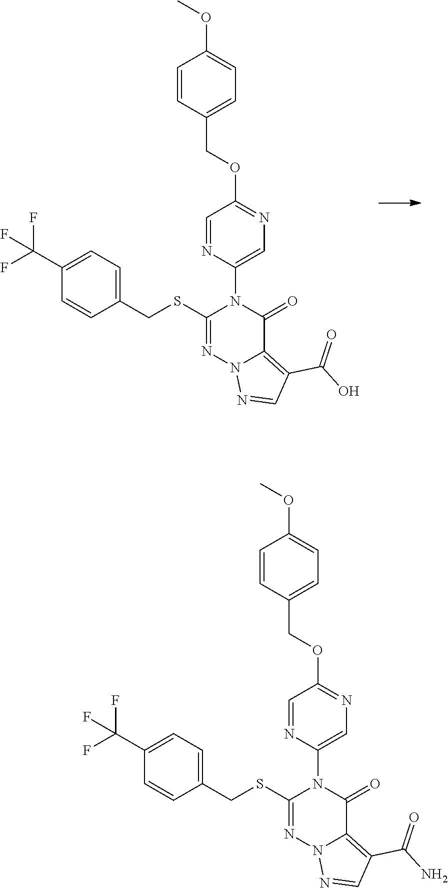 [SMILES: COc1ccc(COc2cnc(-n3c(SCc4ccc(C(F)(F)F)cc4)nn4ncc(C(=O)O)c4c3=O)cn2)cc1 ; COc1ccc(COc2cnc(-n3c(SCc4ccc(C(F)(F)F)cc4)nn4ncc(C(N)=O)c4c3=O)cn2)cc1]

Ammonium hydrochloride (10 mg, 187 μmol, 5.20 equiv) is added to a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carboxylic acid (intermediate 40, 25.0 mg, 36.0 μmol, 1 equiv), triethylamine (60.0 μL, 433 μmol, 12.0 equiv) and 1-propanephosphonic anhydride (50% in THF, 80 μL, 134 μmol, 3.70 equiv) in tetrahydrofuran (0.5 mL). After 48 h, the mixture is filtered, the filtrate is purified by reversed phase HPLC (Sunfire™-C18, gradient of acetonitrile in water, 0.1% TFA) to yield the desired product.

| | |
|---|---|
| C₂₆H₂₀F₃N₇O₄S | (MW = 583.1 g/mol) |
| ESI | 584 [M + H]⁺ |
| Rₜ (HPLC) | 1.13 min (X018_S03) |

### Intermediate 43

### 3-(5-((4-Methoxybenzyl)oxy)pyrazin-2-yl)-N-methyl-4-oxo-2-((4-(trifluoromethyl)benzyl)thio)-3,4-dihydropyrazolo[5,1-f][1,2,4]triazine-5-carboxamide

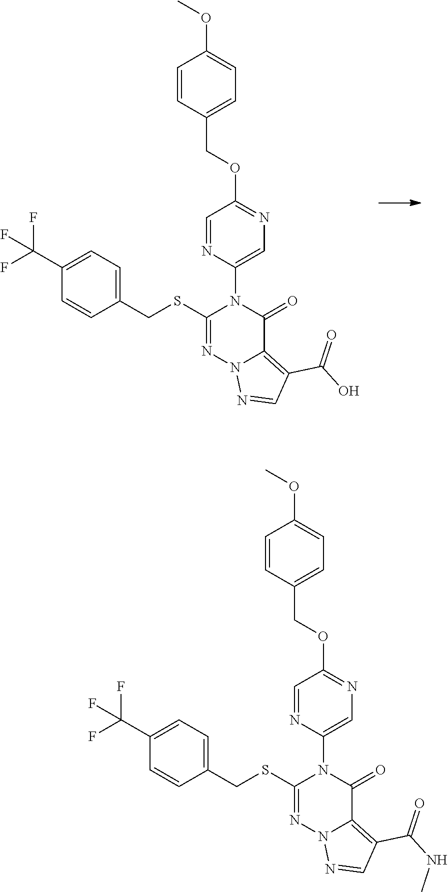 [SMILES: COc1ccc(COc2cnc(-n3c(SCc4ccc(C(F)(F)F)cc4)nn4ncc(C(=O)O)c4c3=O)cn2)cc1 ; CNC(=O)c1cnn2nc(SCc3ccc(C(F)(F)F)cc3)n(-c3cnc(OCc4ccc(OC)cc4)cn3)c(=O)c12]

Intermediate 43 is prepared using procedures analogous to those described for intermediate 42, using appropriate starting materials.

| | |
|---|---|
| C₂₇H₂₂F₃N₇O₄S | (MW = 597.1 g/mol) |
| ESI | 598 [M + H]⁺ |
| Rₜ (HPLC) | 1.08 min (X018_S03) |

### Intermediate 44

### 3-(5-((4-Methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde O-Methyl Oxime

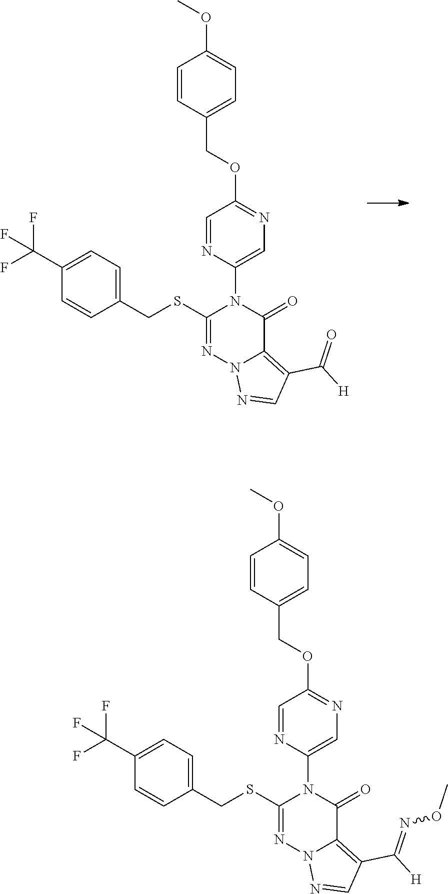 [SMILES: COc1ccc(COc2cnc(-n3c(SCc4ccc(C(F)(F)F)cc4)nn4ncc(C=O)c4c3=O)cn2)cc1 ; CON=Cc1cnn2nc(SCc3ccc(C(F)(F)F)cc3)n(-c3cnc(OCc4ccc(OC)cc4)cn3)c(=O)c12]

O-Methylhydroxylamine hydrochloride (18.0 mg, 210 μmol, 1 equiv) is added to a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 117 mg, 206 μmol, 1 equiv) and pyridine (17.0 μL, 211 μmol, 1 equiv) in dichloromethane. After 16 h, water is added, and the mixture is extracted with dichloromethane. The combined organic layers are filtered through a phase transfer filter. All volatiles are removed under reduced to provide the product.

| | |
|---|---|
| C₂₇H₂₂F₃N₇O₄S | (MW = 597.1 g/mol) |
| ESI | 598 [M + H]⁺ |
| Rₜ (HPLC) | 0.83 min (X012_S01) |

### Intermediate 45

### 3-(5-((4-Methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde Oxime

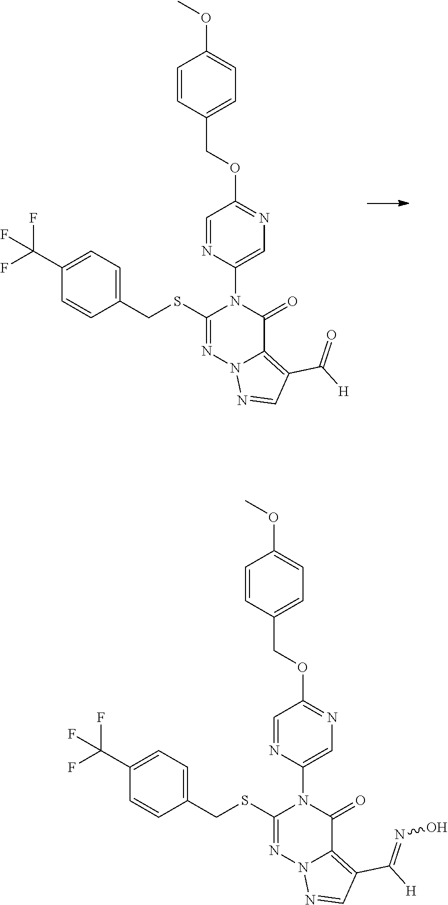 [SMILES: COc1ccc(COc2cnc(-n3c(SCc4ccc(C(F)(F)F)cc4)nn4ncc(C=O)c4c3=O)cn2)cc1 ; COc1ccc(COc2cnc(-n3c(SCc4ccc(C(F)(F)F)cc4)nn4ncc(C=NO)c4c3=O)cn2)cc1]

Hydroxylamine hydrochloride (10 mg, 141 μmol, 1 equiv) is added to a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 75 mg, 132 μmol, 1 equiv) and pyridine (11.0 μL, 136 μmol, 1 equiv) in dichloromethane (1.0 mL), and the mixture is stirred at room temperature over night. Another portion of hydroxylamine hydrochloride (10 mg, 141 μmol, 1 equiv) and pyridine (11.0 μL, 136 μmol, 1 equiv) is added, and the mixture is stirred over night. Water is added, and the mixture is extracted with dichloromethane. The combined organic layers are dried using a Biotage ISOLUTE® phase separator and concentrated under reduced pressure to give the desired product.

| | |
|---|---|
| C₂₆H₂₀F₃N₇O₄S | (MW = 583.1 g/mol) |
| ESI | 584 [M + H]⁺ |
| Rₜ (HPLC) | 0.76 min (X012_S01) |

### Intermediate 46

### 3-(5-((4-Methoxybenzyl)oxy)pyrazin-2-yl)-2-((4-(trifluoromethyl)benzyl)thio)-5-vinylpyrazolo[5,1-f][1,2,4]triazin-4(3H)-one

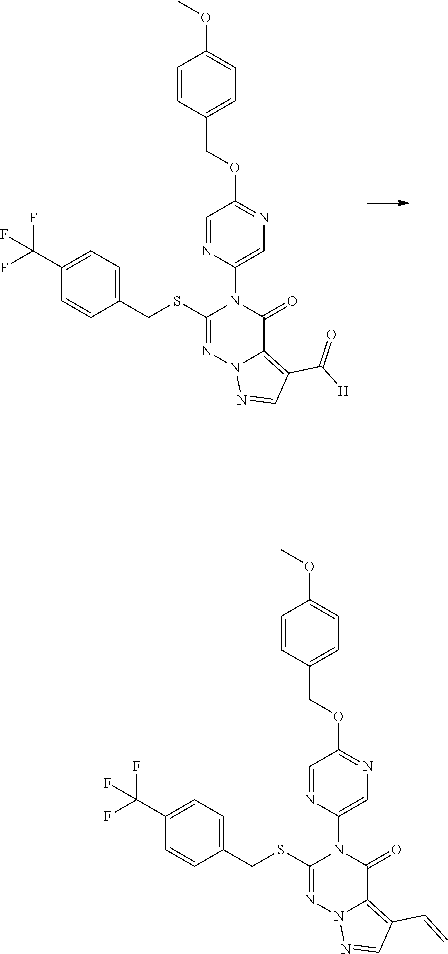 [SMILES: COc1ccc(COc2cnc(-n3c(SCc4ccc(C(F)(F)F)cc4)nn4ncc(C=O)c4c3=O)cn2)cc1 ; C=Cc1cnn2nc(SCc3ccc(C(F)(F)F)cc3)n(-c3cnc(OCc4ccc(OC)cc4)cn3)c(=O)c12]

A solution on n-butyl lithium in n-hexane (2.5M, 169 μL, 423 μmol, 1.20 equiv) is added to a suspension of methyltriphenylphosphonium bromide (154 mg, 422 μmol, 1.20 equiv) in tetrahydrofuran (4 mL) at −78° C. After addition, the reaction mixture is allowed to warm to 0° C. After 0.5 h, the reaction mixture is cooled to −78° C. and a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 200 mg, 352 μmol, 1 equiv) in tetrahydrofuran (4 mL) is added. After complete addition, the reaction mixture is allowed to warm to room temperature. After 60 min, water and ethyl acetate are added, the layers are separated, and the aqueous layer is extracted with ethyl acetate. The combined organic layers are dried over magnesium sulfate, the dried solution is filtered, and the filtrate is concentrated under reduced pressure. The residue is purified by flash column chromatography (silica gel, gradient cyclohexane/EtOAc 93:7 to cyclohexane/EtOAc 60:40) to yield the desired product.

| | |
|---|---|
| C₂₇H₂₁F₃N₆O₃S | (MW = 566.1 g/mol) |
| ESI | 567 [M + H]⁺ |
| Rₜ (HPLC) | 0.84 min (X012_S01) |

### Intermediate 47

### 5-(2-Fluorovinyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

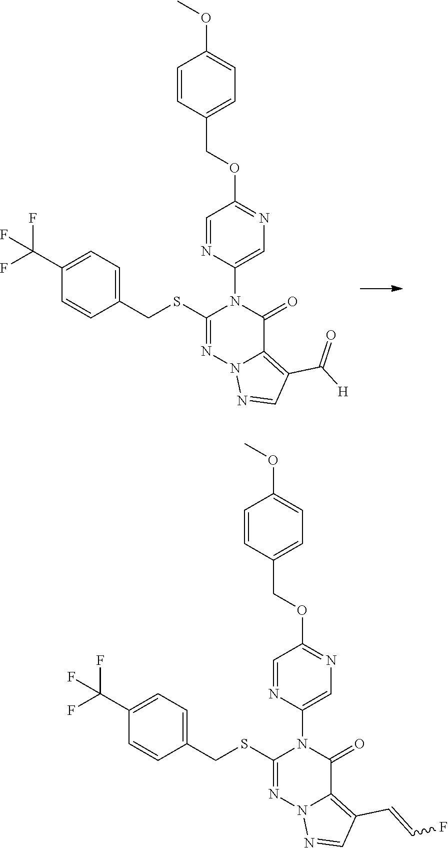

Under an atmosphere of argon, potassium tert-butoxide (30.0 mg, 267 μmol, 1.0 equiv) is added to a mixture of (fluoromethyl)triphenylphosphonium tetrafluoroborate (114.0 mg, 293 μmol, 1.1 equiv) in tetrahydrofuran (3.8 mL) at −78° C. (dry ice bath). The mixture is stirred for 10 min, and 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 150.0 mg, 264 μmol, 1.0 equiv) is added. The mixture is allowed to slowly warm to room temperature, and stirred over night. In a separate flask, a mixture of (fluoromethyl)triphenylphosphonium tetrafluoroborate (114.0 mg, 293 μmol, 1.1 equiv) in tetrahydrofuran (3.8 mL) is cooled at −78° C. (dry ice bath), treated with potassium tert-butoxide (30.0 mg, 267 μmol, 1.0 equiv), and stirred at −78° C. for 15 min before being added to the reaction mixture. The mixture is warmed at room temperature, stirred for 3 h, and diluted with water and extracted with ethyl acetate. The combined organic layers are dried over MgSO₄ and concentrated under reduced pressure. The residue is purified by reversed phase HPLC (XBridge™-C18, gradient of acetonitrile in water, 0.1% TFA) to yield the desired product as a 8:2 mixture of (E)- and (Z)-isomers.

| | |
|---|---|
| C₂₇H₂₀F₄N₆O₃S | (MW = 584.1 g/mol) |
| ESI | 585 [M + H]⁺ |
| Rₜ (HPLC) | 0.83 min (X012_S01) |

### Intermediate 48

### 5-(2,2-Difluorovinyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one

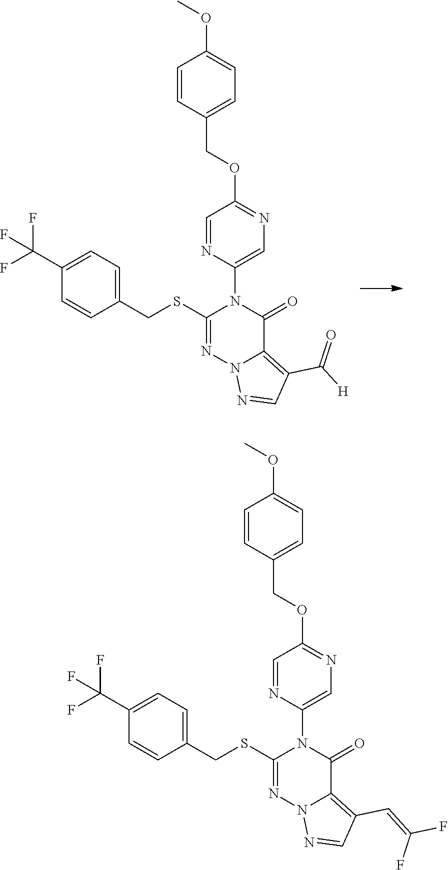

2,2-Difluoro-2-(triphenylphosphonio)acetate (100 mg, 281 μmol, 1.60 equiv) is added to a solution of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 39, 100 mg, 176 μmol, 1 equiv) in acetonitrile (2.0 mL) at 60° C. The mixture is stirred at 60° C. for 30 min, diluted with water (1.0 mL) and acetonitrile (2.0 mL). The mixture is filtered and purified by reversed phase HPLC (XBridge™-C18, gradient of acetonitrile in water, 0.1% NH to yield the desired product.

| | |
|---|---|
| C₂₇H₁₉F₅N₆O₃S | (MW = 602.1 g/mol) |
| ESI | 603 [M + H]⁺ |
| Rₜ (HPLC) | 0.86 min (X018_S03) |

### Intermediate 49

### 5-Fluoro-2-((4-iodobenzyl)thio)-3-(5-((4-methoxybenzyl)oxy)pyrazin-2-yl)pyrazolo[5,1-f][1,2,4]triazin-4(3H)-one

### Step 1

### 5-Fluoro-3-(5-((4-methoxybenzyl)oxy)pyrazin-2-yl)-2-thioxo-2,3-dihydropyrazolo[5,1-f][1,2,4]triazin-4(1H)-one

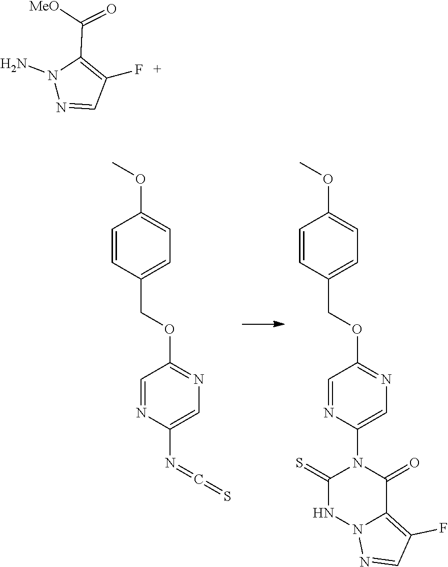

A mixture of methyl 1-amino-4-fluoro-1H-pyrazole-5-carboxylate (intermediate 1, 1.00 g, 6.29 mmol, 1.0 equiv) and 2-isothiocyanato-5-((4-methoxybenzyl)oxy)pyrazine (intermediate 25, 1.75 g, 6.29 mmol, 1.0 equiv) in acetonitrile (20 mL) is stirred for 72 h. Potassium tert-butoxide (705 mg, 6.29 mmol, 1.0 equiv) is added, and the mixture is stirred for 4 h. An aliquot (approximately ⅔≈67%) of the mixture is used in the next step without further purification.

### Step 2

### 5-Fluoro-2-((4-iodobenzyl)thio)-3-(5-((4-methoxybenzyl)oxy)pyrazin-2-yl)pyrazolo[5,1-f][1,2,4]triazin-4(3H)-one

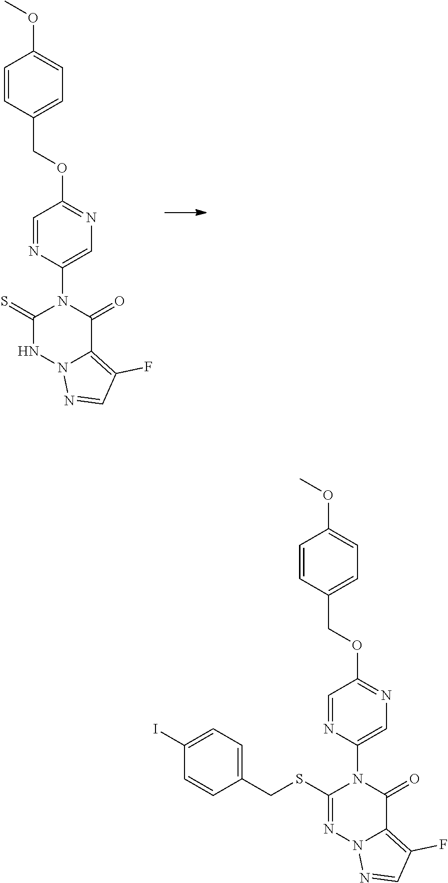 [SMILES: COc1ccc(COc2cnc(-n3c(=S)[nH]n4ncc(F)c4c3=O)cn2)cc1 ; COc1ccc(COc2cnc(-n3c(SCc4ccc(I)cc4)nn4ncc(F)c4c3=O)cn2)cc1]

An aliquot (approximately ⅔≈67%) of the mixture from step 1 is treated with 4-iodobenzyl bromide (1.28 g, 4.17 mmol, 1 equiv). The mixture is stirred at room temperature for 2 h. Water is added, and the mixture is extracted with ethyl acetate. The organic layer is washed with water and concentrated under reduced pressure. The residue is purified by reversed phase HPLC (Gilson Sunfire™-C18, gradient of acetonitrile in water, 0.1% TFA) to yield the desired product.

| | |
|---|---|
| C₂₄H₁₈FN₆O₃S | (MW = 616.0 g/mol) |
| ESI | 617 [M + H]⁺ |
| Rₜ (HPLC) | 1.20 min (Z018_S04) |

### Intermediate 50

### 4-(((5-Fluoro-3-(5-((4-methoxybenzyl)oxy)pyrazin-2-yl)-4-oxo-3,4-dihydropyrazolo[5,1-f][1,2,4]triazin-2-yl)thio)methyl)benzaldehyde

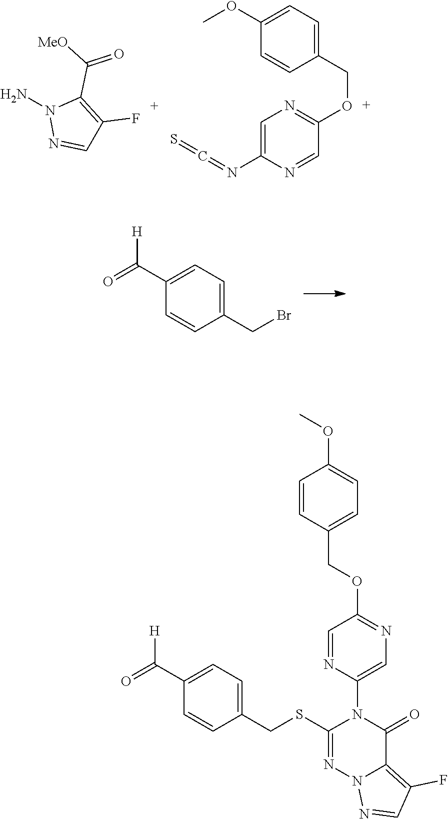

2-Isothiocyanato-5-((4-methoxybenzyl)oxy)pyrazine (intermediate 25, 526 mg, 1.89 mmol, 1 equiv) is added to a solution of methyl 1-amino-4-fluoro-1H-pyrazole-5-carboxylate (intermediate 1, 300 mg, 1.89 mmol, 1 equiv) and potassium tert-butoxide (212 mg, 1.89 μmol, 1 equiv) in acetonitrile (6 mL). After 72 h, 4-(bromomethyl)benzaldehyde (387 mg, 1.89 mmol, 1 equiv) is added. After 2 h, water is added and the mixture is extracted with ethyl acetate. The combined organic layers are concentrated under reduced pressure and the residue is purified by reversed phase HPLC (Gilson Sunfire™-C18, gradient of acetonitrile in water, 0.1% TFA) to yield the desired product.

| | |
|---|---|
| C₂₅H₁₉FN₆O₄S | (MW = 518.1 g/mol) |
| ESI | 519 [M + H]⁺ |
| Rₜ (HPLC) | 1.08 min (Z018_S04) |

### Intermediate 51

### 5-Fluoro-2-((4-(2-fluorovinyl)benzyl)thio)-3-(5-((4-methoxybenzyl)oxy)pyrazin-2-yl)pyrazolo[5,1-f][1,2,4]triazin-4(3H)-one

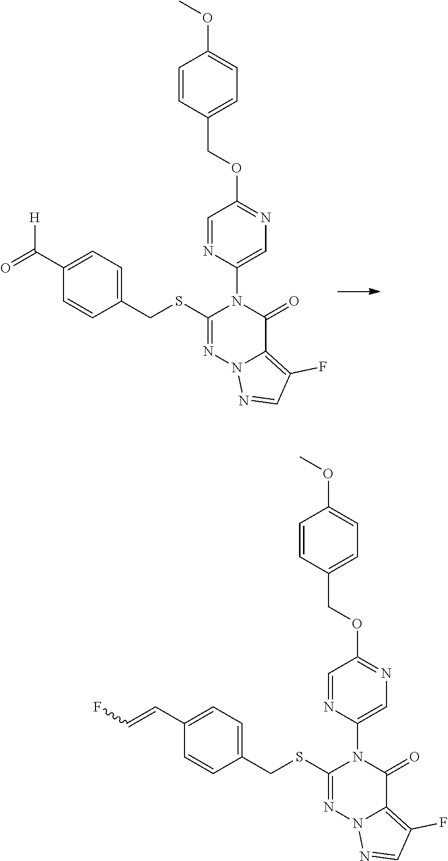 [SMILES: COc1ccc(COc2cnc(-n3c(SCc4ccc(C=O)cc4)nn4ncc(F)c4c3=O)cn2)cc1 ; COc1ccc(COc2cnc(-n3c(SCc4ccc(C=CF)cc4)nn4ncc(F)c4c3=O)cn2)cc1]

Potassium tert-butoxide (39.0 mg, 347 μmol, 0.900 equiv) is added to a solution of (fluoromethyl)triphenylphosphonium tetrafluoroborate (150 mg, 386 μmol, 1 equiv) in tetrahydrofuran (5 mL) at 0° C. After 15 min, 4-(((5-fluoro-3-(5-((4-methoxybenzyl)oxy)pyrazin-2-yl)-4-oxo-3,4-dihydropyrazolo[5,1-f][1,2,4]triazin-2-yl)thio)methyl)benzaldehyde (intermediate 50, 200 mg, 386 μmol, 1 equiv) is added and the reaction mixture is allowed to warm at room temperature. After 16 h, potassium tert-butoxide (39.0 mg, 347 μmol, 0.900 equiv) and (fluoromethyl)triphenylphosphonium tetrafluoroborate (150 mg, 386 μmol, 1 equiv) are added and the reaction mixture is heated at 70° C. After 18 h, water and ethyl acetate are added, the layers are separated, and the organic phase is washed with water. The organic layer is concentrated under reduced pressure and the residue is purified by reversed phase HPLC (Gilson Sunfire™-C18, gradient of acetonitrile in water, 0.1% TFA) to yield the desired product as a 1:1 mixture of (E)- and (Z)-isomers.

| | |
|---|---|
| C₂₆H₂₀F₂N₆O₃S | (MW = 534.1 g/mol) |
| ESI | 535 |
| Rₜ (HPLC) | 1.15 min (Z018_S04) |

### Intermediate 52

### 6-Fluoro-5-methoxypyrazin-2-amine

### Step 1

### 5-Bromo-3-fluoro-2-methoxypyrazine

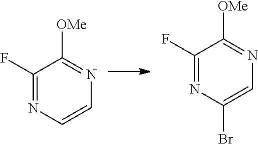 [SMILES: COc1nccnc1F ; COc1ncc(Br)nc1F]

3-Nitrobenzenesulfonic acid (150 mg, 724 μmol, 0.100 equiv) and 1,3-dibromo-5,5-dimethylhydantoin (1.30 g, 4.41 mmol, 0.600 equiv) are added to a solution of 2-fluoro-3-methoxypyrazine (1.00 g, 7.42 mmol, 1 equiv) in 1,1,1,3,3,3-hexafluoropropan-2-ol (5 mL), and the reaction mixture is heated at 60° C. After 18 h, the reaction mixture is concentrated under reduced pressure (50° C., 84 mbar). The residue is dissolved in dichloromethane, and the organic phase is washed with water. The organic layer is concentrated under reduced pressure (50° C., 84 mbar), and the residue is purified by flash column chromatography (silica gel, gradient cyclohexane to cyclohexane/EtOAc 90:10) to yield the desired product.

| | |
|---|---|
| C₅H₄BrFN₂O | (MW = 205.9 g/mol) |
| ESI | not detected |
| Rₜ (HPLC) | 0.93 min (Z018_S04) |

### Step 2

### 6-Fluoro-5-methoxypyrazin-2-amine

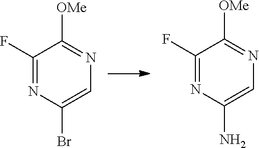 [SMILES: COc1ncc(Br)nc1F ; COc1ncc(N)nc1F]

Under an atmosphere of argon, a solution of lithium bis(trimethylsilyl)amide in tetrahydrofuran (1 M, 4.35 mL, 4.35 mmol, 3.00 equiv) is added dropwise to a mixture of 5-bromo-3-fluoro-2-methoxypyrazine (step 1, 300 mg, 1.45 mmol, 1 equiv) and bis(di-tert-butyl(4-dimethylaminophenyl)phosphine)dichloropalladium(II) (67.0 mg, 145 μmol, 0.100 equiv) in 1,4-dioxane (6 mL). After complete addition, the reaction mixture is heated at 110° C. After 18 h, the reaction mixture cooled at room temperature and concentrated under reduced pressure (50° C., 180 mbar). The residue is purified by flash column chromatography (silica gel, gradient cyclohexane to cyclohexane/EtOAc 50:50) to yield the desired product.

| | |
|---|---|
| C₅H₆FN₃O | (MW = 143.0 g/mol) |
| ESI | 144 [M + H]⁺ |
| Rₜ (HPLC) | 0.58 min (Z018_S04) |

### Intermediate 53

### 5-Fluoro-3-(6-fluoro-5-methoxypyrazin-2-yl)-2-((4-(trifluoromethyl)benzyl)thio)pyrazolo[5,1-f][1,2,4]triazin-4(3H)-one

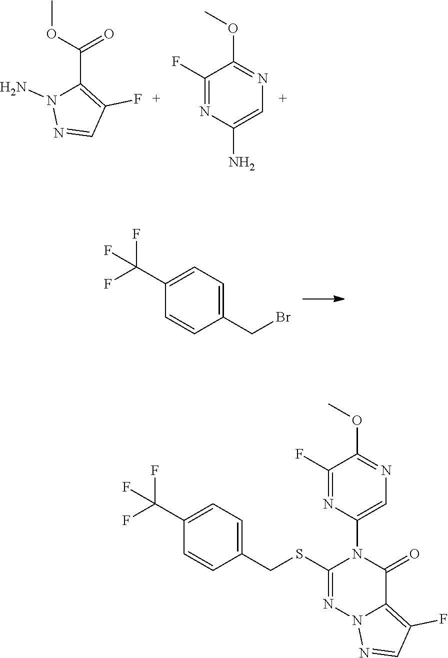

6-Fluoro-5-methoxypyrazin-2-amine (intermediate 52, 184 mg, 992 μmol, 1 equiv) is added to a mixture of 1,1′-thiocarbonylbis(pyridin-2(1H)-one) (270 mg, 1.16 mmol, 1.20 equiv) in acetonitrile (5 mL) and the mixture is heated at 75° C. After 3 h, the reaction mixture is cooled to room temperature and methyl 1-amino-1H-pyrazole-5-carboxylate (intermediate 1, 150 mg, 943 μmol, 1 equiv) is added. After 1.5 h, potassium tert-butoxide (117 mg, 1.04 mmol, 1.10 equiv). After 1.5 h, 1-(bromomethyl)-4-(trifluoromethyl)benzene (237 mg, 972 μmol, 1 equiv) is added. After 45 min, the reaction mixture is concentrated and the residue is purified by reversed phase HPLC (Agilent Sunfire™-C18, gradient of acetonitrile in water, 0.1% TFA) to yield the desired product.

| | |
|---|---|
| C₁₈H₁₁F₅N₆O₂S | (MW = 470.1 g/mol) |
| ESI | 471 [M + H]⁺ |
| Rₜ (HPLC) | 1.13 min (Z018_S04) |

### Intermediate 54

### Tert-butyl (3-fluoro-5-methoxypyrazin-2-yl)carbamate

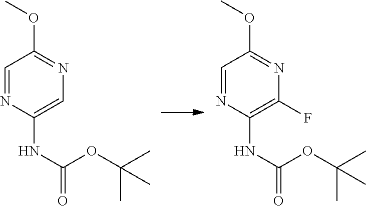 [SMILES: COc1cnc(NC(=O)OC(C)(C)C)cn1 ; COc1cnc(NC(=O)OC(C)(C)C)c(F)n1]

1-(Chloromethyl)-4-fluoro-1,4-diazabicyclo[2.2.2]octane-1,4-diium ditetrafluoroborate (Selectfluor) (2.25 g, 6.35 mmol, 3.78 equiv) is added to a solution of tert-butyl (5-methoxypyrazin-2-yl)carbamate (379 mg, 1.68 mmol, 1 equiv) in acetonitrile (6 mL). After 48 h, the reaction mixture is filtered, and the filtrate is concentrated under reduced pressure. The residue is purified by flash column chromatography (silica gel, gradient cyclohexane to cyclohexane/EtOAc 4:1) to yield the desired product.

| | |
|---|---|
| C₁₀H₁₄FN₃O₃ | (MW = 243.1 g/mol) |
| ESI | 188 [M + H-t-Bu]⁺ |
| Rₜ (HPLC) | 0.88 min (Z018_S04) |

### Intermediate 55

### 3-Fluoro-5-methoxypyrazin-2-amine

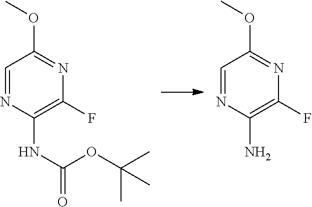

Trifluoroacetic acid (2 mL) is added to a solution of tert-butyl (3-fluoro-5-methoxypyrazin-2-yl)carbamate (intermediate 54, 70.0 mg, 311 μmol, 1 equiv) in dichloromethane (2 mL). After 1 h, the reaction mixture is concentrated under reduced pressure to yield the desired product.

| | |
|---|---|
| C₅H₆FN₃O | (MW = 143.0 g/mol) |
| ESI | 144 [M + H]⁺ |
| Rₜ (HPLC) | 0.55 min (Z018_S04) |

### Intermediate 56

### 5-Fluoro-3-(6-fluoro-5-methoxypyrazin-2-yl)-2-((4-(trifluoromethyl)benzyl)thio)pyrazolo[5,1-f][1,2,4]triazin-4(3H)-one

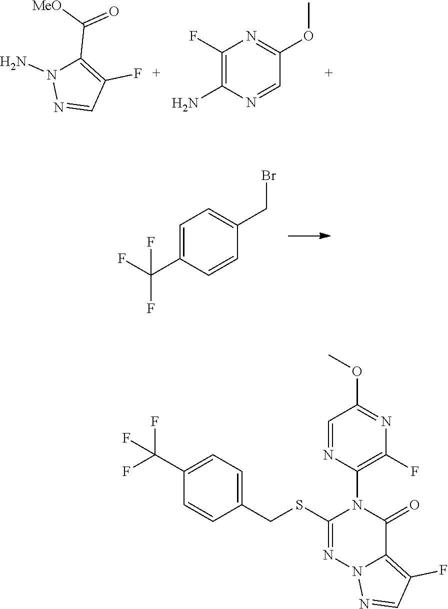

Intermediate 56 is prepared using procedures analogous to those described for intermediate 53, using appropriate starting materials.

| | |
|---|---|
| C₁₈H₁₁F₅N₆O₂S | (MW = 470.1 g/mol) |
| ESI | 471 [M + H]⁺ |
| Rₜ (HPLC) | 1.12 min (Z018_S04) |

### Intermediate 57

### Methyl 1-(3-(5-((4-methoxybenzyl)oxy)pyrazin-2-yl)thioureido)-1H-pyrazole-5-carboxylate

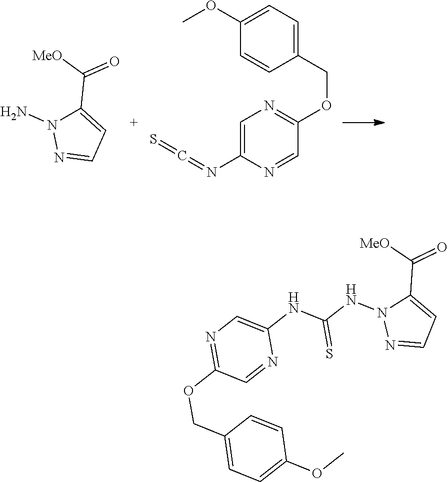

Intermediate 57 is prepared using procedures analogous to those described for intermediate 26, using appropriate starting materials.

| | |
|---|---|
| C₁₈H₁₈N₆O₄S | (MW = 414.1 g/mol) |
| ESI | 415 [M + H]⁺ |
| Rₜ (HPLC) | 0.95 min (Z018_S04) |

### Intermediate 58

### 3-(5-((4-Methoxybenzyl)oxy)pyrazin-2-yl)-2-thioxo-2,3-dihydropyrazolo[5,1-f][1,2,4]triazin-4(1H)-one

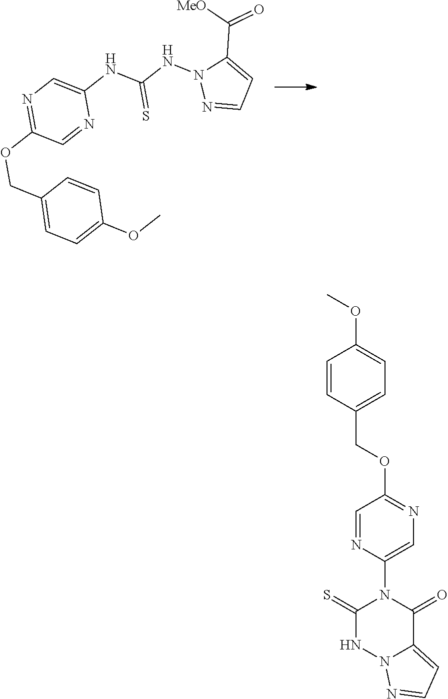 [SMILES: COC(=O)c1ccnn1NC(=S)Nc1cnc(OCc2ccc(OC)cc2)cn1 ; COc1ccc(COc2cnc(-n3c(=S)[nH]n4nccc4c3=O)cn2)cc1]

Potassium tert-butoxide (271 mg, 2.41 mmol, 1 equiv) is added to a solution of methyl 1-(3-(5-((4-methoxybenzyl)oxy)pyrazin-2-yl)thioureido)-1H-pyrazole-5-carboxylate (intermediate 57, 1.00 g, 2.41 mmol, 1 equiv) in N,N-dimethylacetamide (5 mL), and the mixture is heated at 50° C. The mixture is cooled at room temperature, and ethyl acetate and brine is added. The organic layer is separated, and the aqueous layer is extracted 3× with ethyl acetate. The combined organic layers are dried and concentrated under reduced pressure to yield the desired product.

| | |
|---|---|
| C₁₇H₁₄N₆O₃S | (MW = 382.1 g/mol) |
| ESI | 383 [M + H]⁺ |
| Rₜ (HPLC) | 0.96 min (Z018_S04) |

### Intermediate 59

### 3-(5-((4-Methoxybenzyl)oxy)pyrazin-2-yl)-2-((4-(trifluoromethyl)benzyl)thio)pyrazolo[5,1-f][1,2,4]triazin-4(3H)-one

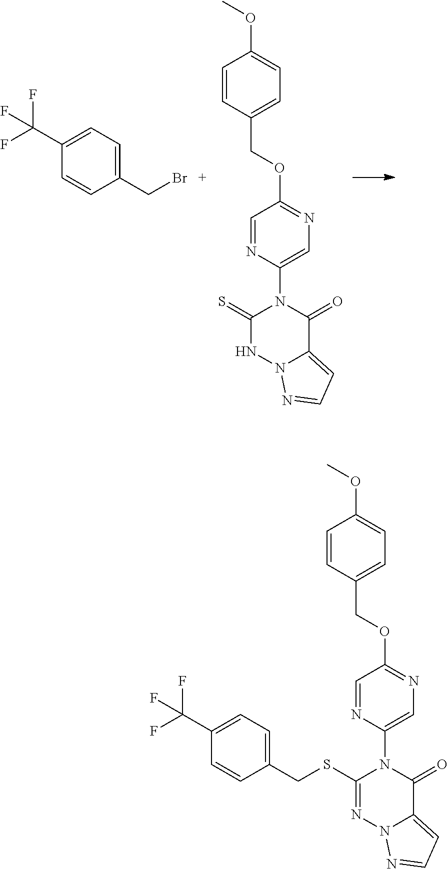

1-(Bromomethyl)-4-(trifluoromethyl)benzene (90.0 mg, 369 μmol, 0.700 equiv) is added to a solution of 3-(5-((4-methoxybenzyl)oxy)pyrazin-2-yl)-2-thioxo-2,3-dihydropyrazolo[5,1-f][1,2,4]triazin-4(1H)-one (intermediate 58, 200 mg, 523 μmol, 1 equiv) and N,N-diisopropylethylamine (181 μL, 1.05 mmol, 2.00 equiv) in N,N-dimethylacetamide (2 mL), and the mixture is stirred at room temperature for 2 h. Water and acetonitrile is added, and the mixture is lyophylized to yield the product.

| | |
|---|---|
| C₂₅H₁₉F₃N₆O₃S | (MW = 540.1 g/mol) |
| ESI | 541 [M + H]⁺ |
| Rₜ (HPLC) | 1.14 min (Z018_S04) |

### Intermediate 60

### 3-fluoro-5-isothiocyanato-2-methoxypyridine

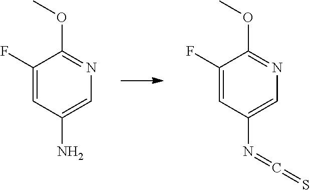

Intermediate 60 is prepared using procedures analogous to those described for intermediate 25, using appropriate starting materials.

| | |
|---|---|
| C₇H₅FN₂OS | (MW = 184.0 g/mol) |
| ESI | 185 [M + H]⁺ |
| Rₜ (HPLC) | 1.04 min (Z018_S04) |

### Intermediate 61

### Methyl 1-(3-(5-fluoro-6-methoxypyridin-3-yl)thioureido)-1H-pyrazole-5-carboxylate

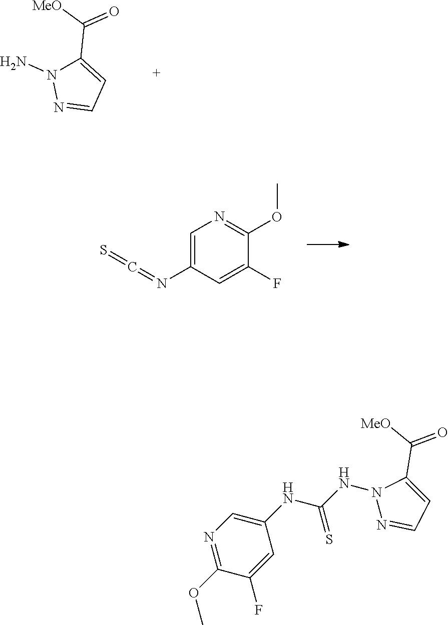 [SMILES: COC(=O)c1ccnn1N ; "+" ; COc1ncc(N=C=S)cc1F ; COC(=O)c1ccnn1NC(=S)Nc1cnc(OC)c(F)c1]

Intermediate 61 is prepared using procedures analogous to those described for intermediate 26, using appropriate starting materials.

| | |
|---|---|
| C₁₂H₁₂FN₅O₃S | (MW = 325.1 g/mol) |
| ESI | 326 [M + H]⁺ |
| Rₜ (HPLC) | 0.77 min (Z018_S04) |

### Intermediate 62

### 3-(5-Fluoro-6-methoxypyridin-3-yl)-2-((2,4,6-trifluorobenzyl)thio)pyrazolo[5,1-f][1,2,4]-triazin-4(3H)-one

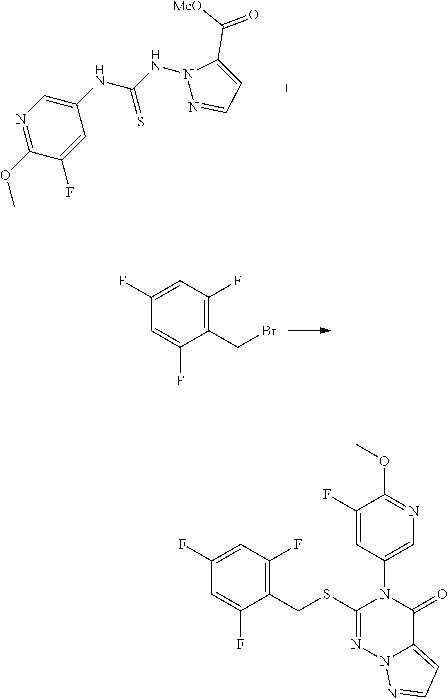 [SMILES: COC(=O)c1ccnn1NC(=S)Nc1cnc(OC)c(F)c1 ; Fc1cc(F)c(CBr)c(F)c1 ; COc1ncc(-n2c(SCc3c(F)cc(F)cc3F)nn3nccc3c2=O)cc1F]

Intermediate 62 is prepared using procedures analogous to those described for intermediate 27, using appropriate starting materials.

| | |
|---|---|
| C₁₈H₁₁F₄N₅O₂S | (MW = 437.1 g/mol) |
| ESI | 438 [M + H]⁺ |
| Rₜ (HPLC) | 1.02 min (Z018_S04) |

The following intermediates are prepared using procedures analogous to those described for intermediate 62, using appropriate starting materials. As is appreciated by those skilled in the art, the analogous examples may involve variations in general reaction conditions.

| | | | HPLC retention |
|---|---|---|---|
| | | | time [min] |
| Intermediate | Structure | ESI-MS | (method) |
| 63 | 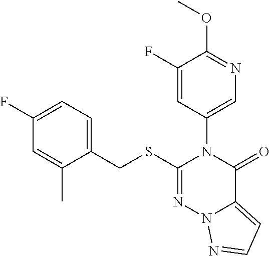 [SMILES: COc1ncc(-n2c(SCc3ccc(F)cc3C)nn3nccc3c2=O)cc1F] | 416 [M + H]⁺ | 1.07 (Z018_S04) |
| 64 | 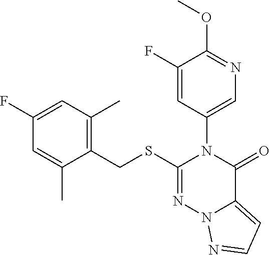 [SMILES: COc1ncc(-n2c(SCc3c(C)cc(F)cc3C)nn3nccc3c2=O)cc1F] | 430 [M + H]⁺ | 1.11 (Z018_S04) |
| 65 | 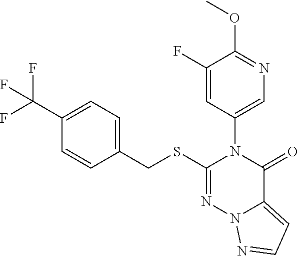 [SMILES: COc1ncc(-n2c(SCc3ccc(C(F)(F)F)cc3)nn3nccc3c2=O)cc1F] | 452 [M + H]⁺ | 1.06 (Z018_S04) |
| 66 | 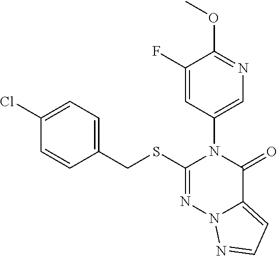 [SMILES: COc1ncc(-n2c(SCc3ccc(Cl)cc3)nn3nccc3c2=O)cc1F] | 418 [M + H]⁺ | 1.05 (Z018_S04) |
| 67 | 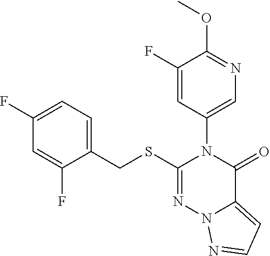 [SMILES: COc1ncc(-n2c(SCc3ccc(F)cc3F)nn3nccc3c2=O)cc1F] | 420 [M + H]⁺ | 1.02 (Z018_S04) |
| 68 | 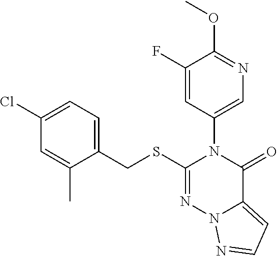 [SMILES: COc1ncc(-n2c(SCc3ccc(Cl)cc3C)nn3nccc3c2=O)cc1F] | 432 [M + H]⁺ | 1.12 (Z018_S04) |
| 69 | 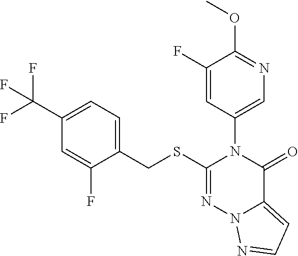 [SMILES: COc1ncc(-n2c(SCc3ccc(C(F)(F)F)cc3F)nn3nccc3c2=O)cc1F] | 470 [M + H]⁺ | 1.10 (Z018_S04) |
| 70 | 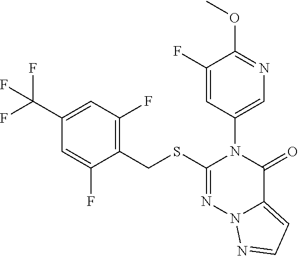 [SMILES: COc1ncc(-n2c(SCc3c(F)cc(C(F)(F)F)cc3F)nn3nccc3c2=O)cc1F] | 488 [M + H]⁺ | 1.11 (Z018_S04) |
| 71 | 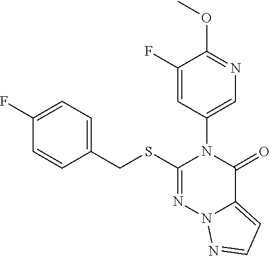 [SMILES: COc1ncc(-n2c(SCc3ccc(F)cc3)nn3nccc3c2=O)cc1F] | 402 [M + H]⁺ | 1.04 (Z018_S04) |
| 72 | 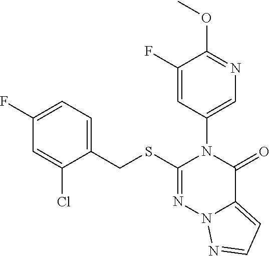 [SMILES: COc1ncc(-n2c(SCc3ccc(F)cc3Cl)nn3nccc3c2=O)cc1F] | 436 [M + H]⁺ | 1.10 (Z018_S04) |
| 73 | 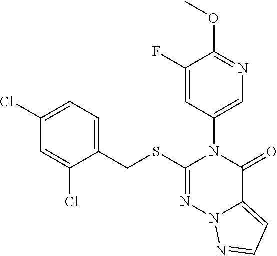 [SMILES: COc1ncc(-n2c(SCc3ccc(Cl)cc3Cl)nn3nccc3c2=O)cc1F] | 453 [M + H]⁺ | 1.14 (Z018_S04) |
| 74 | 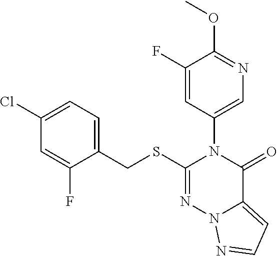 [SMILES: COc1ncc(-n2c(SCc3ccc(Cl)cc3F)nn3nccc3c2=O)cc1F] | 436 [M + H]⁺ | 1.09 (Z018_S04) |
| 75 | 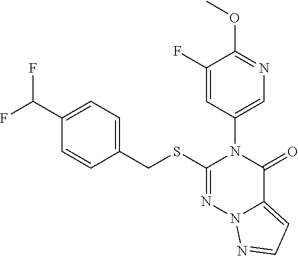 [SMILES: COc1ncc(-n2c(SCc3ccc(C(F)F)cc3)nn3nccc3c2=O)cc1F] | 434 [M + H]⁺ | 1.04 (Z018_S04) |
| 76 | 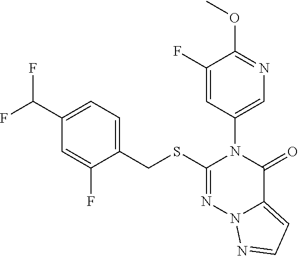 [SMILES: COc1ncc(-n2c(SCc3ccc(C(F)F)cc3F)nn3nccc3c2=O)cc1F] | 452 [M + H]⁺ | 1.05 (Z018_S04) |
| 77 | 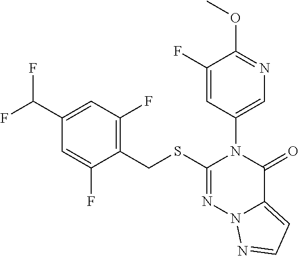 [SMILES: COc1ncc(-n2c(SCc3c(F)cc(C(F)F)cc3F)nn3nccc3c2=O)cc1F] | 470 [M + H]⁺ | 1.06 (Z018_S04) |
| 78 | 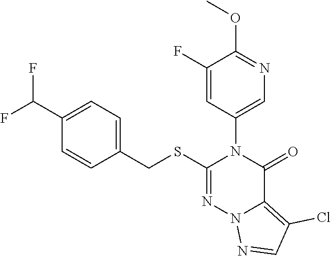 [SMILES: COc1ncc(-n2c(SCc3ccc(C(F)F)cc3)nn3ncc(Cl)c3c2=O)cc1F] | 468 [M + H]⁺ | 1.08 (Z018_S04) |
| 79 | 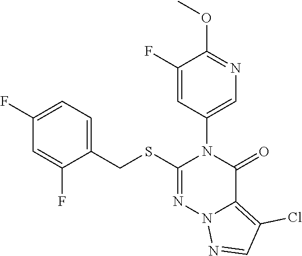 [SMILES: COc1ncc(-n2c(SCc3ccc(F)cc3F)nn3ncc(Cl)c3c2=O)cc1F] | 454 [M + H]⁺ | 1.10 (Z018_S04) |
| 80 | 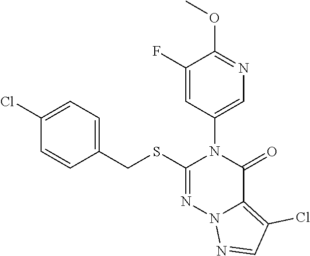 [SMILES: COc1ncc(-n2c(SCc3ccc(Cl)cc3)nn3ncc(Cl)c3c2=O)cc1F] | 452/454 [M + H]⁺ | 1.14 (Z018_S04) |
| 81 | 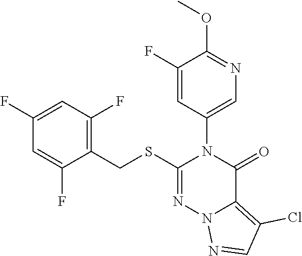 [SMILES: COc1ncc(-n2c(SCc3c(F)cc(F)cc3F)nn3ncc(Cl)c3c2=O)cc1F] | 472 [M + H]⁺ | 1.11 (Z018_S04) |
| 82 | 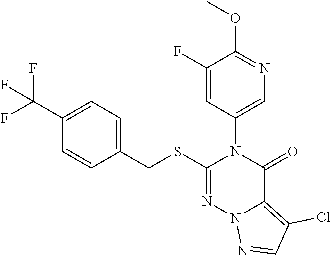 [SMILES: COc1ncc(-n2c(SCc3ccc(C(F)(F)F)cc3)nn3ncc(Cl)c3c2=O)cc1F] | 486 [M + H]⁺ | 1.14 (Z018_S04) |
| 83 | 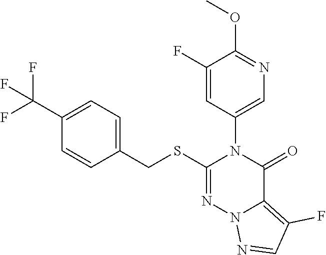 [SMILES: COc1ncc(-n2c(SCc3ccc(C(F)(F)F)cc3)nn3ncc(F)c3c2=O)cc1F] | 470 [M + H]⁺ | 1.11 (Z018_S04) |
| 84 | 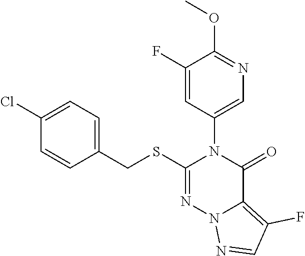 [SMILES: COc1ncc(-n2c(SCc3ccc(Cl)cc3)nn3ncc(F)c3c2=O)cc1F] | 436 [M + H]⁺ | 1.10 (Z018_S04) |
| 85 | 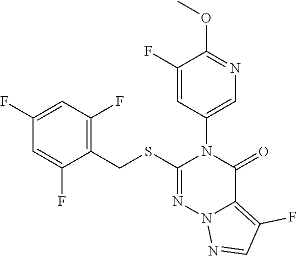 [SMILES: COc1ncc(-n2c(SCc3c(F)cc(F)cc3F)nn3ncc(F)c3c2=O)cc1F] | 456 [M + H]⁺ | 1.07 (Z018_S04) |
| 86 | 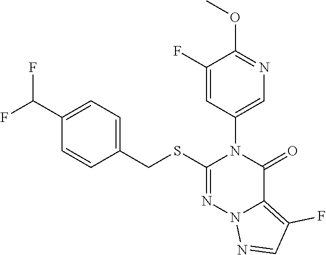 [SMILES: COc1ncc(-n2c(SCc3ccc(C(F)F)cc3)nn3ncc(F)c3c2=O)cc1F] | 452 [M + H]⁺ | 1.05 (Z018_S04) |
| 87 | 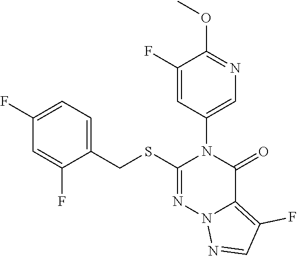 [SMILES: COc1ncc(-n2c(SCc3ccc(F)cc3F)nn3ncc(F)c3c2=O)cc1F] | 438 [M + H]⁺ | 1.07 (Z018_S04) |
| 88 | 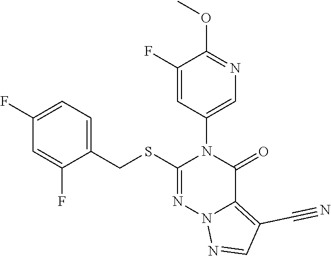 [SMILES: COc1ncc(-n2c(SCc3ccc(F)cc3F)nn3ncc(C#N)c3c2=O)cc1F] | 445 [M + H]⁺ | 0.98 (Z018_S04) |
| 89 | 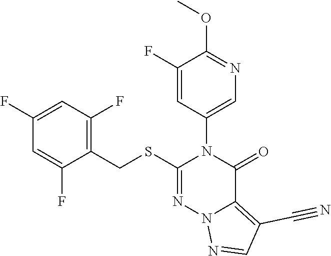 [SMILES: COc1ncc(-n2c(SCc3c(F)cc(F)cc3F)nn3ncc(C#N)c3c2=O)cc1F] | 463 [M + H]⁺ | 1.06 (Z018_S04) |
| 90 | 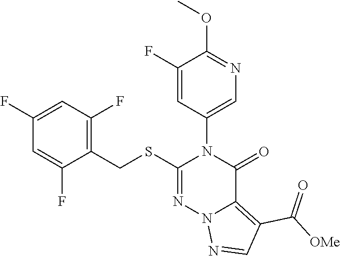 [SMILES: COC(=O)c1cnn2nc(SCc3c(F)cc(F)cc3F)n(-c3cnc(OC)c(F)c3)c(=O)c12] | 496 [M + H]⁺ | 1.06 (Z018_S04) |
| 91 | 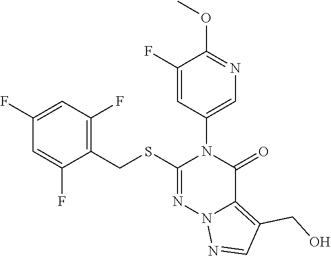 [SMILES: COc1ncc(-n2c(SCc3c(F)cc(F)cc3F)nn3ncc(CO)c3c2=O)cc1F] | 468 [M + H]⁺ | 0.99 (Z018_S04) |

### Intermediate 92

### 3-Fluoro-5-isothiocyanatopyridin-2-ol

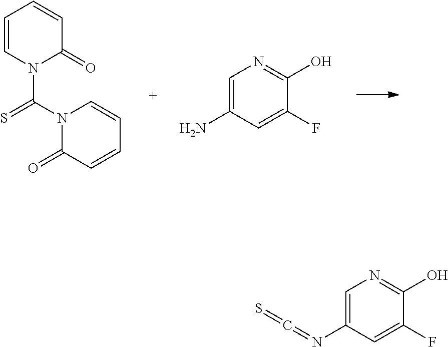 [SMILES: O=c1ccccn1C(=S)n1ccccc1=O ; Nc1cnc(O)c(F)c1 ; Oc1ncc(N=C=S)cc1F]

Intermediate 92 is prepared using procedures analogous to those described for intermediate 25, using appropriate starting materials.

| | |
|---|---|
| C₆H₃FN₂OS | (MW = 170.0 g/mol) |
| ESI | 171 [M + H]⁺ |
| Rₜ (HPLC) | 0.30 min (Z011_S03) |

### Intermediate 93

### 3-(5-Fluoro-6-hydroxypyridin-3-yl)-5-methyl-2-thioxo-2,3-dihydropyrazolo[5,1-f][1,2,4]-triazin-4(1H)-one

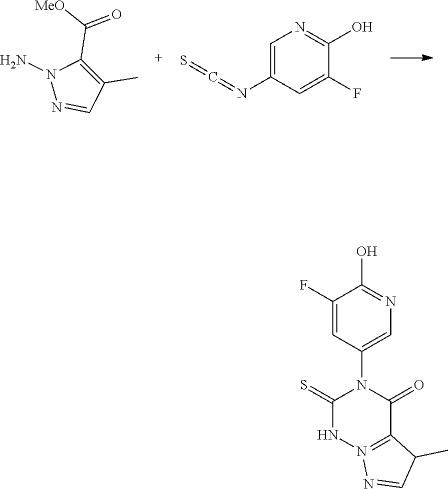

3-Fluoro-5-isothiocyanatopyridin-2-ol (intermediate 92, 173 mg, 1.02 mmol, 1 equiv) is added to a solution of methyl 1-amino-4-methyl-1H-pyrazole-5-carboxylate (intermediate 22, 157 mg, 1.01 mmol, 1 equiv) and triethylamine (282 μL, 2.02 mmol, 2.00 equiv) in acetonitrile (5 mL) and the mixture is heated at 50° C. After 16 h, the precipitate is filtered and dried to yield the desired product.

| | |
|---|---|
| C₁₁H₈FN₅O₂S | (MW = 293.0 g/mol) |
| ESI | 294 [M + H]⁺ |
| Rₜ (HPLC) | 0.24 min (X012_S01) |

### Intermediate 94

### 5-Bromo-3-(5-fluoro-6-methoxypyridin-3-yl)-2-thioxo-2,3-dihydropyrazolo[5,1-f][1,2,4]-triazin-4(1H)-one

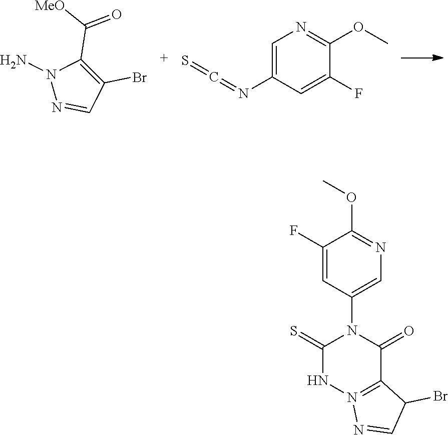

Intermediate 94 is prepared using procedures analogous to those described for intermediate 93, using appropriate starting materials.

| | |
|---|---|
| C₁₁H₇BrFN₅O₂S | (MW = 370.9 g/mol) |
| ESI | 372 [M + H]⁺ |
| Rₜ (HPLC) | 0.39 (X012_S01) |

### Intermediate 95

### 5-Bromo-3-(5-fluoro-6-methoxypyridin-3-yl)-2-((2,4,6-trifluorobenzyl)thio)pyrazolo[5,1-f][1,2,4]triazin-4(3H)-one

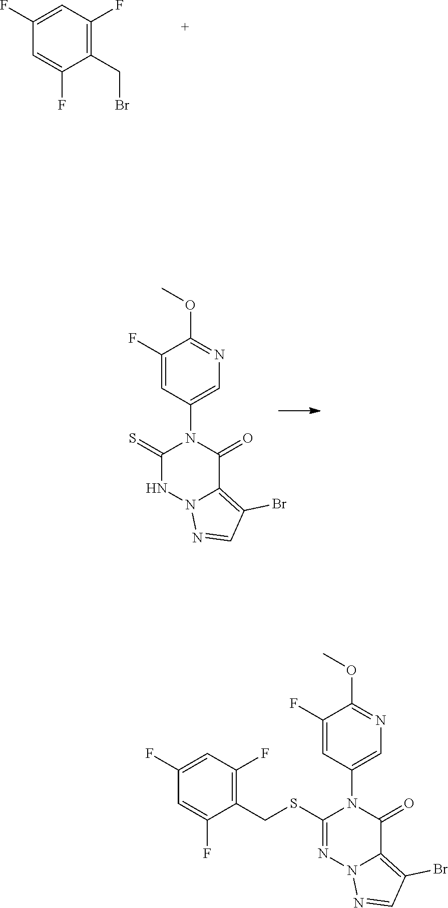 [SMILES: Fc1cc(F)c(CBr)c(F)c1 ; COc1ncc(-n2c(=S)[nH]n3ncc(Br)c3c2=O)cc1F ; COc1ncc(-n2c(SCc3c(F)cc(F)cc3F)nn3ncc(Br)c3c2=O)cc1F]

Intermediate 95 is prepared using procedures analogous to those described for intermediate 59, using appropriate starting materials.

| | |
|---|---|
| C₁₈H₁₀BrF₄N₅O₂S | (MW = 515.0 g/mol) |
| ESI | 516 [M + H]⁺ |
| Rₜ (HPLC) | 0.73 (X012_S01) |

### Intermediate 96

### 5-Cyclopropyl-3-(5-fluoro-6-methoxypyridin-3-yl)-2-((2,4,6-trifluorobenzyl)thio)pyrazolo[5,1-f][1,2,4]triazin-4(3H)-one

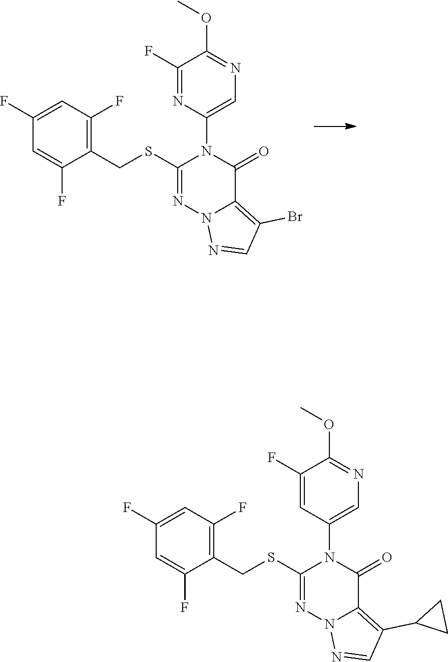 [SMILES: COc1ncc(-n2c(SCc3c(F)cc(F)cc3F)nn3ncc(Br)c3c2=O)nc1F ; COc1ncc(-n2c(SCc3c(F)cc(F)cc3F)nn3ncc(C4CC4)c3c2=O)cc1F]

A mixture of 5-bromo-3-(5-fluoro-6-methoxypyridin-3-yl)-2-((2,4,6-trifluorobenzyl)thio)pyrazolo[5,1-f][1,2,4]triazin-4(3H)-one (intermediate 95, 39.0 mg, 76.0 μmol, 1 equiv) cyclopropylboronic acid (20.0 mg, 226 μmol, 3.00 equiv) XPhosPdG4 (8.00 mg, 9.00 μmol, 0.100 equiv), cesium carbonate (99.0 mg, 304 μmol, 4.00 equiv) in 1,4-dioxane (0.6 mL) is heated at 100° C. After 1 h water is added, and the mixture is extracted with dichloromethane. The combined organic layers are concentrated under reduced pressure, and the residue is purified by reversed phase HPLC (XBridge™-C18, gradient of acetonitrile in water, 0.1% TFA) to yield the desired product.

| | |
|---|---|
| C₂₁H₁₅F₄N₅O₂S | (MW = 477.1 g/mol) |
| ESI | 478 [M + H]⁺ |
| Rₜ (HPLC) | 0.77 (X012_S01) |

### Preparation of Final Compounds

### Example 1

### 3-(5-Hydroxypyrazin-2-yl)-5-nitro-2-((2,4,6-trifluorobenzyl)thio)pyrazolo[5,1-f][1,2,4]triazin-4(3H)-one

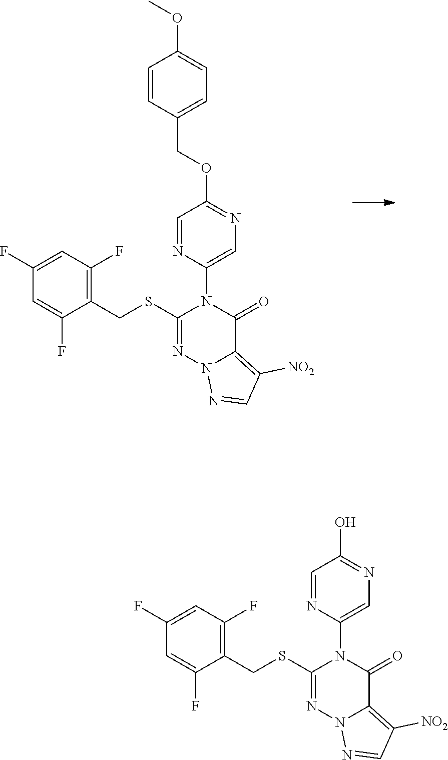 [SMILES: COc1ccc(COc2cnc(-n3c(SCc4c(F)cc(F)cc4F)nn4ncc([N+](=O)[O-])c4c3=O)cn2)cc1 ; O=c1c2c([N+](=O)[O-])cnn2nc(SCc2c(F)cc(F)cc2F)n1-c1cnc(O)cn1]

A mixture of 3-(5-((4-methoxybenzyl)oxy)pyrazin-2-yl)-5-nitro-2-((2,4,6-trifluorobenzyl)thio)pyrazolo[5,1-f][1,2,4]triazin-4(3H)-one (intermediate 27, 100 mg, 175 μmol, 1 equiv) and Amberlyst® 15(H) (68.5 mg, 315 μmol, 1.80 equiv) in methanol (2 mL) is heated at 60° C. After 1 h, NN-dimethylacetamide is added, the mixture is filtered, and the filtrate is purified by reversed phase HPLC (Agilent Sunfire™-C18, gradient of acetonitrile in water, 0.1% TFA) to yield the desired product.

| | |
|---|---|
| C₁₆H₈F₃N₇O₄S | (MW = 451.0 g/mol) |
| ESI | 452 [M + H]⁺ |
| Rₜ (HPLC) | 0.91 min (Z018_S04) |

### Example 2

### 5-Amino-3-(5-hydroxypyrazin-2-yl)-2-((2,4,6-trifluorobenzyl)thio)pyrazolo[5,1-f][1,2,4]-triazin-4(3H)-one

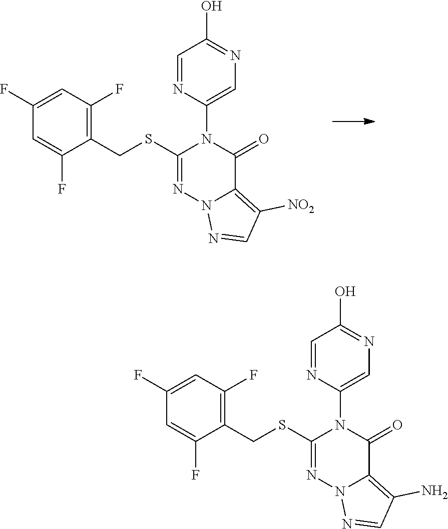 [SMILES: O=c1c2c([N+](=O)[O-])cnn2nc(SCc2c(F)cc(F)cc2F)n1-c1cnc(O)cn1 ; Nc1cnn2nc(SCc3c(F)cc(F)cc3F)n(-c3cnc(O)cn3)c(=O)c12]

A mixture of 3-(5-hydroxypyrazin-2-yl)-5-nitro-2-((2,4,6-trifluorobenzyl)thio)pyrazolo[5,1-f][1,2,4]triazin-4(3H)-one (example 1, 25.0 mg, 53.0 μmol, 1 equiv) and Raney-Nickel (5 mg) in tetrahydrofuran (2 mL) is stirred under hydrogen atmosphere (3 bar). After 4 h, the mixture is filtered, and the filtrate is concentrated to yield the product.

| | |
|---|---|
| C₁₆H₁₀F₃N₇O₂S | (MW = 421.1 g/mol) |
| ESI | 422 [M + H]⁺ |
| Rₜ (HPLC) | 0.79 min (Z018_S04) |

### Example 3 and 4

### 5-(Fluoromethyl)-3-(5-hydroxypyrazin-2-yl)-2-((4-(trifluoromethyl)benzyl)thio)pyrazolo[5,1-f][1,2,4]triazin-4(3H)-one and 3-(5-hydroxypyrazin-2-yl)-5-(methoxymethyl)-2-((4-(trifluoromethyl)benzyl)thio)pyrazolo[5,1-f][1,2,4]triazin-4(3H)-one

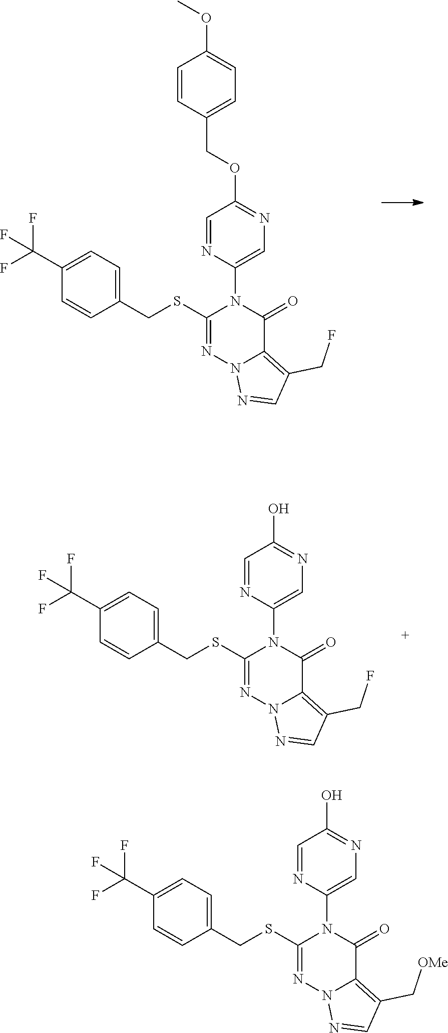 [SMILES: COc1ccc(COc2cnc(-n3c(SCc4ccc(C(F)(F)F)cc4)nn4ncc(CF)c4c3=O)cn2)cc1 ; O=c1c2c(CF)cnn2nc(SCc2ccc(C(F)(F)F)cc2)n1-c1cnc(O)cn1 ; COCc1cnn2nc(SCc3ccc(C(F)(F)F)cc3)n(-c3cnc(O)cn3)c(=O)c12]

A mixture of 5-(fluoromethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 38, 10.0 mg, 17.0 μmol, 1 equiv) and Amberlyst® 15(H) (19 mg, 87.0 μmol, 5.00 equiv) in methanol (0.5 mL) is heated at 70° C. After 2 h, the reaction mixture is filtered and the filtrate is purified by reversed phase HPLC (XBridge™-C18, gradient of acetonitrile in water, 0.1% TFA) to yield the desired products.

### Example 3

| | |
|---|---|
| C₁₈H₁₂F₄N₆O₂S | (MW = 452.1 g/mol) |
| ESI | 453 [M + H]⁺ |
| Rₜ (HPLC) | 0.58 min (X012_S01) |

### Example 4

| | |
|---|---|
| C₁₉H₁₅F₃N₆O₃S | (MW = 464.1 g/mol) |
| ESI | 463 [M − H]⁻ |
| Rₜ (HPLC) | 0.57 min (X012_S01) |

The following examples are prepared using procedures analogous to those described for examples 3 and 4, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 5 | 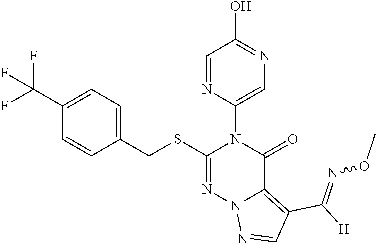 [SMILES: CON=Cc1cnn2nc(SCc3ccc(C(F)(F)F)cc3)n(-c3cnc(O)cn3)c(=O)c12] | 478 [M + H]⁺ | 0.62 (X012_S01) |
| 6 | 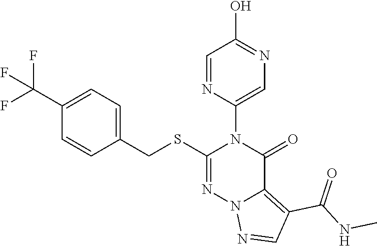 [SMILES: CNC(=O)c1cnn2nc(SCc3ccc(C(F)(F)F)cc3)n(-c3cnc(O)cn3)c(=O)c12] | 478 [M + H]⁺ | 0.75 (X018_S03) |
| 7 | 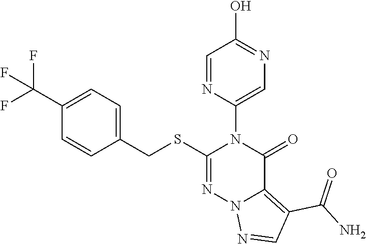 [SMILES: NC(=O)c1cnn2nc(SCc3ccc(C(F)(F)F)cc3)n(-c3cnc(O)cn3)c(=O)c12] | 464 [M + H]⁺ | 0.71 (X018_S03) |
| 8 | 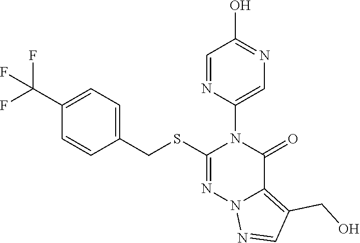 [SMILES: O=c1c2c(CO)cnn2nc(SCc2ccc(C(F)(F)F)cc2)n1-c1cnc(O)cn1] | 451 [M + H]⁺ | 0.49 (X012_S01) |
| 9 | 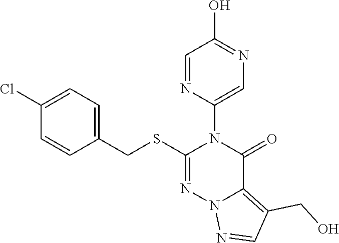 [SMILES: O=c1c2c(CO)cnn2nc(SCc2ccc(Cl)cc2)n1-c1cnc(O)cn1] | 415 [M − H]⁻ | 0.46 (X012_S01) |
| 10 | 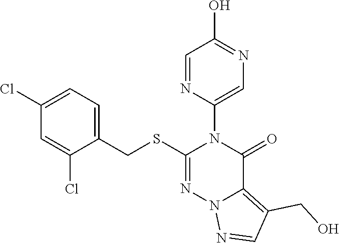 [SMILES: O=c1c2c(CO)cnn2nc(SCc2ccc(Cl)cc2Cl)n1-c1cnc(O)cn1] | 451 [M + H]⁺ | 0.51 (X012_S01) |
| 11 | 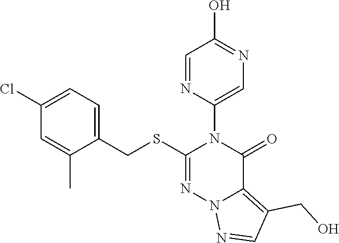 [SMILES: Cc1cc(Cl)ccc1CSc1nn2ncc(CO)c2c(=O)n1-c1cnc(O)cn1] | 432 [M + H]⁺ | 0.42 (X012_S01) |
| 12 | 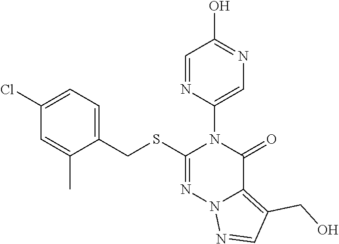 [SMILES: Cc1cc(Cl)ccc1CSc1nn2ncc(CO)c2c(=O)n1-c1cnc(O)cn1] | 433 [M − H]⁻ | 0.49 (X012_S01) |

### Example 13 and 14

### 3-(5-Hydroxypyrazin-2-yl)-4-oxo-2-((4-(trifluoromethyl)benzyl)thio)-3,4-dihydropyrazolo[5,1-f][1,2,4]triazine-5-carbaldehyde Oxime

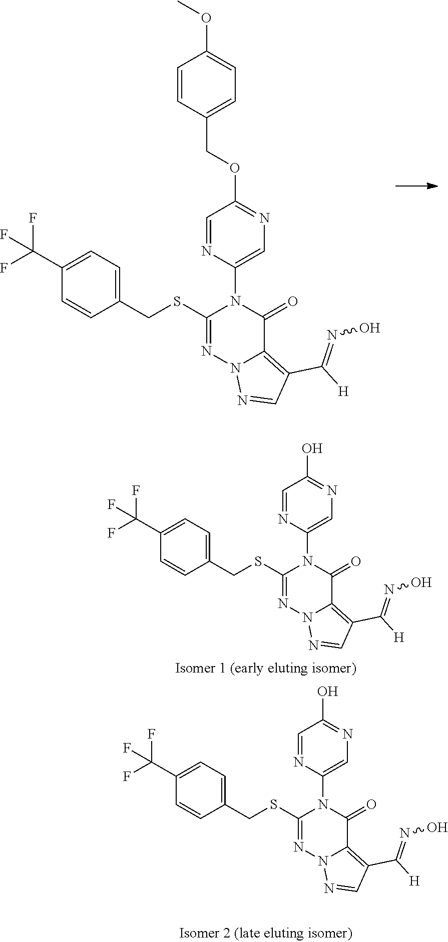 [SMILES: COc1ccc(COc2cnc(-n3c(SCc4ccc(C(F)(F)F)cc4)nn4ncc(C=NO)c4c3=O)cn2)cc1 ; O=c1c2c(C=NO)cnn2nc(SCc2ccc(C(F)(F)F)cc2)n1-c1cnc(O)cn1 ; O=c1c2c(C=NO)cnn2nc(SCc2ccc(C(F)(F)F)cc2)n1-c1cnc(O)cn1]

A mixture of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde oxime (intermediate 45, 96.0 mg, 123 μmol, 1.0 eq) and Amberlyst® 15(H) (134 mg, 616 μmol, 5.0 equiv) in methanol (1.0 mL) is heated at 50° C. for 4 h. The mixture is filtered, and the filtrate is purified by reversed phase HPLC (XBridge™-C18, gradient of acetonitrile in water, 0.1% TFA) to yield the desired products.

### Example 13 (Isomer 1, Early Eluting Isomer)

| | |
|---|---|
| C₁₈H₁₂F₃N₇O₃S | (MW = 463.4 g/mol) |
| ESI | 464 [M + H]⁺ |
| Rₜ (HPLC) | 0.52 min (X012_S01) |

### Example 14 (Isomer 1, Late Eluting Isomer)

| | |
|---|---|
| C₁₈H₁₂F₃N₇O₃S | (MW = 463.4 g/mol) |
| ESI | 464 [M + H]⁺ |
| Rₜ (HPLC) | 0.53 min (X012_S01) |

### Example 15

### 3-(5-Hydroxypyrazin-2-yl)-4-oxo-2-((4-(trifluoromethyl)benzyl)thio)-3,4-dihydropyrazolo[5,1-f][1,2,4]triazine-5-carbaldehyde

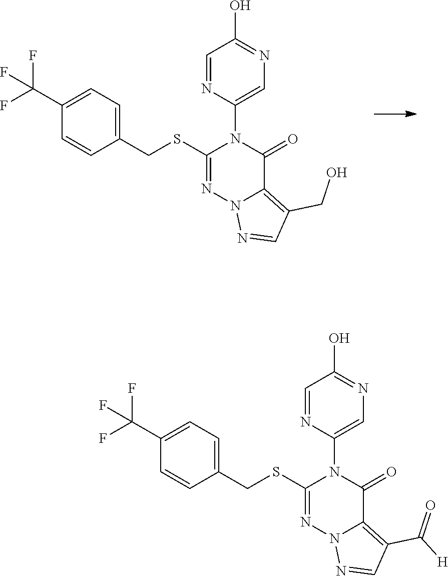 [SMILES: O=c1c2c(CO)cnn2nc(SCc2ccc(C(F)(F)F)cc2)n1-c1cnc(O)cn1 ; O=Cc1cnn2nc(SCc3ccc(C(F)(F)F)cc3)n(-c3cnc(O)cn3)c(=O)c12]

Dess-Martin Periodinane (35.0 mg, 78.0 μmol, 1.10 equiv) is added to a solution of 5-(hydroxymethyl)-3-(5-hydroxypyrazin-2-yl)-2-((4-(trifluoromethyl)benzyl)thio)pyrazolo[5,1-f][1,2,4]triazin-4(3H)-one (example 8, 32.0 mg, 71.0 μmol, 1 equiv) in dichloromethane (1 mL) at 0° C. and the reaction mixture is allowed to warm at room temperature. After 18 h, the reaction mixture is cooled to 0° C., and additional DMP (10.0 mg, 22.0 μmol, 0.300 equiv) is added. The reaction mixture is allowed to warm at room temperature and after 1 h, water is added, and the mixture is extracted with dichloromethane. The combined organic layers are filtered through a phase transfer filter, and the filtrate is concentrated under reduced pressure. The residue is purified by reversed phase HPLC (X-Bridge-C18, gradient of acetonitrile in water, 0.1% TFA) to yield the desired product.

| | |
|---|---|
| C₁₈H₁₁F₃N₆O₃S | (MW = 448.1 g/mol) |
| ESI | 449 [M + H]⁺ |
| Rₜ (HPLC) | 0.55 min (X012_S01) |

### Example 16

### 5-Ethynyl-3-(5-hydroxypyrazin-2-yl)-2-((4-(trifluoromethyl)benzyl)thio)pyrazolo[5,1-f][1,2,4]triazin-4(3H)-one

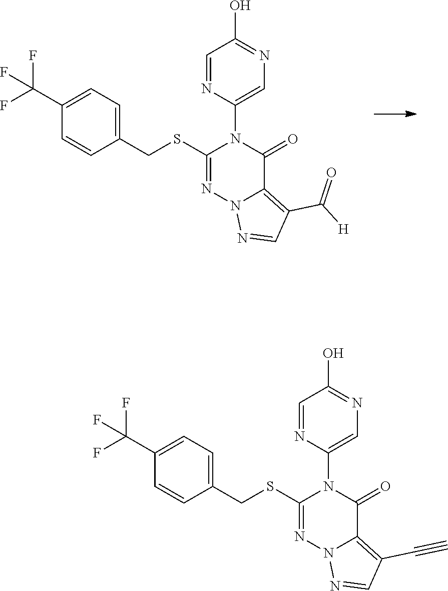 [SMILES: O=Cc1cnn2nc(SCc3ccc(C(F)(F)F)cc3)n(-c3cnc(O)cn3)c(=O)c12 ; C#Cc1cnn2nc(SCc3ccc(C(F)(F)F)cc3)n(-c3cnc(O)cn3)c(=O)c12]

Potassium carbonate (27.0 mg, 195 μmol, 1.10 equiv) is added to a mixture of 3-(5-hydroxypyrazin-2-yl)-4-oxo-2-((4-(trifluoromethyl)benzyl)thio)-3,4-dihydropyrazolo[5,1-f][1,2,4]triazine-5-carbaldehyde (example 15, 77.0 mg, 172 μmol, 1 equiv) and dimethyl (1-diazo-2-oxopropyl)-phosphonate (10% in acetonitrile, 454 μL, 189 μmol, 1.10 equiv) in a mixture of methanol (0.7 mL) and tetrahydrofuran (1.7 mL). After 30 min, additional potassium carbonate (27.0 mg, 195 μmol, 1.10 equiv) is added. After 16 h, water is added, and the reaction mixture is neutralized with aqueous hydrochloric acid (1 M). The aqueous layer is extracted with ethyl acetate. The combined organic layers are dried over magnesium sulphate, the dried solution is filtered, and the filtrate is concentrated under reduced pressure. The residue is recrystallized from acetone/water (80:20) to provide the product.

| | |
|---|---|
| C₁₉H₁₁F₃N₆O₂S | (MW = 444.1 g/mol) |
| ESI | 445 [M + H]⁺ |
| Rₜ (HPLC) | 0.59 min (X012_S01) |

### Example 17

### 5-(1-Hydroxyethyl)-3-(5-hydroxypyrazin-2-yl)-2-((4-(trifluoromethyl)benzyl)thio)pyrazolo[5,1-f][1,2,4]triazin-4(3H)-one

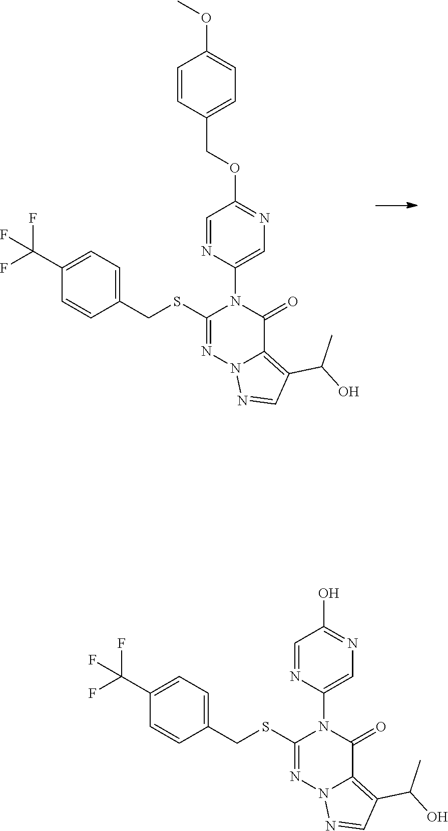 [SMILES: COc1ccc(COc2cnc(-n3c(SCc4ccc(C(F)(F)F)cc4)nn4ncc(C(C)O)c4c3=O)cn2)cc1 ; CC(O)c1cnn2nc(SCc3ccc(C(F)(F)F)cc3)n(-c3cnc(O)cn3)c(=O)c12]

Example 17 is prepared using procedures analogous to those described for example 13, using appropriate starting materials

| | |
|---|---|
| C₁₉H₁₅F₃N₆O₃S | (MW = 464.1 g/mol) |
| ESI | 465 [M + H]⁺ |
| Rₜ (HPLC) | 0.52 min (X012_S01) |

### Example 18

### 5-Acetyl-3-(5-hydroxypyrazin-2-yl)-2-((4-(trifluoromethyl)benzyl)thio)pyrazolo[5,1-f][1,2,4]triazin-4(3H)-one

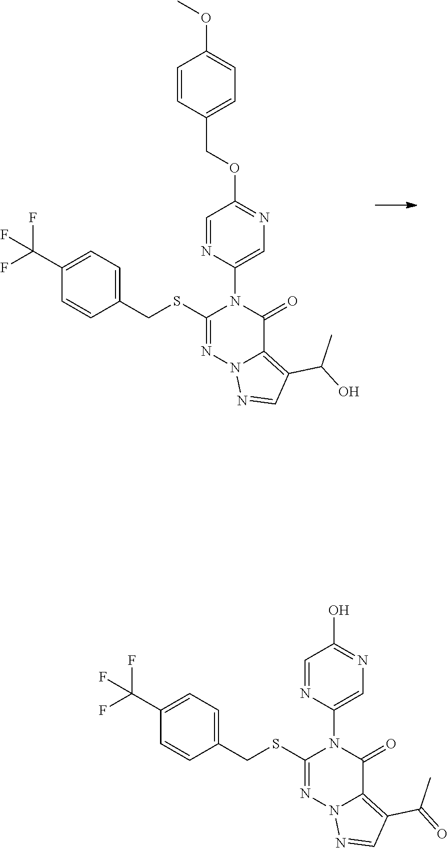 [SMILES: COc1ccc(COc2cnc(-n3c(SCc4ccc(C(F)(F)F)cc4)nn4ncc(C(C)O)c4c3=O)cn2)cc1 ; CC(=O)c1cnn2nc(SCc3ccc(C(F)(F)F)cc3)n(-c3cnc(O)cn3)c(=O)c12]

Dess-Martin Periodinane (14.0 mg, 33.0 μmol, 1 equiv) is added to a solution of 5-(1-hydroxyethyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 41, 19.0 mg, 33.0 μmol, 1 equiv) in dichloromethane (0.5 mL) at 0° C. and the reaction mixture is allowed to warm at room temperature. After 45 min, water is added, and the mixture is extracted with dichloromethane. The combined organic layers are filtered through a phase transfer filter, and the filtrate is concentrated under reduced pressure. The residue is dissolved in a mixture of N,N-dimethylformamide (0.1 mL) and methanol (0.7 mL), followed by the addition of Amberlyst® 15(H) (36.0 mg, 166 μmol, 5.10 equiv) and the mixture is heated at 50° C. After 2 h the reaction mixture is filtered and the filtrate is purified by reversed phase HPLC (X-Bridge-C18, gradient of acetonitrile in water, 0.1% TFA) to yield the desired product.

| | |
|---|---|
| C₁₉H₁₃F₃N₆O₃S | (MW = 462.1 g/mol)F, CN, Me |
| ESI | 463 [M + H]⁺ |
| Rₜ (HPLC) | 0.55 min (X012_S01) |

### Example 19

### 3-(5-Hydroxypyrazin-2-yl)-2-((4-(trifluoromethyl)benzyl)thio)-5-vinylpyrazolo[5,1-f][1,2,4]triazin-4(3H)-one

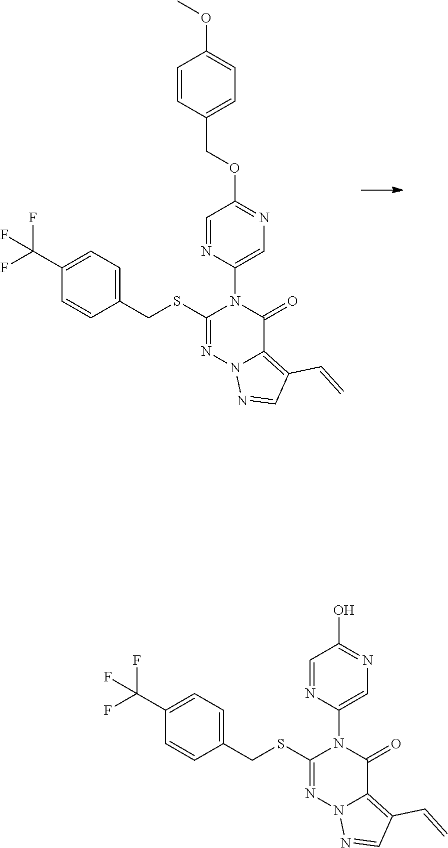 [SMILES: C=Cc1cnn2nc(SCc3ccc(C(F)(F)F)cc3)n(-c3cnc(OCc4ccc(OC)cc4)cn3)c(=O)c12 ; C=Cc1cnn2nc(SCc3ccc(C(F)(F)F)cc3)n(-c3cnc(O)cn3)c(=O)c12]

A mixture of 3-(5-((4-methoxybenzyl)oxy)pyrazin-2-yl)-4-oxo-2-((4-(trifluoromethyl)benzyl)thio)-3,4-dihydropyrazolo[5,1-f][1,2,4]triazine-5-carbaldehyde (intermediate 46, 120.0 mg, 212 μmol, 1.0 equiv) and Amberlyst® 15(H) (240 mg, 1.10 mmol, 5.2 equiv) in methanol (6.0 mL) is stirred at 50° C. for 5. The mixture is cooled at room temperature and stirred over night. Another portion of Amberlyst® 15(H) (240 mg, 1.10 mmol, 5.2 equiv) is added and the mixture is stirred at 50° C. for 4 h. The mixture is cooled at room temperature and stirred over night. The mixture is purified by reversed phase HPLC (XBridge™-C18, gradient of acetonitrile in water, 0.1% TFA) to yield the desired product.

| | |
|---|---|
| C₁₉H₁₃F₃N₆O₂S | (MW = 446.4 g/mol) |
| ESI | 447 [M + H]⁺ |
| Rₜ (HPLC) | 0.61 min (X012_S01) |

### Examples 20 and 21

### (E)-5-(2-Fluorovinyl)-3-(5-hydroxypyrazin-2-yl)-2-((4-(trifluoromethyl)benzyl)thio)pyrazolo[5,1-f][1,2,4]triazin-4(3H)-one and (Z)-5-(2-fluorovinyl)-3-(5-hydroxypyrazin-2-yl)-2-((4-(trifluoromethyl)benzyl)thio)pyrazolo[5,1-f][1,2,4]triazin-4(3H)-one

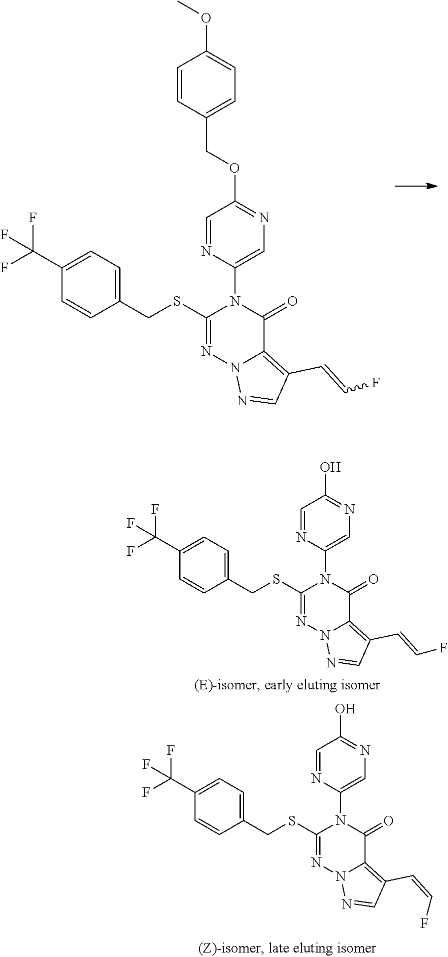 [SMILES: COc1ccc(COc2cnc(-n3c(SCc4ccc(C(F)(F)F)cc4)nn4ncc(C=CF)c4c3=O)cn2)cc1 ; O=c1c2c(/C=C/F)cnn2nc(SCc2ccc(C(F)(F)F)cc2)n1-c1cnc(O)cn1 ; O=c1c2c(/C=C\F)cnn2nc(SCc2ccc(C(F)(F)F)cc2)n1-c1cnc(O)cn1]

A mixture of 5-(2-fluorovinyl)-3-(5-hydroxypyrazin-2-yl)-2-((4-(trifluoromethyl)benzyl)thio)pyrazolo[5,1-f][1,2,4]triazin-4(3H)-one (intermediate 47, 8:2 mixture of (E)- and (Z)isomers, 40.0 mg, 68 μmol, 1.0 equiv) and Amberlyst® 15(H) (75 mg, 345 μmol, 5.0 equiv) in methanol (1.0 mL) is stirred at 50° C. for 5 h. The mixture is cooled at room temperature and stirred over night. Another portion of Amberlyst® 15(H) (75 mg, 345 μmol, 5.0 equiv) is added, and the mixture is stirred at 50° C. for 2 h. The mixture is diluted with acetonitrile and purified by reversed phase HPLC (XBridge™-C18, gradient of acetonitrile in water, 0.1% TFA) to yield the desired products.

### Example 20 ((E)-Isomer, Early Eluting Isomer)

| | |
|---|---|
| C₁₉H₁₂F₄N₆O₂S | (MW = 464.1 g/mol) |
| ESI | 465 [M + H]⁺ |
| Rₜ (HPLC) | 0.61 min (X012_S01) |

### Example 21 ((Z)-Isomer, Late Eluting Isomer)

| | |
|---|---|
| C₁₉H₁₂F₄N₆O₂S | (MW = 464.1 g/mol) |
| ESI | 465 [M + H]⁺ |
| Rₜ (HPLC) | 0.62 min (X012_S01) |

### Example 22

### 5-(2,2-Difluorovinyl)-3-(5-hydroxypyrazin-2-yl)-2-((4-(trifluoromethyl)benzyl)thio)pyrazolo[5,1-f][1,2,4]triazin-4(3H)-one

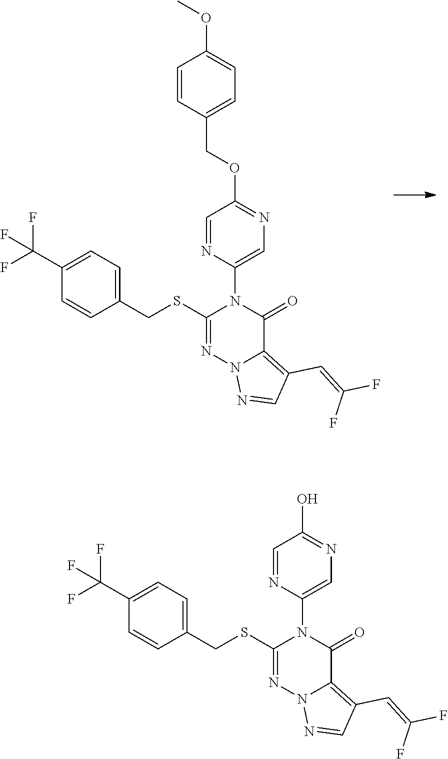 [SMILES: COc1ccc(COc2cnc(-n3c(SCc4ccc(C(F)(F)F)cc4)nn4ncc(C=C(F)F)c4c3=O)cn2)cc1 ; O=c1c2c(C=C(F)F)cnn2nc(SCc2ccc(C(F)(F)F)cc2)n1-c1cnc(O)cn1]

A mixture of 5-(2,2-difluorovinyl)-3-(5-((4-methoxybenzyl)oxy)pyrazin-2-yl)-2-((4-(trifluoromethyl)benzyl)thio)pyrazolo[5,1-f][1,2,4]triazin-4(3H)-one (intermediate 48, 102 mg, 169 μmol, 1.0 equiv) and Amberlyst® 15(H) (25 mg, 115 μmol, 0.7 equiv) in methanol (1.0 mL) is stirred at reflux for 1 h. The mixture is cooled at room temperature and filtered. The filtrate is purified by reversed phase HPLC (XBridge™-C18, gradient of acetonitrile in water, 0.1% TFA) to yield the desired product.

| | |
|---|---|
| C₁₉H₁₁F₅N₆O₂S | (MW = 482.1 g/mol) |
| ESI | 483 [M + H]⁺ |
| Rₜ (HPLC) | 0.65 min (X012_S01) |

### Example 23

### 2-((4-(2,2-Difluorovinyl)benzyl)thio)-5-fluoro-3-(5-hydroxypyrazin-2-yl)pyrazolo[5,1-f][1,2,4]triazin-4(3H)-one

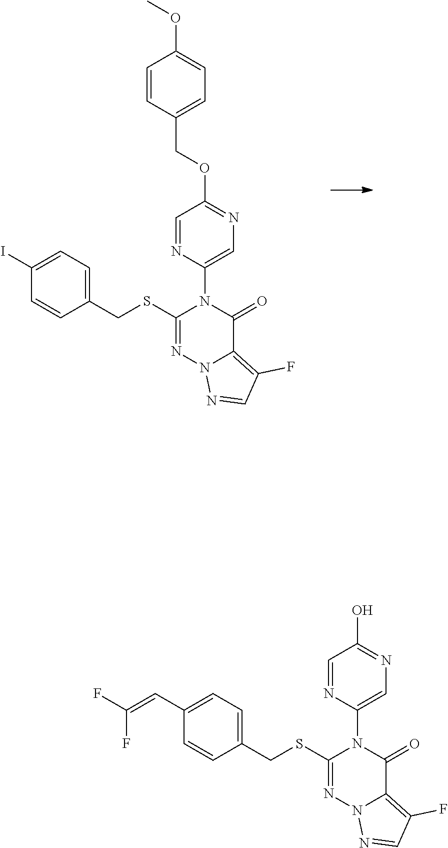 [SMILES: COc1ccc(COc2cnc(-n3c(SCc4ccc(I)cc4)nn4ncc(F)c4c3=O)cn2)cc1 ; O=c1c2c(F)cnn2nc(SCc2ccc(C=C(F)F)cc2)n1-c1cnc(O)cn1]

Aqueous potassium carbonate solution (2 M, 122 μL, 244 μmol, 3.0 equiv) and [1,1′-bis(diphenylphosphino)ferrocene]dichloropalladium(II) (7 mg, 9 μmol, 0.1 equiv) is added to a mixture of 5-fluoro-2-((4-iodobenzyl)thio)-3-(5-((4-methoxybenzyl)oxy)pyrazin-2-yl)pyrazolo[5,1-f][1,2,4]triazin-4(3H)-one (intermediate 49, 50.0 mg, 81 μmol, 1.0 equiv) and 2-(2,2-difluorovinyl)-4,4,5,5-tetramethyl-1,3,2-dioxaborolane (46 mg, 243 μmol, 3.0 equiv) in 1,4-dioxane (2.0 mL), and the mixture is stirred at 50° C. for 48 h. Water and ethyl acetate is added, and the phases are separated. The organic layer is washed with water and concentrated under reduced pressure. The residue is treated with MeOH (5 mL) and Amberlyst® 15(H) (50 mg, 230 μmol, 2.8 equiv), and the mixture is stirred at reflux for 3 h. The mixture is filtered, and the filtrate is concentrated under reduced pressure. The residue is purified by SFC (Waters Viridis® BEH OBD, gradient of supercritical CO₂ in MeOH) to yield the desired product.

| | |
|---|---|
| C₁₈H₁₁F₃N₆O₂S | (MW = 432.1 g/mol) |
| ESI | 433 [M + H]⁺ |
| Rₜ (HPLC) | 0.94 min (Z018_S04) |

### Example 24

### 5-Fluoro-3-(5-hydroxypyrazin-2-yl)-2-((4-(prop-1-yn-1-yl)benzyl)thio)pyrazolo[5,1-f][1,2,4]triazin-4(3H)-one

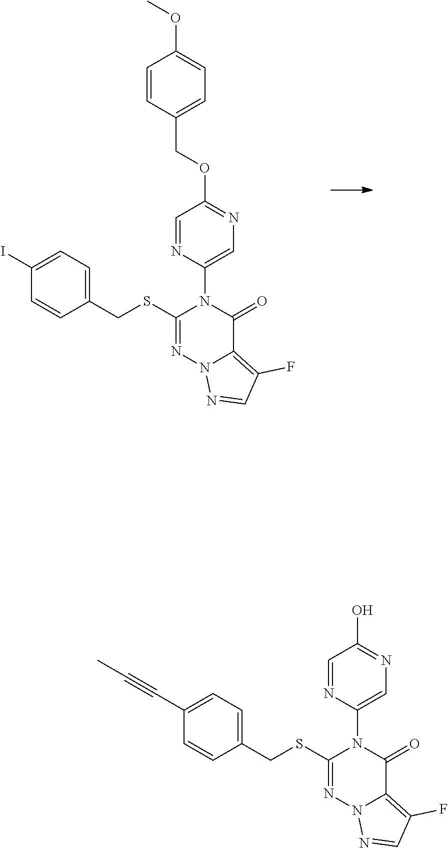 [SMILES: COc1ccc(COc2cnc(-n3c(SCc4ccc(I)cc4)nn4ncc(F)c4c3=O)cn2)cc1 ; CC#Cc1ccc(CSc2nn3ncc(F)c3c(=O)n2-c2cnc(O)cn2)cc1]

XPhosPdG4 (14.0 mg, 15 μmol, 0.1 equiv) and CuI (5.9 mg, 31 μmol, 0.2 equiv) is added to a mixture of 5-fluoro-2-((4-iodobenzyl)thio)-3-(5-((4-methoxybenzyl)oxy)pyrazin-2-yl)pyrazolo[5,1-f][1,2,4]triazin-4(3H)-one (intermediate 49, 100.0 mg, 154 μmol, 1.0 equiv), propyne (1 M in THF, 490 μL, 490 μmol, 3.2 equiv) and cesium carbonate (55 mg, 170 μmol, 1.1 equiv). The mixture is heated at reflux over night and then cooled at room temperature. Water and ethyl acetate is added, and the phases are separated. The phases are separated, and the organic layer is washed with water and concentrated under reduced pressure. The residue is treated with MeOH (2 mL) and Amberlyst® 15(H) (100 mg, 460 μmol, 3.0 equiv) and heated at reflux for 2 h. The mixture is cooled at room temperature and filtered. The filtrate is concentrated under reduced pressure, and the residue is purified by reversed phase HPLC (Agilent Sunfire™-C18, gradient of acetonitrile in water, 0.1% TFA) to yield the desired product.

| | |
|---|---|
| C₁₉H₁₃FN₆O₂S | (MW = 408.1 g/mol) |
| ESI | 409 [M + H]⁺ |
| Rₜ (HPLC) | 0.92 min (Z018_S04) |

### Example 25

### 5-Fluoro-3-(6-fluoro-5-hydroxypyrazin-2-yl)-2-((4-(trifluoromethyl)benzyl)thio)pyrazolo[5,1-f][1,2,4]triazin-4(3H)-one

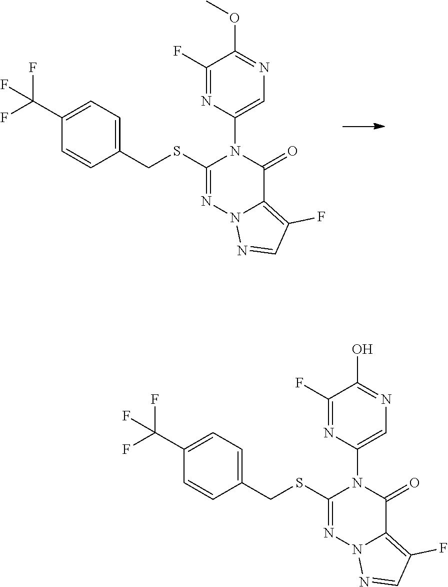 [SMILES: COc1ncc(-n2c(SCc3ccc(C(F)(F)F)cc3)nn3ncc(F)c3c2=O)nc1F ; O=c1c2c(F)cnn2nc(SCc2ccc(C(F)(F)F)cc2)n1-c1cnc(O)c(F)n1]

Example 25 is prepared using procedures analogous to those described for example 127, using appropriate starting materials.

| | |
|---|---|
| C₁₇H₉F₅N₆O₂S | (MW = 456.0 g/mol) |
| ESI | 457 [M + H]⁺ |
| Rₜ (HPLC) | 0.98 min (Z018_S04) |

### Example 26

### 5-Fluoro-3-(3-fluoro-5-hydroxypyrazin-2-yl)-2-((4-(trifluoromethyl)benzyl)thio)pyrazolo[5,1-f][1,2,4]triazin-4(3H)-one

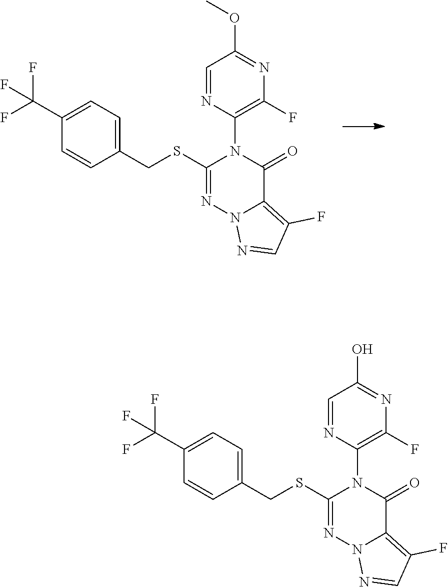 [SMILES: COc1cnc(-n2c(SCc3ccc(C(F)(F)F)cc3)nn3ncc(F)c3c2=O)c(F)n1 ; O=c1c2c(F)cnn2nc(SCc2ccc(C(F)(F)F)cc2)n1-c1ncc(O)nc1F]

Pyridinium hydrochloride (36.0 mg, 62.0 μmol, 5.00 equiv) is added to 5-fluoro-3-(3-fluoro-5-methoxypyrazin-2-yl)-2-((4-(trifluoromethyl)benzyl)thio)pyrazolo[5,1-f][1,2,4]-triazin-4(3H)-one (intermediate 59, 73.0 mg, 62.0 μmol, 1 equiv) and the mixture is heated at 250° C. for 2 minutes with a heatgun. The mixture is purified by reversed phase HPLC (Agilent Sunfire™-C18, gradient of acetonitrile in water, 0.1% TFA) to yield the desired product.

| | |
|---|---|
| C₁₇H₉F₅N₆O₂S | (MW = 456.0 g/mol) |
| ESI | 457 [M + H]⁺ |
| Rₜ (HPLC) | 0.98 min (Z018_S04) |

### Example 27

### 3-(5-Hydroxypyrazin-2-yl)-2-((4-(trifluoromethyl)benzyl)thio)pyrazolo[5,1-f][1,2,4]triazin-4(3H)-one

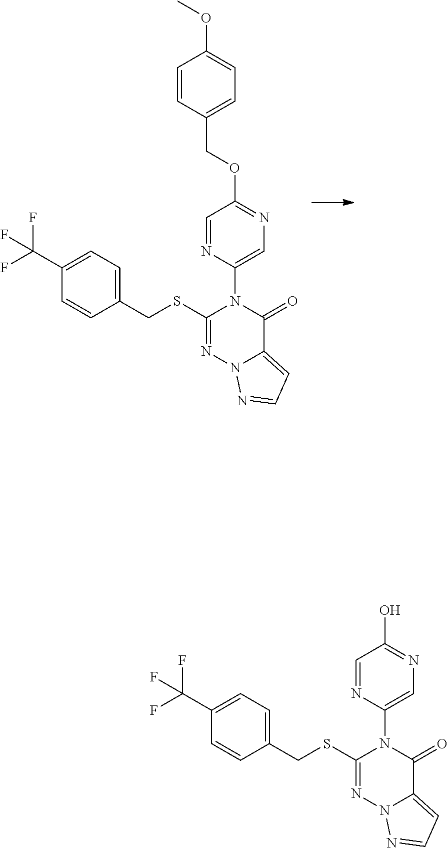 [SMILES: COc1ccc(COc2cnc(-n3c(SCc4ccc(C(F)(F)F)cc4)nn4nccc4c3=O)cn2)cc1 ; O=c1c2ccnn2nc(SCc2ccc(C(F)(F)F)cc2)n1-c1cnc(O)cn1]

A mixture of 2-((2,4-difluorobenzyl)thio)-3-(5-((4-methoxybenzyl)oxy)pyrazin-2-yl)pyrazolo[5,1-f][1,2,4]triazin-4(3H)-one (intermediate 59, 270 mg, 500 μmol, 1 equiv) in acetic acid (2.00 mL) is heated at 120° C. After 2 h, the reaction mixture is concentrated under reduced pressure and the residue is purified by reversed phase HPLC (Agilent Sunfire™-C18, gradient of acetonitrile in water, 0.1% TFA) to yield the desired product.

| | |
|---|---|
| C₁₇H₁₁F₃N₆O₂S | (MW = 420.1 g/mol) |
| ESI | 421 [M + H]⁺ |
| Rₜ (HPLC) | 0.90 min (Z018_S04) |

### Example 28

### 2-((2-Fluoro-4-(trifluoromethyl)benzyl)thio)-3-(5-hydroxypyrazin-2-yl)pyrazolo[5,1-f][1,2,4]triazin-4(3H)-one

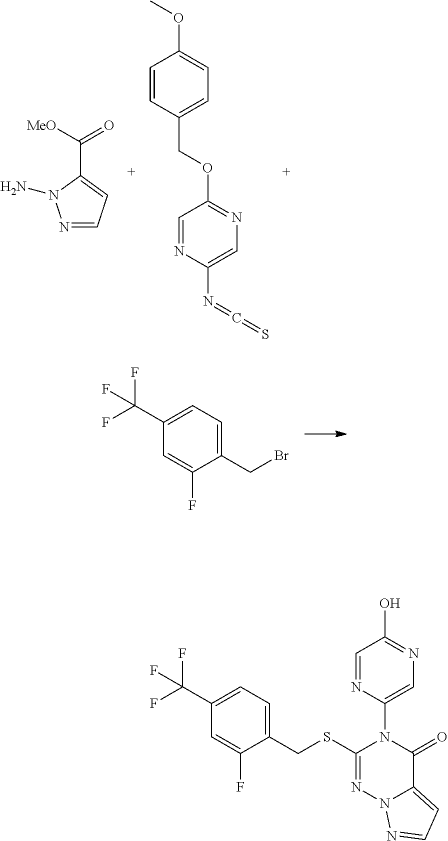 [SMILES: COC(=O)c1ccnn1N ; COc1ccc(COc2cnc(N=C=S)cn2)cc1 ; Fc1cc(C(F)(F)F)ccc1CBr ; O=c1c2ccnn2nc(SCc2ccc(C(F)(F)F)cc2F)n1-c1cnc(O)cn1]

2-Isothiocyanato-5-((4-methoxybenzyl)oxy)pyrazine (intermediate 25, 1.19 g, 4.25 mmol, 1 equiv) is added to a solution of methyl 1-amino-1H-pyrazole-5-carboxylate (intermediate 2, 600 mg, 4.25 mmol, 1 equiv) in acetonitrile (20 mL). After 18 h, potassium tert-butoxide (460 mg, 4.25 mmol, 1 equiv) is added and the reaction mixture is heated at 50° C. After 2 h, 1-(bromomethyl)-2-fluoro-4-(trifluoromethyl)benzene (1.13 g, 4.18 mmol, 1 equiv) is added. After 2 h, water and ethyl acetate are added, the layers are separated, and the organic layer is concentrated under reduced pressure. The residue is dissolved in acetic acid (5.00 mL) and the mixture is heated at 120° C. After 2 h, the reaction mixture is concentrated under reduced pressure and the residue is purified by reversed phase HPLC (Gilson Sunfire™-C18, gradient of acetonitrile in water, 0.1% TFA) to yield the desired product.

| | |
|---|---|
| C₁₇H₁₀F₄N₆O₂S | (MW = 438.1 g/mol) |
| ESI | 439 [M + H]⁺ |
| Rₜ (HPLC) | 0.90 min (Z018_S04) |

The following examples are prepared using procedures analogous to those described for example 28, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 29 | 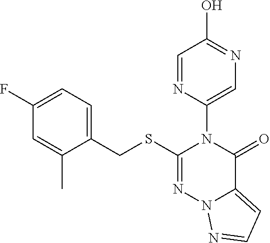 [SMILES: Cc1cc(F)ccc1CSc1nn2nccc2c(=O)n1-c1cnc(O)cn1] | 385 [M + H]⁺ | 0.84 (Z018_S04) |
| 30 | 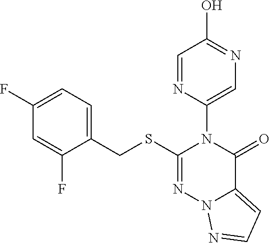 [SMILES: O=c1c2ccnn2nc(SCc2ccc(F)cc2F)n1-c1cnc(O)cn1] | 389 [M + H]⁺ | 0.83 (Z018_S04) |
| 31 | 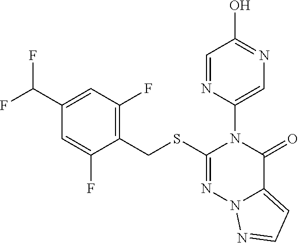 [SMILES: O=c1c2ccnn2nc(SCc2c(F)cc(C(F)F)cc2F)n1-c1cnc(O)cn1] | 439 [M + H]⁺ | 0.85 (Z018_S04) |
| 32 | 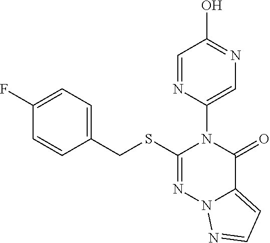 [SMILES: O=c1c2ccnn2nc(SCc2ccc(F)cc2)n1-c1cnc(O)cn1] | 371 [M + H]⁺ | 0.80 (Z018_S04) |
| 33 | 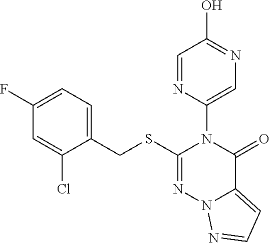 [SMILES: O=c1c2ccnn2nc(SCc2ccc(F)cc2Cl)n1-c1cnc(O)cn1] | 405/407 [M + H]⁺ | 0.85 (Z018_S04) |
| 34 | 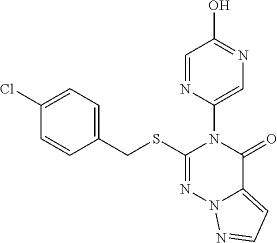 [SMILES: O=c1c2ccnn2nc(SCc2ccc(Cl)cc2)n1-c1cnc(O)cn1] | 387/389 [M + H]⁺ | 0.87 (Z018_S04) |
| 35 | 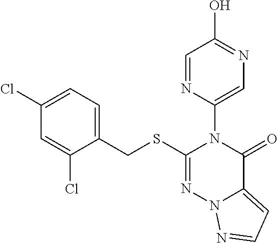 [SMILES: O=c1c2ccnn2nc(SCc2ccc(Cl)cc2Cl)n1-c1cnc(O)cn1] | 421/423 [M + H]⁺ | 0.90 (Z018_S04) |
| 36 | 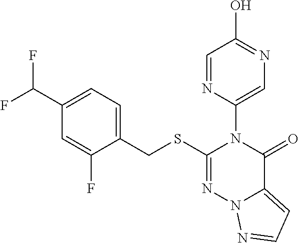 [SMILES: O=c1c2ccnn2nc(SCc2ccc(C(F)F)cc2F)n1-c1cnc(O)cn1] | 421 [M + H]⁺ | 0.85 (Z018_S04) |
| 37 | 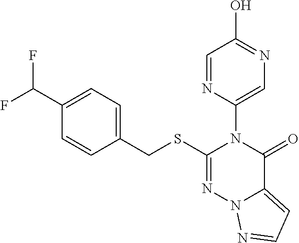 [SMILES: O=c1c2ccnn2nc(SCc2ccc(C(F)F)cc2)n1-c1cnc(O)cn1] | 403 [M + H]⁺ | 0.83 (Z018_S04) |
| 38 | 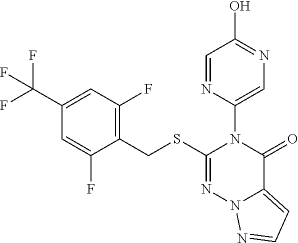 [SMILES: O=c1c2ccnn2nc(SCc2c(F)cc(C(F)(F)F)cc2F)n1-c1cnc(O)cn1] | 457 [M + H]⁺ | 0.90 (Z018_S04) |
| 39 | 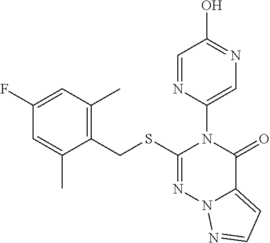 [SMILES: Cc1cc(F)cc(C)c1CSc1nn2nccc2c(=O)n1-c1cnc(O)cn1] | 399 [M + H]⁺ | 0.88 (Z018_S04) |
| 40 | 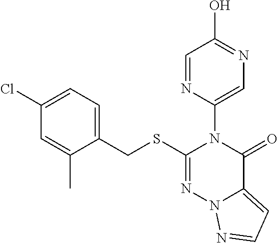 [SMILES: Cc1cc(Cl)ccc1CSc1nn2nccc2c(=O)n1-c1cnc(O)cn1] | 401/403 [M + H]⁺ | 0.89 (Z018_S04) |
| 41 | 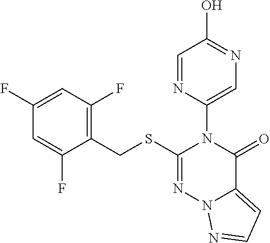 [SMILES: O=c1c2ccnn2nc(SCc2c(F)cc(F)cc2F)n1-c1cnc(O)cn1] | 407 [M + H]⁺ | 0.83 (Z018_S04) |
| 42 | 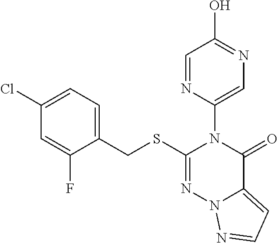 [SMILES: O=c1c2ccnn2nc(SCc2ccc(Cl)cc2F)n1-c1cnc(O)cn1] | 405/407 [M + H]⁺ | 0.86 (Z018_S04) |
| 43 | 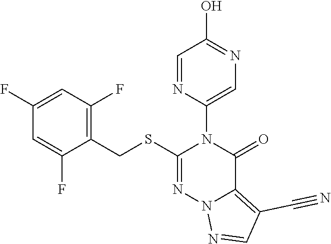 [SMILES: N#Cc1cnn2nc(SCc3c(F)cc(F)cc3F)n(-c3cnc(O)cn3)c(=O)c12] | 432 [M + H]⁺ | 0.87 (Z018_S04) |
| 44 | 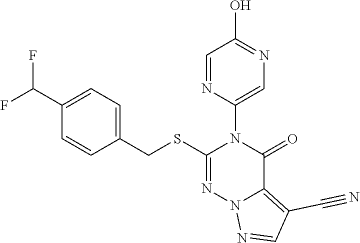 [SMILES: N#Cc1cnn2nc(SCc3ccc(C(F)F)cc3)n(-c3cnc(O)cn3)c(=O)c12] | 428 [M + H]⁺ | 0.87 (Z018_S04) |

### Example 45

### 2-((4-Chlorobenzyl)thio)-5-fluoro-3-(5-hydroxypyrazin-2-yl)pyrazolo[5,1-f][1,2,4]triazin-4(3H)-on

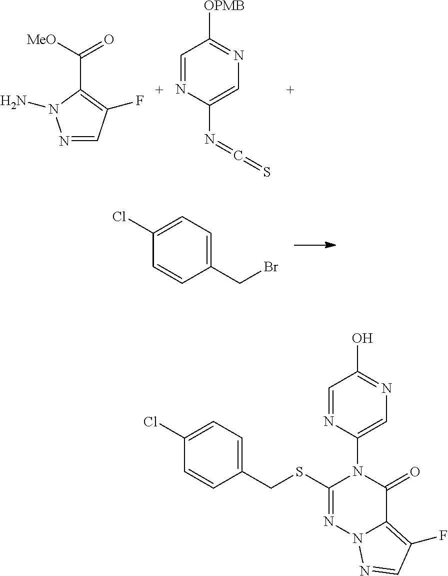

2-Isothiocyanato-5-((4-methoxybenzyl)oxy)pyrazine (intermediate 1, 1.54 g, 5.53 mmol, 1 equiv) is added to a solution of methyl 1-amino-4-fluoro-1H-pyrazole-5-carboxylate (intermediate 25, 880 mg, 5.53 mmol, 1 equiv) in acetonitrile (18 mL). After 72 h, potassium tert-butoxide (620 mg, 5.53 mmol, 1 equiv) is added. After 1 h, 1-(bromomethyl)-4-chlorobenzene (1.16 g, 5.53 mmol, 1 equiv) is added. After 30 min, water and ethyl acetate are added, the layers are separated, and the organic layer is concentrated under reduced pressure. The residue is dissolved in methanol (52 mL), Amberlyst® 15(H) (2.16 g, 9.94 mmol, 1.80 equiv) is added and the reaction mixture is heated at reflux. After 3 h, the reaction mixture is filtered, and the filtrate is concentrated under reduced pressure. The residue is washed with dichloromethane and dried to yield the product.

| | |
|---|---|
| C₁₆H₁₀ClFN₆O₂S | (MW = 404.0 g/mol) |
| ESI | 405/407 [M + H]⁺ |
| Rₜ (HPLC) | 0.92 min (Z018_S04) |

The following compounds are prepared using procedures analogous to those described for example 45, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 46 | 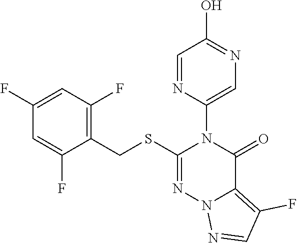 [SMILES: O=c1c2c(F)cnn2nc(SCc2c(F)cc(F)cc2F)n1-c1cnc(O)cn1] | 425 [M + H]⁺ | 0.87 (Z018_S04) |

### Example 47

### 5-Fluoro-3-(5-hydroxypyrazin-2-yl)-2-((4-(trifluoromethyl)benzyl)thio)pyrazolo[5,1-f][1,2,4]triazin-4(3H)-one

### Step 1

### Methyl 4-fluoro-1-(3-(5-((4-methoxybenzyl)oxy)pyrazin-2-yl)thioureido)-1H-pyrazole-5-carboxylate

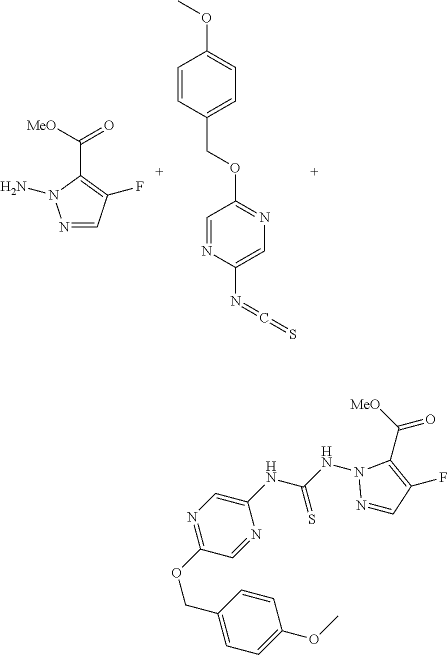 [SMILES: COC(=O)c1c(F)cnn1N ; COc1ccc(COc2cnc(N=C=S)cn2)cc1 ; COC(=O)c1c(F)cnn1NC(=S)Nc1cnc(OCc2ccc(OC)cc2)cn1]

2-Isothiocyanato-5-((4-methoxybenzyl)oxy)pyrazine (intermediate 1, 982 mg, 3.52 mmol, 1 equiv) is added to a solution of methyl 1-amino-4-fluoro-1H-pyrazole-5-carboxylate (intermediate 25, 560 mg, 3.52 mmol, 1 equiv) in acetonitrile (10 mL). The mixture is stirred at room temperature over night and concentrated under reduced pressure. An aliquot (approximately ⅘≈80%) of the residue is taken into the next step without further purification.

### Step 2

### 5-Fluoro-3-(5-((4-methoxybenzyl)oxy)pyrazin-2-yl)-2-thioxo-2,3-dihydropyrazolo[5,1-f][1,2,4]triazin-4(1H)-one

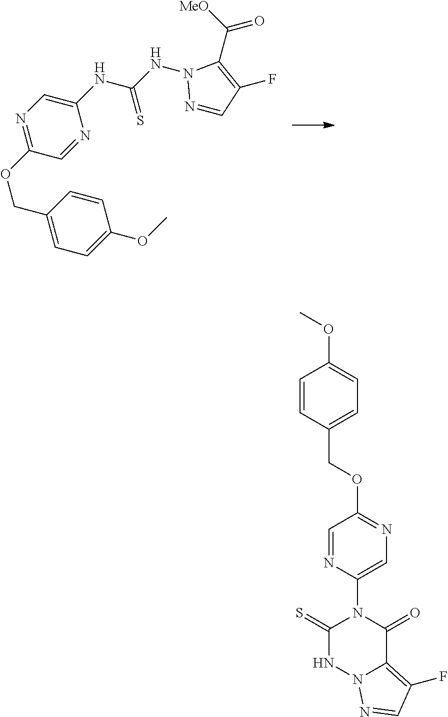 [SMILES: COC(=O)c1c(F)cnn1NC(=S)Nc1cnc(OCc2ccc(OC)cc2)cn1 ; COc1ccc(COc2cnc(-n3c(=S)[nH]n4ncc(F)c4c3=O)cn2)cc1]

An aliquot (approximately ⅘≈80%) from the residue from step 1 is dissolved in N,N-dimethylacetamide (8 mL), and the mixture is treated with potassium tert-butoxide (311 mg, 2.78 μmol, 1.00 equiv) and heated at 50° C. for 3 h. The mixture is cooled at room temperature and split, and an aliquot (¼≈25%) is taken directly into the next step.

### Step 3

### 5-Fluoro-3-(5-hydroxypyrazin-2-yl)-2-((4-(trifluoromethyl)benzyl)thio)pyrazolo[5,1-f][1,2,4]triazin-4(3H)-one

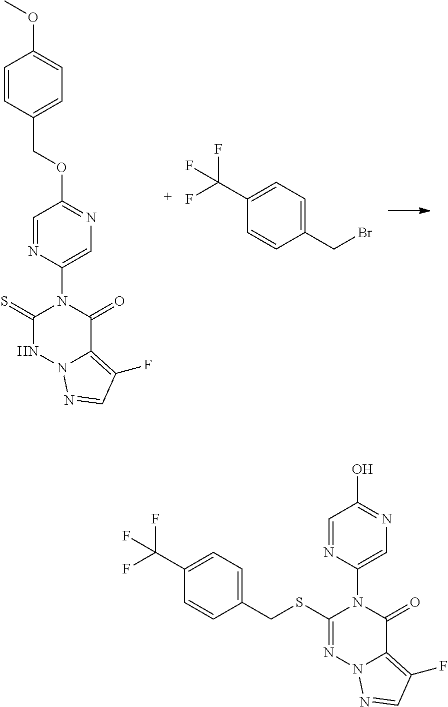 [SMILES: COc1ccc(COc2cnc(-n3c(=S)[nH]n4ncc(F)c4c3=O)cn2)cc1 ; FC(F)(F)c1ccc(CBr)cc1 ; O=c1c2c(F)cnn2nc(SCc2ccc(C(F)(F)F)cc2)n1-c1cnc(O)cn1]

An aliquot (¼≈25%) from the mixture of step 2 is treated with N,N-Diisopropylethylamine (356 μL, 2.1 mmol, 3.0 equiv) and 4-(trifluormethyl)benzyl bromide (167 mg, 685 μmol, 1.00 equiv), and the mixture is stirred at room temperature for 30 min. Water (20 mL) and ethyl acetate (20 mL) is added, and the layers are separated. The organic layer is washed with water and concentrated under reduced pressure. The residue is dissolved in methanol (6.8 mL), and treated with Amberlyst® 15(H) (266 mg, 1.22 mmol, 1.8 equiv). The mixture is heated at reflux for 1.5 h and filtered. The filtrate is concentrated under reduced pressure, and the residue is purified by reversed phase HPLC (Agilent Sunfire™-C18, gradient of acetonitrile in water, 0.1% TFA) to yield the desired product.

| | |
|---|---|
| C₁₇H₁₀F₄N₆O₂S | (MW = 438.4 g/mol) |
| ESI | 439 [M + H]⁺ |
| Rₜ (HPLC) | 0.92 min (Z018_S04) |

### Example 48

### 5-Fluoro-3-(5-hydroxypyrazin-2-yl)-2-((4-(difluoromethyl)benzyl)thio)pyrazolo[5,1-f][1,2,4]triazin-4(3H)-one

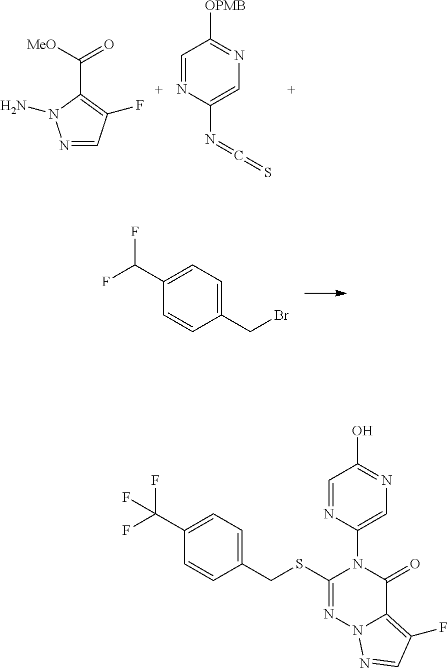

2-Isothiocyanato-5-((4-methoxybenzyl)oxy)pyrazine (intermediate 1, 112 mg, 0.40 mmol, 1 equiv) is added to a solution of methyl 1-amino-4-fluoro-1H-pyrazole-5-carboxylate (intermediate 25, 65 mg, 0.40 mmol, 1 equiv) in acetonitrile (3 mL), and the mixture is stirred at room temperature for 24 h. Potassium tert-butoxide (45 mg, 0.40 mmol, 1 equiv) is added, and the mixture is stirred at room temperature for 1 h. N,N-Diisopropylethylamine (220 μL, 1.27 mmol, 3 equiv), N,N-dimethylacetamide (1.5 mL) and 4-(difluoromethyl)benzyl bromide (110 mg, 0.47 mmol, 1.1 equiv) is added, and the mixture is stirred at room temperature for 1 h. All volatiles are removed under reduced pressure, and the residue is extracted with dichloromethane and water. The organic layer is separated and concentrated under reduced pressure. The residue is dissolved in MeOH (2 mL), and the mixture is treated with Amberlyst® 15(H) (200 mg, 0.92 mmol, 2.2 equiv), heated at reflux for 3 h and filtered. The residue is purified by reversed phase HPLC (Agilent Sunfire™-C18, gradient of acetonitrile in water, 0.1% TFA) to yield the desired product.

| | |
|---|---|
| C₁₇H₁₁F₃N₆O₂S | (MW = 420.4 g/mol) |
| ESI | 421 [M + H]⁺ |
| Rₜ (HPLC) | 0.89 min (Z018_S04) |

The following compounds are prepared using procedures analogous to those described for example 48, using appropriate starting materials. As is appreciated by those skilled in the art, the analogous examples may involve variations in general reaction conditions.

| Ex- | | | HPLC retention time |
|---|---|---|---|
| ample | Structure | ESI-MS | [min] (method) |
| 49 | 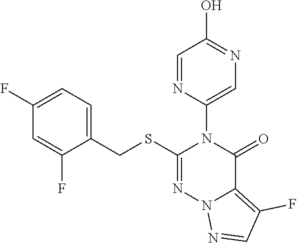 [SMILES: O=c1c2c(F)cnn2nc(SCc2ccc(F)cc2F)n1-c1cnc(O)cn1] | 407 [M + H]⁺ | 0.86 (Z018_S04) |
| 50 | 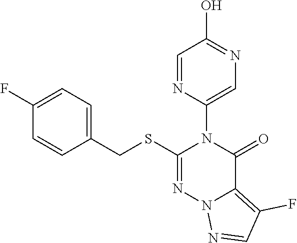 [SMILES: O=c1c2c(F)cnn2nc(SCc2ccc(F)cc2)n1-c1cnc(O)cn1] | 389 [M + H]⁺ | 0.88 (Z018_S04) |
| 51 | 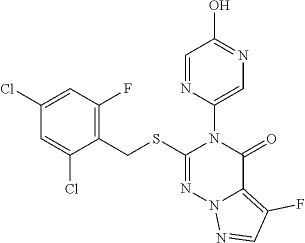 [SMILES: O=c1c2c(F)cnn2nc(SCc2c(F)cc(Cl)cc2Cl)n1-c1cnc(O)cn1] | 457/459 [M + H]⁺ | 0.97 (Z018_S04) |
| 52 | 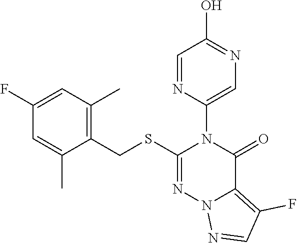 [SMILES: Cc1cc(F)cc(C)c1CSc1nn2ncc(F)c2c(=O)n1-c1cnc(O)cn1] | 417 [M + H]⁺ | 0.95 (Z018_S04) |
| 53 | 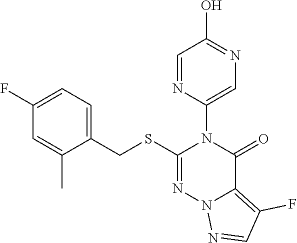 [SMILES: Cc1cc(F)ccc1CSc1nn2ncc(F)c2c(=O)n1-c1cnc(O)cn1] | 403 [M + H]⁺ | 0.91 (Z018_S04) |
| 54 | 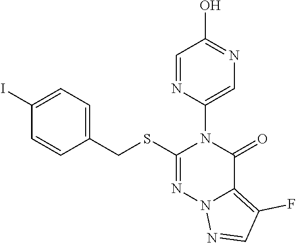 [SMILES: O=c1c2c(F)cnn2nc(SCc2ccc(I)cc2)n1-c1cnc(O)cn1] | 497 [M + H]⁺ | 0.95 (Z018_S04) |
| 55 | 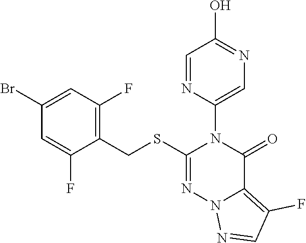 [SMILES: O=c1c2c(F)cnn2nc(SCc2c(F)cc(Br)cc2F)n1-c1cnc(O)cn1] | 485/487 [M + H]⁺ | 0.94 (Z018_S04) |
| 56 | 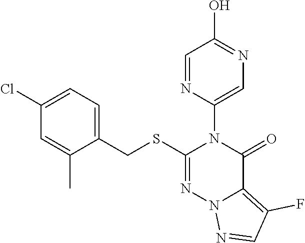 [SMILES: Cc1cc(Cl)ccc1CSc1nn2ncc(F)c2c(=O)n1-c1cnc(O)cn1] | 419/421 [M + H]⁺ | 0.96 (Z018_S04) |
| 57 | 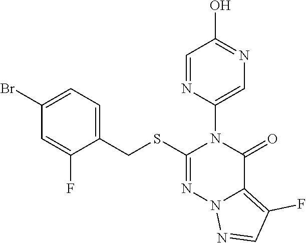 [SMILES: O=c1c2c(F)cnn2nc(SCc2ccc(Br)cc2F)n1-c1cnc(O)cn1] | 467/469 [M + H]⁺ | 0.94 (Z018_S04) |
| 58 | 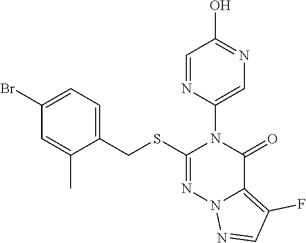 [SMILES: Cc1cc(Br)ccc1CSc1nn2ncc(F)c2c(=O)n1-c1cnc(O)cn1] | 463/465 [M + H]⁺ | 0.97 (Z018_S04) |
| 59 | 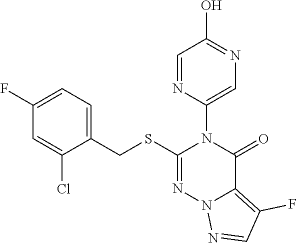 [SMILES: O=c1c2c(F)cnn2nc(SCc2ccc(F)cc2Cl)n1-c1cnc(O)cn1] | 423/425 [M + H]⁺ | 0.92 (Z018_S04) |
| 60 | 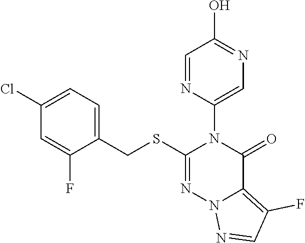 [SMILES: O=c1c2c(F)cnn2nc(SCc2ccc(Cl)cc2F)n1-c1cnc(O)cn1] | 423/425 [M + H]⁺ | 0.93 (Z018_S04) |
| 61 | 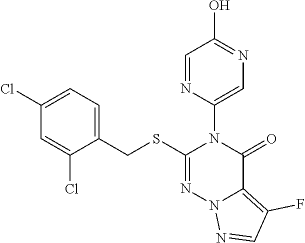 [SMILES: O=c1c2c(F)cnn2nc(SCc2ccc(Cl)cc2Cl)n1-c1cnc(O)cn1] | 439/441 [M + H]⁺ | 0.97 (Z018_S04) |
| 62 | 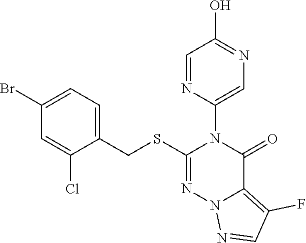 [SMILES: O=c1c2c(F)cnn2nc(SCc2ccc(Br)cc2Cl)n1-c1cnc(O)cn1] | 483/485 [M + H]⁺ | 0.98 (Z018_S04) |
| 63 | 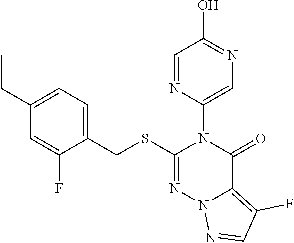 [SMILES: CCc1ccc(CSc2nn3ncc(F)c3c(=O)n2-c2cnc(O)cn2)c(F)c1] | 417 [M + H]⁺ | 0.96 (Z018_S04) |
| 64 | 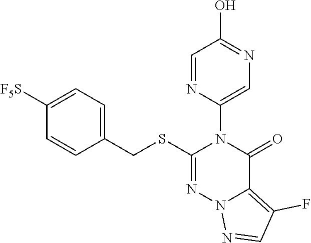 | 497 [M + H]⁺ | 0.97 (Z018_S04) |
| 65 | 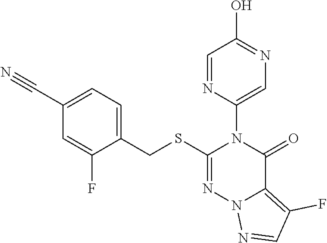 [SMILES: N#Cc1ccc(CSc2nn3ncc(F)c3c(=O)n2-c2cnc(O)cn2)c(F)c1] | 414 [M + H]⁺ | 0.84 (Z018_S04) |
| 66 | 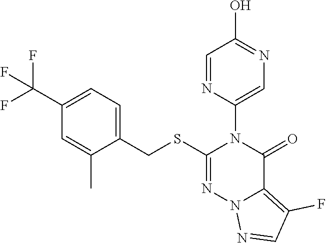 [SMILES: Cc1cc(C(F)(F)F)ccc1CSc1nn2ncc(F)c2c(=O)n1-c1cnc(O)cn1] | 453 [M + H]⁺ | 0.98 (Z018_S04) |
| 67 | 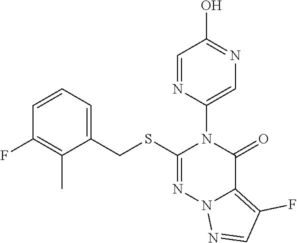 [SMILES: Cc1c(F)cccc1CSc1nn2ncc(F)c2c(=O)n1-c1cnc(O)cn1] | 403 [M + H]⁺ | 0.91 (Z018_S04) |
| 68 | 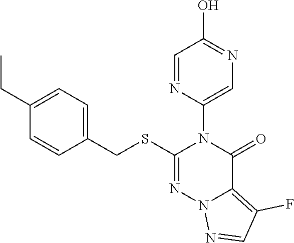 [SMILES: CCc1ccc(CSc2nn3ncc(F)c3c(=O)n2-c2cnc(O)cn2)cc1] | 399 [M + H]⁺ | 0.95 (Z018_S04) |
| 69 | 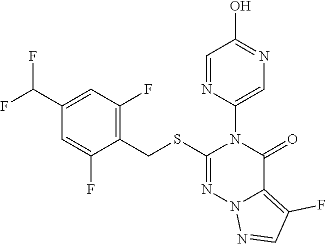 [SMILES: O=c1c2c(F)cnn2nc(SCc2c(F)cc(C(F)F)cc2F)n1-c1cnc(O)cn1] | 457 [M + H]⁺ | 0.90 (Z018_S04) |
| 70 | 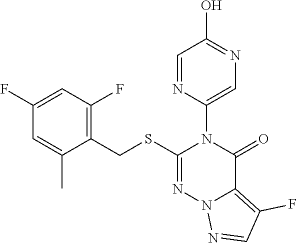 [SMILES: Cc1cc(F)cc(F)c1CSc1nn2ncc(F)c2c(=O)n1-c1cnc(O)cn1] | 421 [M + H]⁺ | 0.92 (Z018_S04) |
| 71 | 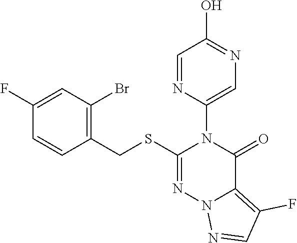 [SMILES: O=c1c2c(F)cnn2nc(SCc2ccc(F)cc2Br)n1-c1cnc(O)cn1] | 467/469 [M + H]⁺ | 0.93 (Z018_S04) |
| 72 | 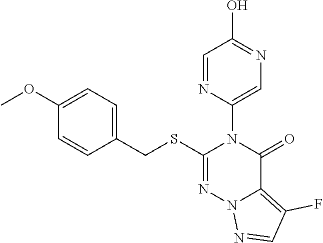 [SMILES: COc1ccc(CSc2nn3ncc(F)c3c(=O)n2-c2cnc(O)cn2)cc1] | 401 [M + H]⁺ | 0.86 (Z018_S04) |
| 73 | 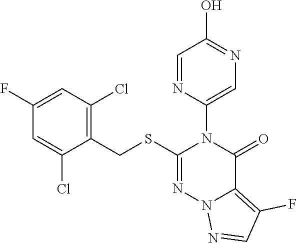 [SMILES: O=c1c2c(F)cnn2nc(SCc2c(Cl)cc(F)cc2Cl)n1-c1cnc(O)cn1] | 457/459 [M + H]⁺ | 0.96 (Z018_S04) |
| 74 | 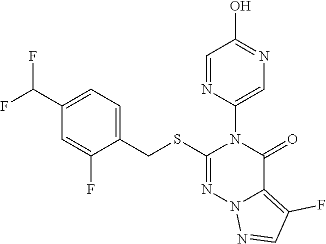 [SMILES: O=c1c2c(F)cnn2nc(SCc2ccc(C(F)F)cc2F)n1-c1cnc(O)cn1] | 439 [M + H]⁺ | 0.89 (Z018_S04) |
| 75 | 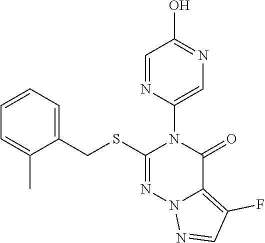 [SMILES: Cc1ccccc1CSc1nn2ncc(F)c2c(=O)n1-c1cnc(O)cn1] | 385 [M + H]⁺ | 0.90 (Z018_S04) |
| 76 | 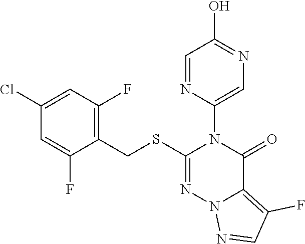 [SMILES: O=c1c2c(F)cnn2nc(SCc2c(F)cc(Cl)cc2F)n1-c1cnc(O)cn1] | 441/443 [M + H]⁺ | 0.93 (Z018_S04) |
| 77 | 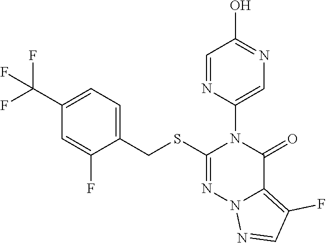 [SMILES: O=c1c2c(F)cnn2nc(SCc2ccc(C(F)(F)F)cc2F)n1-c1cnc(O)cn1] | 457 [M + H]⁺ | 0.95 (Z018_S04) |
| 78 | 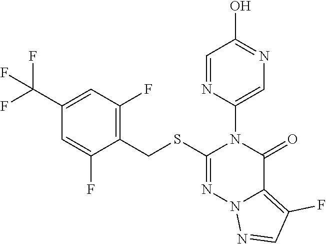 [SMILES: O=c1c2c(F)cnn2nc(SCc2c(F)cc(C(F)(F)F)cc2F)n1-c1cnc(O)cn1] | 475 [M + H]⁺ | 0.95 (Z018_S04) |
| 79 | 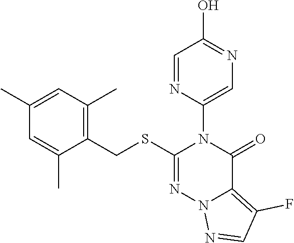 [SMILES: Cc1cc(C)c(CSc2nn3ncc(F)c3c(=O)n2-c2cnc(O)cn2)c(C)c1] | 413 [M + H]⁺ | 0.98 (Z018_S04) |
| 80 | 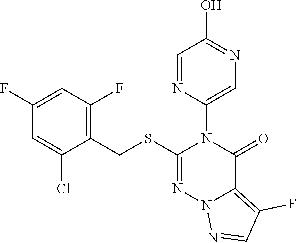 [SMILES: O=c1c2c(F)cnn2nc(SCc2c(F)cc(F)cc2Cl)n1-c1cnc(O)cn1] | 441/443 [M + H]⁺ | 0.92 (Z018_S04) |
| 81 | 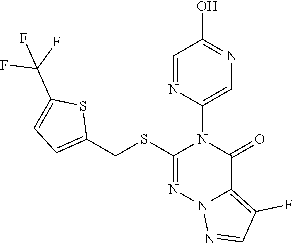 [SMILES: O=c1c2c(F)cnn2nc(SCc2ccc(C(F)(F)F)s2)n1-c1cnc(O)cn1] | 445 [M + H]⁺ | 0.94 (Z018_S04) |
| 82 | 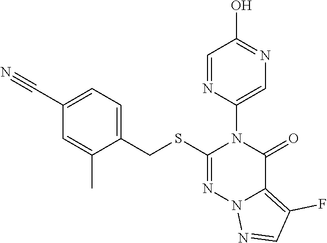 [SMILES: Cc1cc(C#N)ccc1CSc1nn2ncc(F)c2c(=O)n1-c1cnc(O)cn1] | 410 [M + H]⁺ | 0.96 (Z018_S04) |
| 83 | 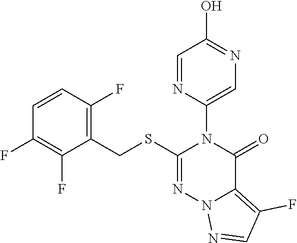 [SMILES: O=c1c2c(F)cnn2nc(SCc2c(F)ccc(F)c2F)n1-c1cnc(O)cn1] | 425 [M + H]⁺ | 0.88 (Z018_S04) |
| 84 | 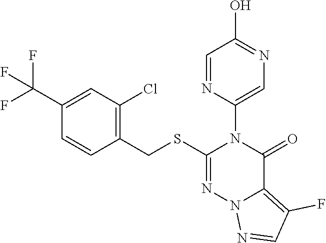 [SMILES: O=c1c2c(F)cnn2nc(SCc2ccc(C(F)(F)F)cc2Cl)n1-c1cnc(O)cn1] | 473/475 [M + H]⁺ | 0.99 (Z018_S04) |
| 85 | 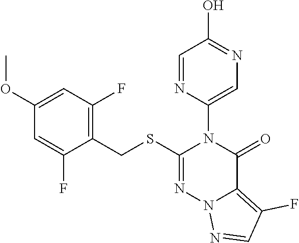 [SMILES: COc1cc(F)c(CSc2nn3ncc(F)c3c(=O)n2-c2cnc(O)cn2)c(F)c1] | 437 [M + H]⁺ | 0.90 (Z018_S04) |
| 86 | 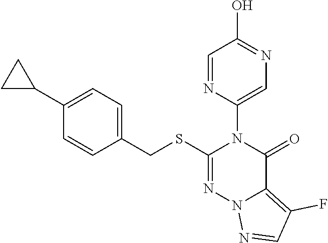 [SMILES: O=c1c2c(F)cnn2nc(SCc2ccc(C3CC3)cc2)n1-c1cnc(O)cn1] | 411 [M + H]⁺ | 0.95 (Z018_S04) |
| 87 | 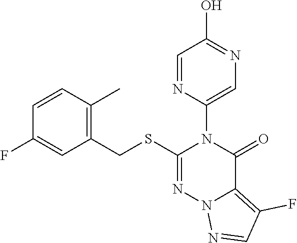 [SMILES: Cc1ccc(F)cc1CSc1nn2ncc(F)c2c(=O)n1-c1cnc(O)cn1] | 403 [M + H]⁺ | 0.91 (Z018_S04) |
| 88 | 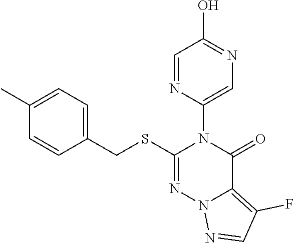 [SMILES: Cc1ccc(CSc2nn3ncc(F)c3c(=O)n2-c2cnc(O)cn2)cc1] | 385 [M + H]⁺ | 0.91 (Z018_S04) |
| 89 | 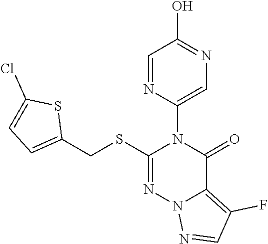 [SMILES: O=c1c2c(F)cnn2nc(SCc2ccc(Cl)s2)n1-c1cnc(O)cn1] | 411/413 [M + H]⁺ | 0.91 (Z018_S04) |
| 90 | 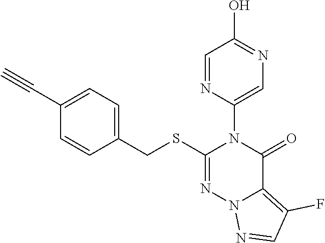 [SMILES: C#Cc1ccc(CSc2nn3ncc(F)c3c(=O)n2-c2cnc(O)cn2)cc1] | 395 [M + H]⁺ | 0.88 (Z018_S04) |
| 91 | 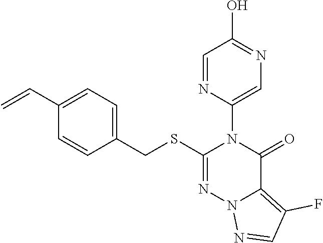 [SMILES: C=Cc1ccc(CSc2nn3ncc(F)c3c(=O)n2-c2cnc(O)cn2)cc1] | 397 [M + H]⁺ | 0.92 (Z018_S04) |
| 92 | 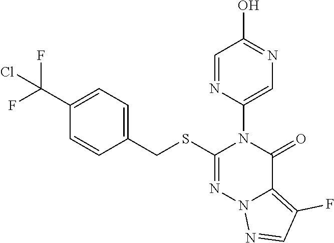 [SMILES: O=c1c2c(F)cnn2nc(SCc2ccc(C(F)(F)Cl)cc2)n1-c1cnc(O)cn1] | 455 [M + H]⁺ | 0.97 (Z018_S04) |
| 93 | 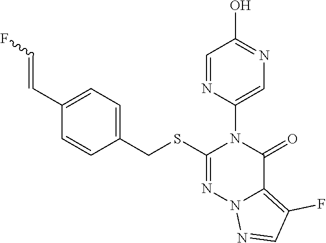 [SMILES: O=c1c2c(F)cnn2nc(SCc2ccc(C=CF)cc2)n1-c1cnc(O)cn1] | 415 [M + H]⁺ | 0.90 (Z018_S04) |
| 94 | 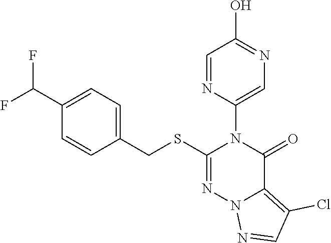 [SMILES: O=c1c2c(Cl)cnn2nc(SCc2ccc(C(F)F)cc2)n1-c1cnc(O)cn1] | 435 [M − H]⁻ | 0.90 (Z018_S04) |
| 95 | 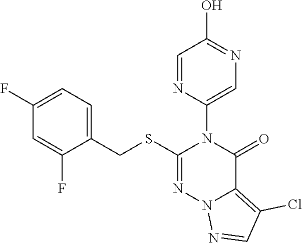 [SMILES: O=c1c2c(Cl)cnn2nc(SCc2ccc(F)cc2F)n1-c1cnc(O)cn1] | 423/425 [M + H]⁺ | 0.91 (Z018_S04) |
| 96 | 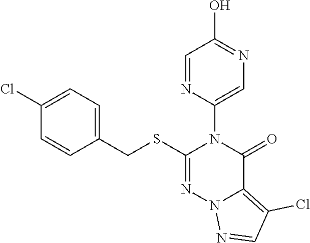 [SMILES: O=c1c2c(Cl)cnn2nc(SCc2ccc(Cl)cc2)n1-c1cnc(O)cn1] | 421/423 [M + H]⁺ | 0.93 (Z018_S04) |
| 97 | 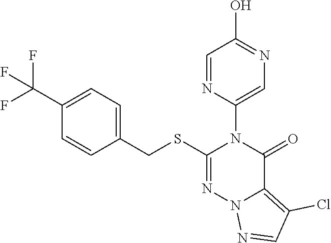 [SMILES: O=c1c2c(Cl)cnn2nc(SCc2ccc(C(F)(F)F)cc2)n1-c1cnc(O)cn1] | 455/457 [M + H]⁺ | 0.96 (Z018_S04) |
| 98 | 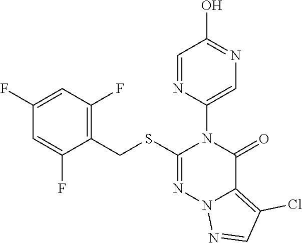 [SMILES: O=c1c2c(Cl)cnn2nc(SCc2c(F)cc(F)cc2F)n1-c1cnc(O)cn1] | 441/443 [M + H]⁺ | 0.90 (Z018_S04) |
| 99 | 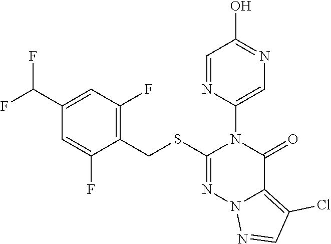 [SMILES: O=c1c2c(Cl)cnn2nc(SCc2c(F)cc(C(F)F)cc2F)n1-c1cnc(O)cn1] | 473/475 [M + H]⁺ | 0.92 (Z018_S04) |
| 100 | 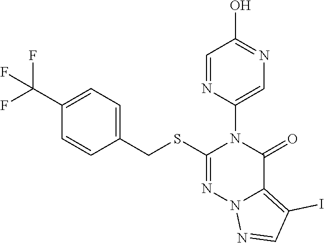 [SMILES: O=c1c2c(I)cnn2nc(SCc2ccc(C(F)(F)F)cc2)n1-c1cnc(O)cn1] | 547 [M + H]⁺ | 0.62 (X012_S01) |
| 101 | 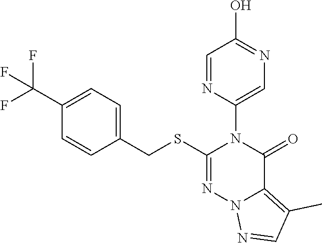 [SMILES: Cc1cnn2nc(SCc3ccc(C(F)(F)F)cc3)n(-c3cnc(O)cn3)c(=O)c12] | 435 [M + H]⁺ | 0.58 (X012_S01) |
| 102 | 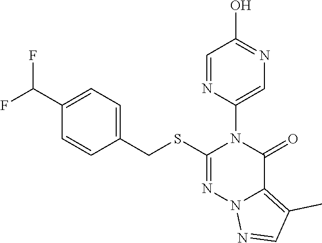 [SMILES: Cc1cnn2nc(SCc3ccc(C(F)F)cc3)n(-c3cnc(O)cn3)c(=O)c12] | 417 [M + H]⁺ | 0.52 (X012_S01) |
| 103 | 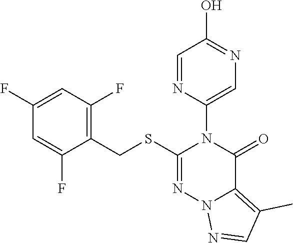 [SMILES: Cc1cnn2nc(SCc3c(F)cc(F)cc3F)n(-c3cnc(O)cn3)c(=O)c12] | 421 [M + H]⁺ | 0.53 (X012_S01) |
| 104 | 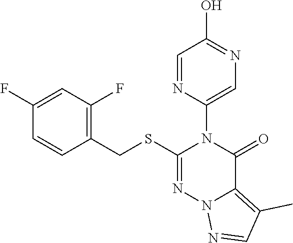 [SMILES: Cc1cnn2nc(SCc3ccc(F)cc3F)n(-c3cnc(O)cn3)c(=O)c12] | 403 [M + H]⁺ | 0.52 (X012_S01) |
| 105 | 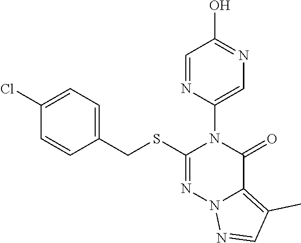 [SMILES: Cc1cnn2nc(SCc3ccc(Cl)cc3)n(-c3cnc(O)cn3)c(=O)c12] | 401/403 [M + H]⁺ | 0.55 (X012_S01) |
| 106 | 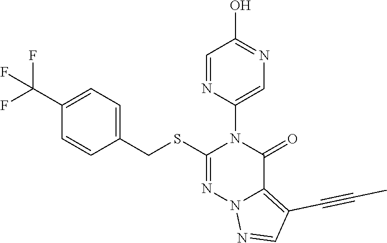 [SMILES: CC#Cc1cnn2nc(SCc3ccc(C(F)(F)F)cc3)n(-c3cnc(O)cn3)c(=O)c12] | 459 [M + H]⁺ | 0.88 (X018_S01) |
| 107 | 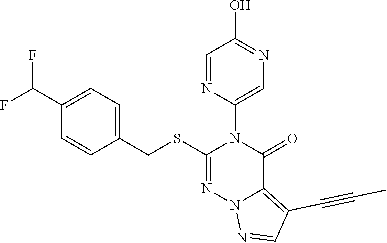 [SMILES: CC#Cc1cnn2nc(SCc3ccc(C(F)F)cc3)n(-c3cnc(O)cn3)c(=O)c12] | 441 [M + H]⁺ | 0.81 (X018_S03) |
| 108 | 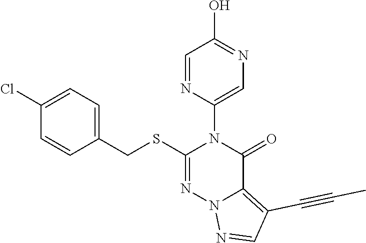 [SMILES: CC#Cc1cnn2nc(SCc3ccc(Cl)cc3)n(-c3cnc(O)cn3)c(=O)c12] | 425 [M + H]⁺ | 0.85 (X018_S03) |
| 109 | 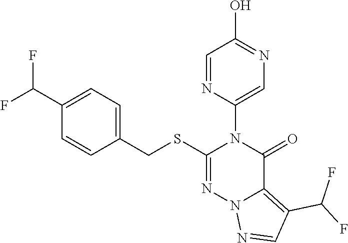 [SMILES: O=c1c2c(C(F)F)cnn2nc(SCc2ccc(C(F)F)cc2)n1-c1cnc(O)cn1] | 453 [M + H]⁺ | 0.81 (X018_S03) |
| 110 | 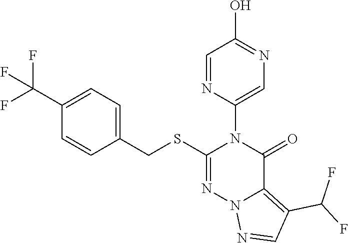 [SMILES: O=c1c2c(C(F)F)cnn2nc(SCc2ccc(C(F)(F)F)cc2)n1-c1cnc(O)cn1] | 471 [M + H]⁺ | 0.89 (X018_S03) |
| 111 | 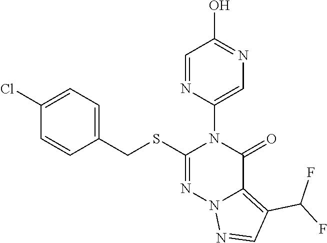 [SMILES: O=c1c2c(C(F)F)cnn2nc(SCc2ccc(Cl)cc2)n1-c1cnc(O)cn1] | 437 [M + H]⁺ | 0.85 (X018_S03) |
| 112 | 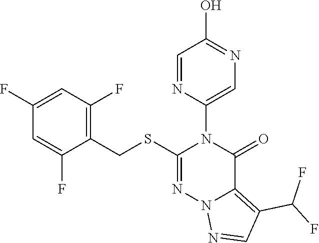 [SMILES: O=c1c2c(C(F)F)cnn2nc(SCc2c(F)cc(F)cc2F)n1-c1cnc(O)cn1] | 457 [M + H]⁺ | 0.82 (X018_S03) |
| 113 | 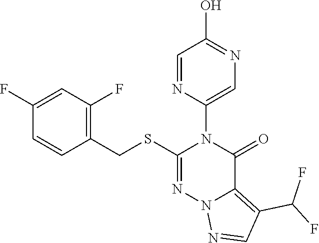 [SMILES: O=c1c2c(C(F)F)cnn2nc(SCc2ccc(F)cc2F)n1-c1cnc(O)cn1] | 439 [M + H]⁺ | 0.81 (X018_S03) |
| 114 | 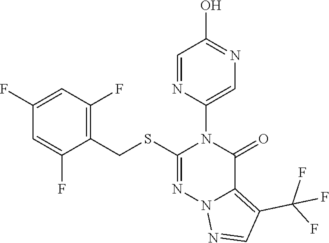 [SMILES: O=c1c2c(C(F)(F)F)cnn2nc(SCc2c(F)cc(F)cc2F)n1-c1cnc(O)cn1] | 473 [M − H]⁻ | 0.96 (Z018_S04) |
| 115 | 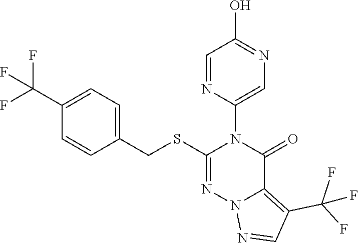 [SMILES: O=c1c2c(C(F)(F)F)cnn2nc(SCc2ccc(C(F)(F)F)cc2)n1-c1cnc(O)cn1] | 487 [M − H]⁻ | 1.00 (Z018_S04) |
| 116 | 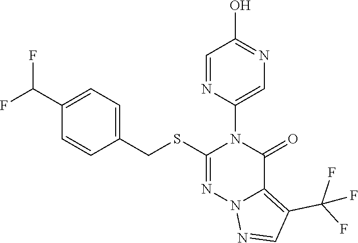 [SMILES: O=c1c2c(C(F)(F)F)cnn2nc(SCc2ccc(C(F)F)cc2)n1-c1cnc(O)cn1] | 469 [M − H]⁻ | 0.94 (Z018_S04) |
| 117 | 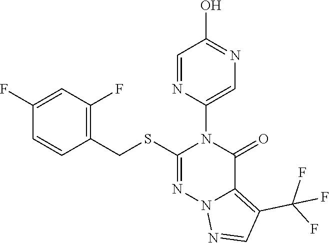 [SMILES: O=c1c2c(C(F)(F)F)cnn2nc(SCc2ccc(F)cc2F)n1-c1cnc(O)cn1] | 455 [M − H]⁻ | 0.94 (Z018_S04) |
| 118 | 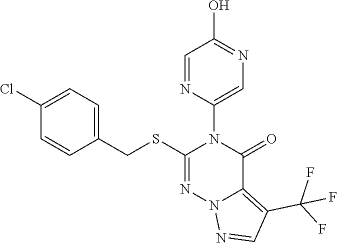 [SMILES: O=c1c2c(C(F)(F)F)cnn2nc(SCc2ccc(Cl)cc2)n1-c1cnc(O)cn1] | 453 [M − H]⁻ | 0.97 (Z018_S04) |
| 119 | 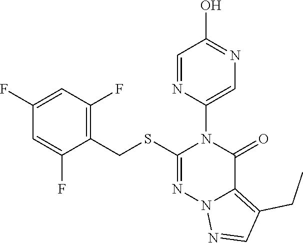 [SMILES: CCc1cnn2nc(SCc3c(F)cc(F)cc3F)n(-c3cnc(O)cn3)c(=O)c12] | 435 [M + H]⁺ | 0.57 (X012_S01) |
| 120 | 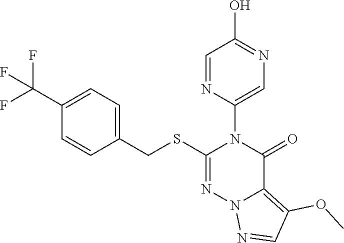 [SMILES: COc1cnn2nc(SCc3ccc(C(F)(F)F)cc3)n(-c3cnc(O)cn3)c(=O)c12] | 451 [M + H]⁺ | 0.92 (Z018_S04) |
| 121 | 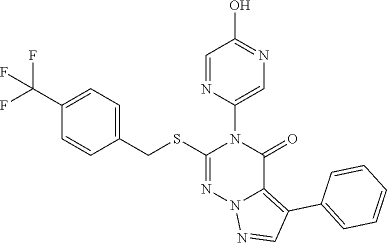 [SMILES: O=c1c2c(-c3ccccc3)cnn2nc(SCc2ccc(C(F)(F)F)cc2)n1-c1cnc(O)cn1] | 497 [M + H]⁺ | 1.04 (Z018_S04) |
| 122 | 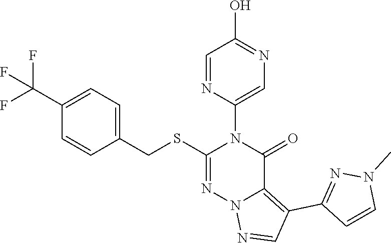 [SMILES: Cn1ccc(-c2cnn3nc(SCc4ccc(C(F)(F)F)cc4)n(-c4cnc(O)cn4)c(=O)c23)n1] | 501 [M + H]⁺ | 0.92 (Z018_S04) |
| 123 | 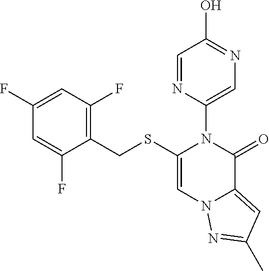 [SMILES: Cc1cc2c(=O)n(-c3cnc(O)cn3)c(SCc3c(F)cc(F)cc3F)cn2n1] | 421 [M + H]⁺ | 0.51 (X012_S01) |
| 124 | 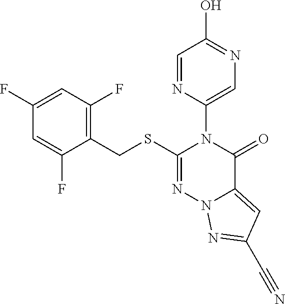 [SMILES: N#Cc1cc2c(=O)n(-c3cnc(O)cn3)c(SCc3c(F)cc(F)cc3F)nn2n1] | 432 [M + H]⁺ | 0.54 (X012_S01) |
| 125 | 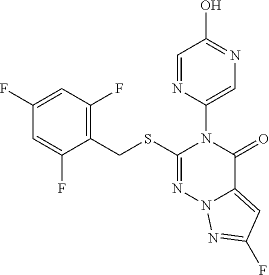 [SMILES: O=c1c2cc(F)nn2nc(SCc2c(F)cc(F)cc2F)n1-c1cnc(O)cn1] | 425 [M + H]⁺ | 0.78 (X018_S03) |
| 126 | 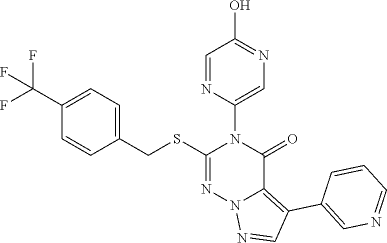 [SMILES: O=c1c2c(-c3cccnc3)cnn2nc(SCc2ccc(C(F)(F)F)cc2)n1-c1cnc(O)cn1] | 498 [M + H]⁺ | 0.77 (Z018_S04) |

### Example 127

### 3-(5-Fluoro-6-hydroxypyridin-3-yl)-2-((2,4,6-trifluorobenzyl)thio)pyrazolo[5,1-f][1,2,4]-triazin-4(3H)-one

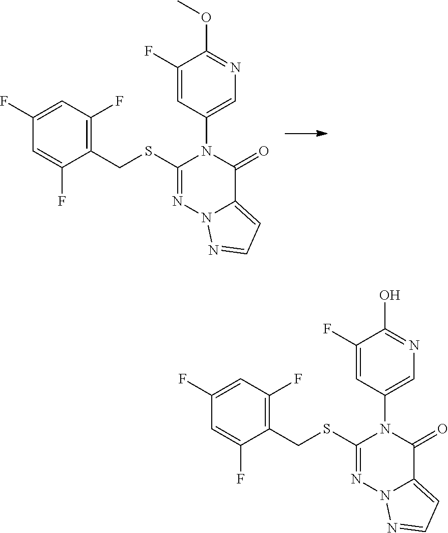 [SMILES: COc1ncc(-n2c(SCc3c(F)cc(F)cc3F)nn3nccc3c2=O)cc1F ; O=c1c2ccnn2nc(SCc2c(F)cc(F)cc2F)n1-c1cnc(O)c(F)c1]

Trimethylsilyl chloride (235 μL, 1.85 μmol, 4.50 equiv) is added to mixture of 3-(5-fluoro-6-methoxypyridin-3-yl)-2-((2,4,6-trifluorobenzyl)thio)pyrazolo[5,1-f][1,2,4]triazin-4(3H)-one (intermediate 62, 180 mg, 412 μmol, 1 equiv) and potassium iodide (61.5 mg, 370 μmol, 0.900 equiv) in acetonitrile (3 mL). After 18 h, water is added, and the resulting precipitate is filtered and dried to yield the desired product.

| | |
|---|---|
| C₁₇H₉F₄N₅O₂S | (MW = 423.0 g/mol) |
| ESI | 424 [M + H]⁺ |
| Rₜ (HPLC) | 0.84 min (Z018_S04) |

The following examples are prepared using procedures analogous to those described for example 127, using appropriate starting materials. As is appreciated by those skilled in the art, the analogous examples may involve variations in general reaction conditions.

| | | | HPLC retention |
|---|---|---|---|
| | | | time |
| Example | Structure | ESI-MS | [min] (method) |
| 128 | 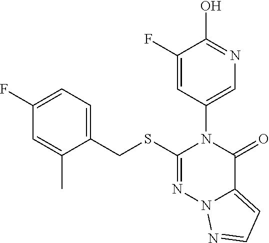 [SMILES: Cc1cc(F)ccc1CSc1nn2nccc2c(=O)n1-c1cnc(O)c(F)c1] | 402 [M + H]⁺ | 0.87 (Z018_S04) |
| 129 | 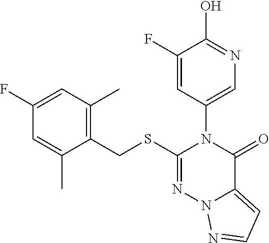 [SMILES: Cc1cc(F)cc(C)c1CSc1nn2nccc2c(=O)n1-c1cnc(O)c(F)c1] | 416 [M + H]⁺ | 0.91 (Z018_S04) |
| 130 | 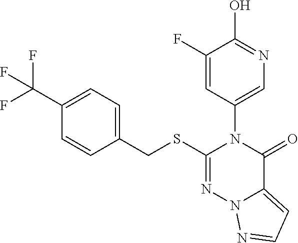 [SMILES: O=c1c2ccnn2nc(SCc2ccc(C(F)(F)F)cc2)n1-c1cnc(O)c(F)c1] | 438 [M + H]⁺ | 0.90 (Z018_S04) |
| 131 | 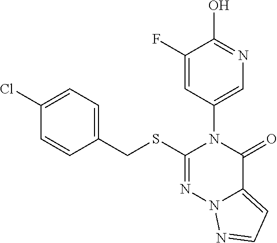 [SMILES: O=c1c2ccnn2nc(SCc2ccc(Cl)cc2)n1-c1cnc(O)c(F)c1] | 404/406 [M + H]⁺ | 0.87 (Z018_S04) |
| 132 | 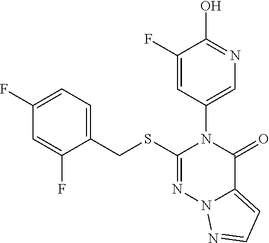 [SMILES: O=c1c2ccnn2nc(SCc2ccc(F)cc2F)n1-c1cnc(O)c(F)c1] | 406 [M + H]⁺ | 0.83 (Z018_S04) |
| 133 | 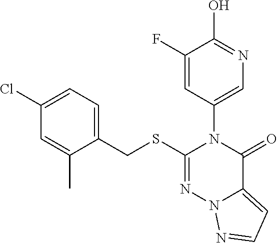 [SMILES: Cc1cc(Cl)ccc1CSc1nn2nccc2c(=O)n1-c1cnc(O)c(F)c1] | 418/420 [M + H]⁺ | 0.92 (Z018_S04) |
| 134 | 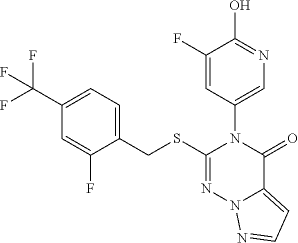 [SMILES: O=c1c2ccnn2nc(SCc2ccc(C(F)(F)F)cc2F)n1-c1cnc(O)c(F)c1] | 456 [M + H]⁺ | 0.92 (Z018_S04) |
| 135 | 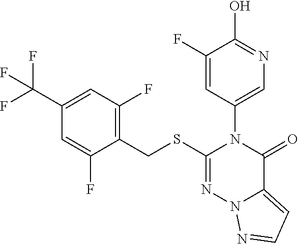 [SMILES: O=c1c2ccnn2nc(SCc2c(F)cc(C(F)(F)F)cc2F)n1-c1cnc(O)c(F)c1] | 472 [M − H]⁻ | 0.92 (Z018_S04) |
| 136 | 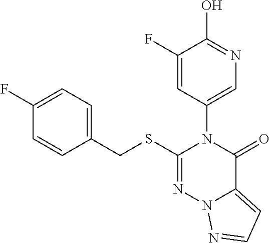 [SMILES: O=c1c2ccnn2nc(SCc2ccc(F)cc2)n1-c1cnc(O)c(F)c1] | 386 [M − H]⁻ | 0.83 (Z018_S04) |
| 137 | 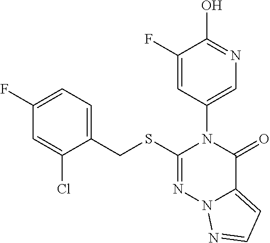 [SMILES: O=c1c2ccnn2nc(SCc2ccc(F)cc2Cl)n1-c1cnc(O)c(F)c1] | 422 [M + H]⁺ | 0.88 (Z018_S04) |
| 138 | 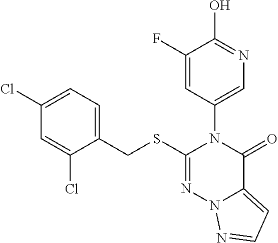 [SMILES: O=c1c2ccnn2nc(SCc2ccc(Cl)cc2Cl)n1-c1cnc(O)c(F)c1] | 438 [M + H]⁺ | 0.93 (Z018_S04) |
| 139 | 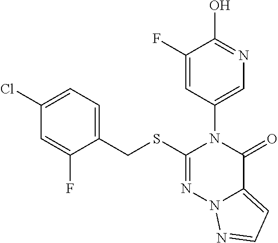 [SMILES: O=c1c2ccnn2nc(SCc2ccc(Cl)cc2F)n1-c1cnc(O)c(F)c1] | 420 [M − H]⁻ | 0.90 (Z018_S04) |
| 140 | 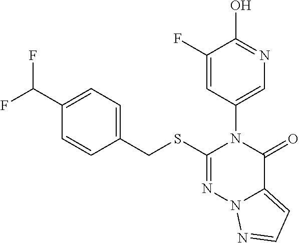 [SMILES: O=c1c2ccnn2nc(SCc2ccc(C(F)F)cc2)n1-c1cnc(O)c(F)c1] | 420 [M + H]⁺ | 0.85 (Z018_S04) |
| 141 | 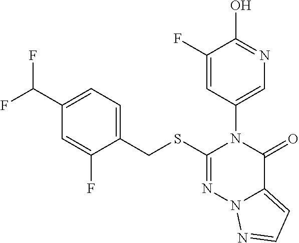 [SMILES: O=c1c2ccnn2nc(SCc2ccc(C(F)F)cc2F)n1-c1cnc(O)c(F)c1] | 436 [M − H]⁻ | 0.86 (Z018_S04) |
| 142 | 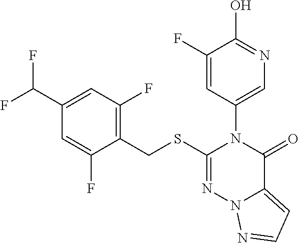 [SMILES: O=c1c2ccnn2nc(SCc2c(F)cc(C(F)F)cc2F)n1-c1cnc(O)c(F)c1] | 456 [M + H]⁺ | 0.87 (Z018_S04) |
| 143 | 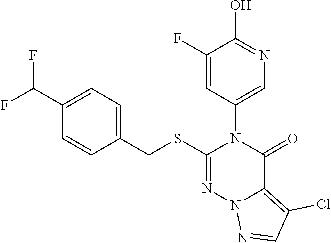 [SMILES: O=c1c2c(Cl)cnn2nc(SCc2ccc(C(F)F)cc2)n1-c1cnc(O)c(F)c1] | 452 [M − H]⁻ | 0.91 (Z018_S04) |
| 144 | 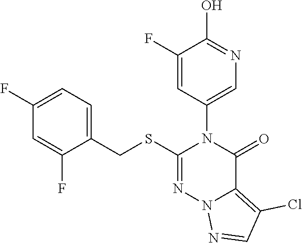 [SMILES: O=c1c2c(Cl)cnn2nc(SCc2ccc(F)cc2F)n1-c1cnc(O)c(F)c1] | 438 [M − H]⁻ | 0.91 (Z018_S04) |
| 145 | 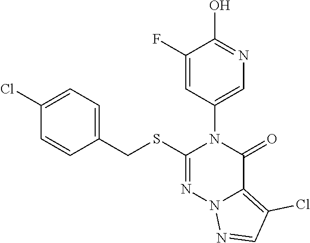 [SMILES: O=c1c2c(Cl)cnn2nc(SCc2ccc(Cl)cc2)n1-c1cnc(O)c(F)c1] | 436 [M − H]⁻ | 0.94 (Z018_S04) |
| 146 | 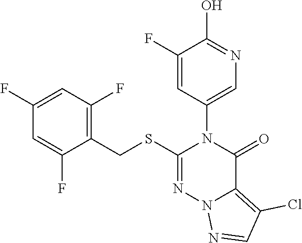 [SMILES: O=c1c2c(Cl)cnn2nc(SCc2c(F)cc(F)cc2F)n1-c1cnc(O)c(F)c1] | 456 [M − H]⁻ | 0.90 (Z018_S04) |
| 147 | 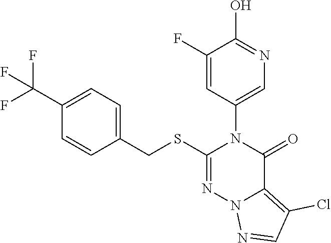 [SMILES: O=c1c2c(Cl)cnn2nc(SCc2ccc(C(F)(F)F)cc2)n1-c1cnc(O)c(F)c1] | 470 [M − H]⁻ | 0.97 (Z018_S04) |
| 148 | 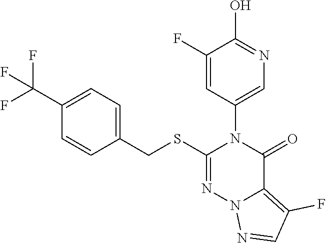 [SMILES: O=c1c2c(F)cnn2nc(SCc2ccc(C(F)(F)F)cc2)n1-c1cnc(O)c(F)c1] | 456 [M + H]⁺ | 0.93 (Z018_S04) |
| 149 | 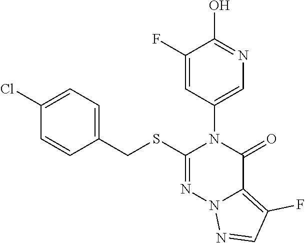 [SMILES: O=c1c2c(F)cnn2nc(SCc2ccc(Cl)cc2)n1-c1cnc(O)c(F)c1] | 422/424 [M + H]⁺ | 0.91 (Z018_S04) |
| 150 | 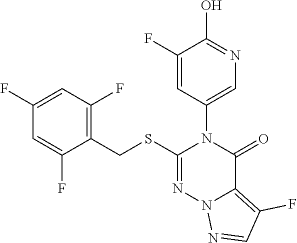 [SMILES: O=c1c2c(F)cnn2nc(SCc2c(F)cc(F)cc2F)n1-c1cnc(O)c(F)c1] | 442 [M + H]⁺ | 0.88 (Z018_S04) |
| 151 | 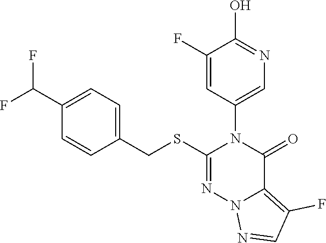 [SMILES: O=c1c2c(F)cnn2nc(SCc2ccc(C(F)F)cc2)n1-c1cnc(O)c(F)c1] | 438 [M + H]⁺ | 0.88 (Z018_S04) |
| 152 | 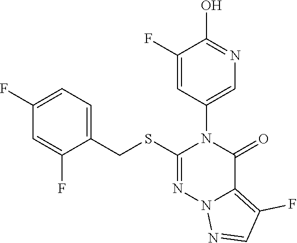 [SMILES: O=c1c2c(F)cnn2nc(SCc2ccc(F)cc2F)n1-c1cnc(O)c(F)c1] | 424 [M + H]⁺ | 0.87 (Z018_S04) |
| 153 | 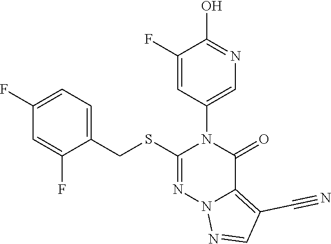 [SMILES: N#Cc1cnn2nc(SCc3ccc(F)cc3F)n(-c3cnc(O)c(F)c3)c(=O)c12] | 431 [M + H]⁺ | 0.87 (Z018_S04) |
| 154 | 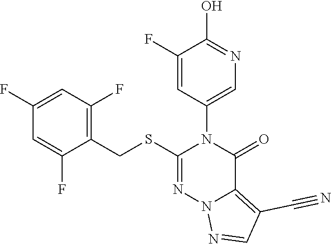 [SMILES: N#Cc1cnn2nc(SCc3c(F)cc(F)cc3F)n(-c3cnc(O)c(F)c3)c(=O)c12] | 449 [M + H]⁺ | 0.88 (Z018_S04) |
| 155 | 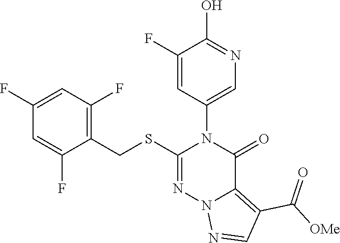 [SMILES: COC(=O)c1cnn2nc(SCc3c(F)cc(F)cc3F)n(-c3cnc(O)c(F)c3)c(=O)c12] | 482 [M + H]⁺ | 0.87 (Z018_S04) |
| 156 | 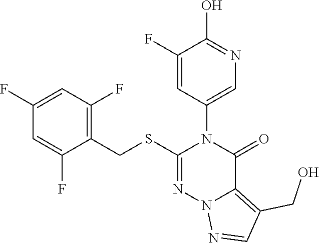 [SMILES: O=c1c2c(CO)cnn2nc(SCc2c(F)cc(F)cc2F)n1-c1cnc(O)c(F)c1] | 452 [M − H]⁻ | 0.80 (Z018_S04) |
| 157 | 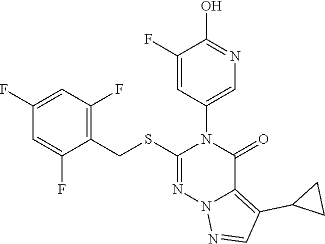 [SMILES: O=c1c2c(C3CC3)cnn2nc(SCc2c(F)cc(F)cc2F)n1-c1cnc(O)c(F)c1] | 462 [M − H]⁻ | 0.59 (X012_S01) |

### Example 158

### 2-((4-Chlorobenzyl)thio)-3-(5-fluoro-6-hydroxypyridin-3-yl)-5-methylpyrazolo[5,1-f][1,2,4]triazin-4(3H)-one

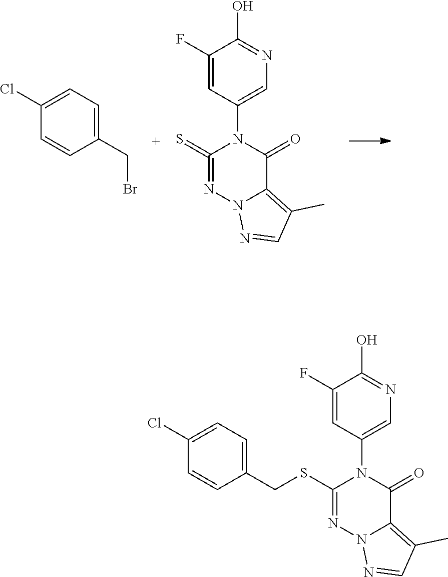

1-(Bromomethyl)-4-chlorobenzene (32.0 mg, 151 μmol, 0.700 equiv) is added to a mixture of triethylamine (57.0 μL, 409 μmol, 2.00 equiv) and 3-(5-fluoro-6-hydroxypyridin-3-yl)-5-methyl-2-thioxo-2,3-dihydropyrazolo[5,1-f][1,2,4]triazin-4(1H)-one (intermediate 93, 60.0 mg, 205 μmol, 1 equiv) in methanol (1 mL). After 1.5 h, the reaction mixture is filtered and the filtrate is purified by reversed phase HPLC (XBridge™-C18, gradient of acetonitrile in water, 0.1% TFA) to yield the desired products.

| | |
|---|---|
| C₁₈H₁₃ClFN₅O₂S | (MW = 417.0 g/mol) |
| ESI | 416 [M − H]⁻ |
| Rₜ (HPLC) | 0.57 min (X012_S01) |

The following intermediates are prepared using procedures analogous to those described for example 158, using appropriate starting materials. As is appreciated by those skilled in the art, the analogous examples may involve variations in general reaction conditions.

| | | | HPLC retention |
|---|---|---|---|
| Example | Structure | ESI-MS | time [min] (method) |
| 159 | 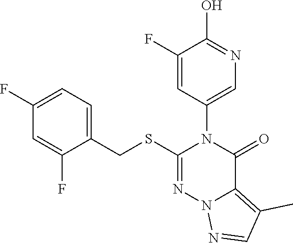 [SMILES: Cc1cnn2nc(SCc3ccc(F)cc3F)n(-c3cnc(O)c(F)c3)c(=O)c12] | 418 [M − H]⁻ | 0.53 (X012_S01) |
| 160 | 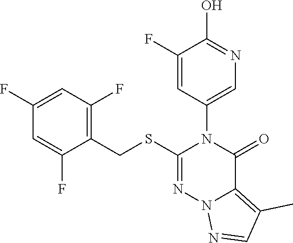 [SMILES: Cc1cnn2nc(SCc3c(F)cc(F)cc3F)n(-c3cnc(O)c(F)c3)c(=O)c12] | 438 [M + H]⁺ | 0.53 (X012_S01) |
| 161 | 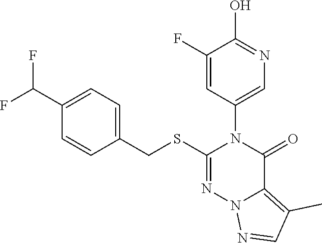 [SMILES: Cc1cnn2nc(SCc3ccc(C(F)F)cc3)n(-c3cnc(O)c(F)c3)c(=O)c12] | 434 [M + H]⁺ | 0.54 (X012_S01) |
| 162 | 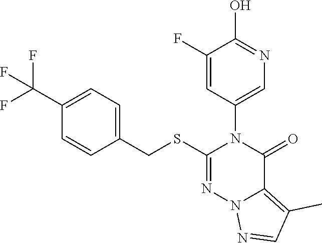 [SMILES: Cc1cnn2nc(SCc3ccc(C(F)(F)F)cc3)n(-c3cnc(O)c(F)c3)c(=O)c12] | 452 [M + H]⁺ | 0.60 (X012_S01) |
| 163 | 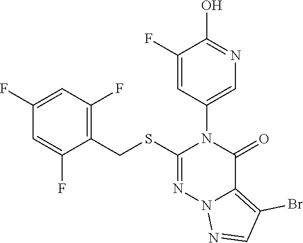 [SMILES: O=c1c2c(Br)cnn2nc(SCc2c(F)cc(F)cc2F)n1-c1cnc(O)c(F)c1] | 500 [M − H]⁻ | 0.57 (X012_S01) |

### Example 164

### 2-((4-Chlorobenzyl) thio)-5-fluoro-3-(6-hydroxypyridin-3-yl)pyrazolo[5,1-f][1,2,4]triazin-4(3H)-one

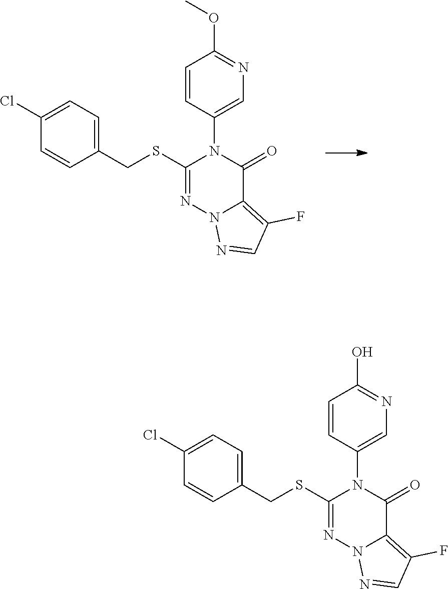 [SMILES: COc1ccc(-n2c(SCc3ccc(Cl)cc3)nn3ncc(F)c3c2=O)cn1 ; O=c1c2c(F)cnn2nc(SCc2ccc(Cl)cc2)n1-c1ccc(O)nc1]

Example 164 is prepared using procedures analogous to those described for example 127, using appropriate starting materials.

| | |
|---|---|
| C₁₇H₁₁ClFN₅O₂S | (MW = 403.0 g/mol) |
| ESI | 404/406 [M + H]⁺ |
| Rₜ (HPLC) | 0.90 min (Z018_S04) |

The following examples are prepared using procedures analogous to those described for example 164, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 165 | 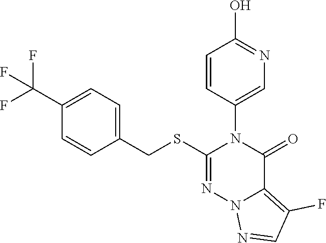 [SMILES: O=c1c2c(F)cnn2nc(SCc2ccc(C(F)(F)F)cc2)n1-c1ccc(O)nc1] | 438 [M + H]⁺ | 0.92 (Z018_S04) |
| 166 | 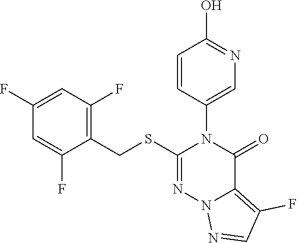 [SMILES: O=c1c2c(F)cnn2nc(SCc2c(F)cc(F)cc2F)n1-c1ccc(O)nc1] | 424 [M + H]⁺ | 0.86 (Z018_S04) |
| 167 | 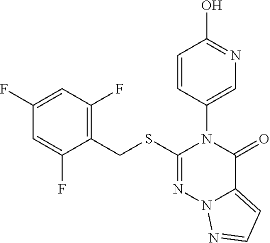 [SMILES: O=c1c2ccnn2nc(SCc2c(F)cc(F)cc2F)n1-c1ccc(O)nc1] | 406 [M + H]⁺ | 0.81 (Z018_S04) |

### Analytical HPLC Methods

| | |
|---|---|
| Method Name: | X011_S05 |
| Device description: | Waters Acquity with DA- and MS-Detector |
| Column: | XBridge BEH C18_2.1 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% NH3] | [Acetonitrile] | [ml/min] | [° C.] |
| 0.0 | 95.0 | 5.0 | 1.3 | 60.0 |
| 0.02 | 95.0 | 5.0 | 1.3 | 60.0 |
| 1.0 | 0.0 | 100.0 | 1.3 | 60.0 |
| 1.3 | 0.0 | 100.0 | 1.3 | 60.0 |

| | |
|---|---|
| Method Name: | X012_S01 |
| Device description: | Waters Acquity with DA- and MS-Detector |
| Column: | XBridge BEH C18_2.1 × 30 mm_1.7 μm |
| Column producer: | Waters |
| Description: | |

| Gradient/Solvent | % Sol [Water | % Sol | Flow | Temp |
|---|---|---|---|---|
| Time [min] | 0.1% TFA (v/v)] | [Acetonitrile] | [ml/min] | [° C.] |
| 0.0 | 99.0 | 1.0 | 1.6 | 60.0 |
| 0.02 | 99.0 | 1.0 | 1.6 | 60.0 |
| 1.0 | 0.0 | 100.0 | 1.6 | 60.0 |
| 1.1 | 0.0 | 100.0 | 1.6 | 60.0 |

| | |
|---|---|
| Method Name: | X018_S01 |
| Device description: | Waters Acquity with DA- and MS-Detector |
| Column: | Sunfire C18_2.1 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% TFA (v/v)] | [Acetonitrile] | [ml/min] | [° C.] |
| 0.0 | 99.0 | 1.0 | 1.5 | 60.0 |
| 0.02 | 99.0 | 1.0 | 1.5 | 60.0 |
| 1.0 | 0.0 | 100.0 | 1.5 | 60.0 |
| 1.1 | 0.0 | 100.0 | 1.5 | 60.0 |

| | |
|---|---|
| Method Name: | X018_S03 |
| Device description: | Waters Acquity with DA- and MS-Detector |
| Column: | Sunfire C18_3.0 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% TFA (v/v)] | [Acetonitrile] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |

| | |
|---|---|
| Method Name: | Z011_S03 |
| Device description: | Agilent 1200 with DA- and MS-Detector |
| Column: | XBridge C18_3.0 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% NH3] | [Acetonitrile] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | Z018_S04 |
| Device description: | Agilent 1200 with DA- and MS-Detector |
| Column: | Sunfire C18_3.0 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% TFA (v/v)] | [Acetonitrile] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | X011_U04 |
| Device description: | Waters Acquity with DA- and MS-Detector |
| Column: | XBridge BEH C18_2.1 × 30 mm_2.5 μm |
| Column producer: | Waters |
| Description: | |

| Gradient/Solvent | % Sol [Water | % Sol | Flow | Temp |
|---|---|---|---|---|
| Time [min] | 0.1% NH3 (v/v)] | [Acetonitrile] | [ml/min] | [° C.] |
| 0.0 | 50.0 | 50.0 | 1.3 | 60.0 |
| 0.02 | 50.0 | 50.0 | 1.3 | 60.0 |
| 1.0 | 0.0 | 100.0 | 1.3 | 60.0 |
| 1.2 | 0.0 | 100.0 | 1.3 | 60.0 |

1. A compound according to general formula (Ia) 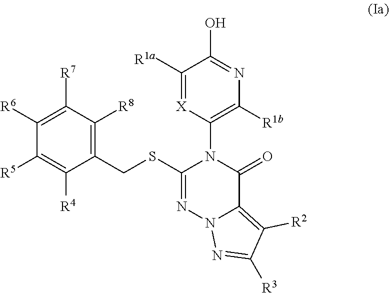
wherein
X is N or C—R^{1c};
R^{1c} is H;
R^{1a}, and R^{1b} are, independently of each other, H or F;
R² is selected from the group consisting of —H, -halogen, —C₁₋₄-alkyl, -cyclopropyl, —C₁₋₄-haloalkyl, —CN, —C₂₋₄-alken-1-yl, —C₂₋₄-haloalken-1-yl, —C₂₋₄-alkyn-1-yl, —O—C₁₋₄-alkyl, —CO₂C₁₋₄-alkyl, —C(═O)NH₂, —C(═O)NH—C₁₋₄-alkyl, —NH₂, —NO₂, —C₁₋₄-alkyl-OH, —C₁₋₄-alkyl-O—C₁₋₄-alkyl, —C(═O)—C₁₋₄-alkyl, —C(═O)H, —CH═NOH, —CH═NOCH₃, phenyl and 5-6-membered heteroaryl optionally substituted with —C₁₋₄-alkyl;
R³ is selected from the group consisting of —H, —C₁₋₄-alkyl, —CN, and -halogen;
R⁴, R⁵, R⁶, R⁷ and R⁸ are, independently of each other, selected from the group consisting of —H, -halogen, —C₁₋₄-alkyl, -cyclopropyl, —C₂₋₄-alken-1-yl, —C₂₋₄-haloalken-1-yl, —C₂₋₄-alkyn-1-yl, —C₁₋₄-haloalkyl, —OCH₃, —CN and —SF₅;
or a salt thereof.

2. The compound according to claim 1, wherein X is N, or a salt thereof.

3. The compound according to claim 1, wherein X is C—R^{1c}, or a salt thereof.

4. The compound according to claim 1, wherein
R⁴, R⁵, R⁶, R⁷, and R⁸ are, independently of each other, selected from among a group consisting of H, —F, Cl, —C₁₋₂-alkyl, -cyclopropyl, —CH═CH₂, —CH═CHF, —CH═CF₂, —C≡CH, —C≡CCH₃, —CF₃, —CF₂H, —CF₂Cl, —OCH₃, —CN and —SF₅; or a salt thereof.

5. The compound according to claim 1, wherein
R² is selected from the group consisting of —H, -halogen, —C₁₋₄-alkyl and —C₁₋₄-haloalkyl;
and R³ is H; or a salt thereof.

6. The compound according to claim 1, wherein
R² is selected from among a group consisting of -cyclopropyl, —CN, —C₂₋₄-alkyn-1-yl, —C₂₋₄-alken-1-yl, —C₂₋₄-haloalken-1-yl, —O—C₁₋₄-alkyl, —CO₂C₁₋₄-alkyl, —C(═O)NH₂, —C(═O)NHC₁₋₄-alkyl, —NH₂, —NO₂, —C₁₋₄-alkyl-OH, —C₁₋₄-alkyl-O—C₁₋₄-alkyl, —C(═O)C₁₋₄-alkyl, —C(═O)H, —CH═NOH, —CH═NOCH₃, phenyl, 2-pyridyl, 3-pyridyl, and 1-methyl-pyrazol-3-yl; and
R³ is H; or a salt thereof.

7. The compound according to claim 1, wherein
R² is H; and
R³ is selected from the group consisting of —F, —CH₃ and —CN; or a salt thereof.

8. The compound according to claim 1, selected from the group consisting of 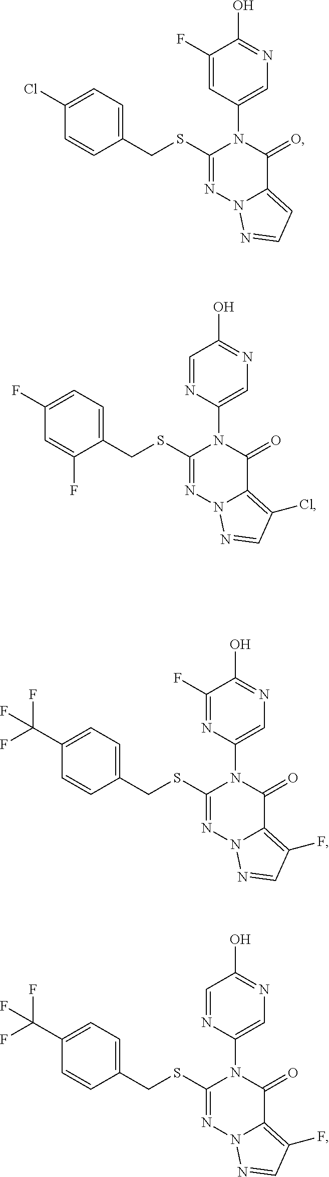 [SMILES: O=c1c2ccnn2nc(SCc2ccc(Cl)cc2)n1-c1cnc(O)c(F)c1 ; O=c1c2c(Cl)cnn2nc(SCc2ccc(F)cc2F)n1-c1cnc(O)cn1 ; O=c1c2c(F)cnn2nc(SCc2ccc(C(F)(F)F)cc2)n1-c1cnc(O)c(F)n1 ; O=c1c2c(F)cnn2nc(SCc2ccc(C(F)(F)F)cc2)n1-c1cnc(O)cn1] 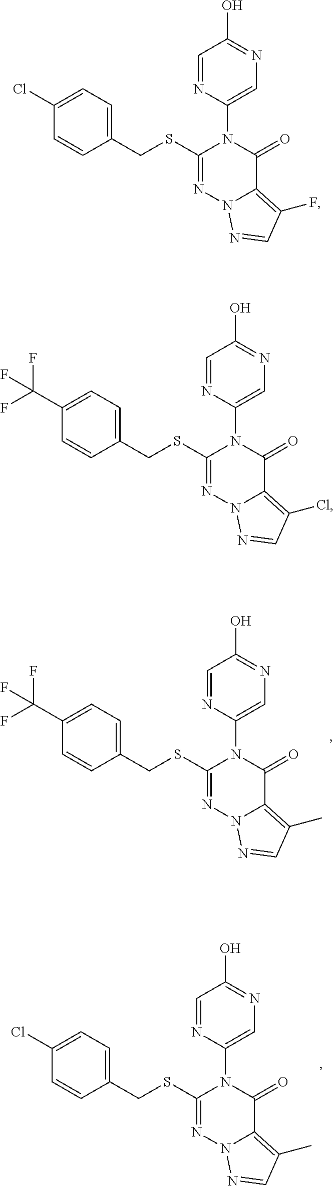 [SMILES: O=c1c2c(F)cnn2nc(SCc2ccc(Cl)cc2)n1-c1cnc(O)cn1 ; O=c1c2c(Cl)cnn2nc(SCc2ccc(C(F)(F)F)cc2)n1-c1cnc(O)cn1 ; Cc1cnn2nc(SCc3ccc(C(F)(F)F)cc3)n(-c3cnc(O)cn3)c(=O)c12 ; Cc1cnn2nc(SCc3ccc(Cl)cc3)n(-c3cnc(O)cn3)c(=O)c12] 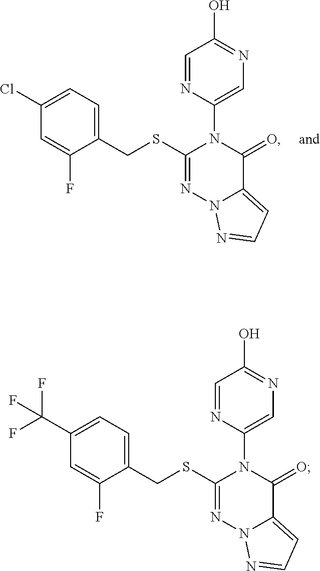 [SMILES: O=c1c2ccnn2nc(SCc2ccc(Cl)cc2F)n1-c1cnc(O)cn1 ; O=c1c2ccnn2nc(SCc2ccc(C(F)(F)F)cc2F)n1-c1cnc(O)cn1]
or a salt thereof.

9. The compound according to claim 8, wherein the compound is in the form of a pharmaceutically acceptable salt.

10. A pharmaceutical composition comprising at least one compound according to claim 1, or a pharmaceutically acceptable salt thereof, and a pharmaceutically acceptable carrier.

11. A pharmaceutical composition according to claim 10, further comprising an additional therapeutic agent selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRAS-G12D-inhibitors, MDM2-p53-antagonists, of Her2-inhibitors and chemotherapeutics.

12. A method for the treatment and/or prevention of a disease selected from the group consisting of chronic liver diseases, portal hypertension, viral infections, cancer, interstitial lung diseases, retinopathies, acute and chronic inflammation, and fibrotic diseases, said method comprising administering an effective amount of a compound according to claim 1, or a pharmaceutically acceptable salt thereof, to a human being.

13. The method according to claim 12 wherein the disease is selected from the group consisting of vascular inflammation, atherosclerosis, interstitial lung diseases, idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors, NOX4+ cancer associated fibroblast rich tumors wherein the tumor is selected in the group of pancreatic, lung, breast, colon, head and neck tumors, systemic sclerosis, inflammatory bowel disease, Duchenne muscular dystrophy, COVID-19, acute respiratory distress syndrome, influenza, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.
